(19) United States
(12) Patent Application Publication
(10) Pub. No.: US 2026/0347489 A1
(43) Pub. Date: Nov. 5, 2026

(54) ANTI-TL1A ANTIBODY THERAPEUTIC METHODS

(71) Applicants: Genentech, Inc., South San Francisco, CA (US); Pfizer Inc., New York, NY (US)

(72) Inventors: Sudhir PENUGONDA, Chicago, IL (US); Srini RAMANATHAN, San Carlos, CA (US); Jason CHAMBERLAIN, Reno, NV (US); Keith HAAN, Princeton, NJ (US); Mayukh SUKHATME, Mamaroneck, NY (US); Frank TORTI, Lyme, NH (US); Christopher Ricardo BANFIELD, Woburn, MA (US); Deepa E. CHANDRA, Cambridge, MA (US); Mina HASSAN-ZAHRAEE, Cambridge, MA (US); Xinli HU, Sudbury, MA (US); Kenneth HUNG, Watertown, MA (US); Craig L. HYDE, Old Lyme, CT (US); Steven W. MARTIN, Lexington, MA (US); Srividya NEELAKANTAN, East Walpole, MA (US); Elena PEEVA, Tarrytown, NY (US); Michael S. VINCENT, Newton, MA (US); Ying WU, Lexington, MA (US); Zhan YE, Lexington, MA (US)

(21) Appl. No.: 19/734,181

(22) Filed: Jul. 8, 2026

Related U.S. Application Data
(63) Continuation of application No. 19/390,127, filed on Nov. 14, 2025, now abandoned.
Application No. 19/390,127 is a continuation of application No. PCT/US2024/030118, filed on May 17, 2024.
(60) Provisional application No. 63/502,786, filed on May 17, 2023.
(60) Provisional application No. 63/647,481, filed on May 14, 2024.
(60) Provisional application No. 63/649,269, filed on May 17, 2024.

(51) Int. Cl. C07K 16/28; A61K 9/00; A61K 39/00; A61K 45/06; A61P 1/00; C12Q 1/6874; C12Q 1/6883; C12Q 1/689

(52) U.S. Cl. CPC C07K 16/2875; A61K 9/0019; A61K 45/06; A61P 1/00; C12Q 1/6874; C12Q 1/6883; C12Q 1/689; A61K 2039/505; A61K 2039/54; A61K 2039/545; C07K 2317/565; C12Q 2600/106; C12Q 2600/156

(57) ABSTRACT

The present disclosure provides methods and compositions for determining the risk of a patient being non-responsive to a therapeutic dose of an anti-TNF-like ligand 1A (TL1A) antibody and methods and compositions for treating inflammatory bowel disease (IBD) with a therapeutic dose of an anti-TNF-like ligand 1A (TL1A) antibody.

### CROSS REFERENCE TO RELATED APPLICATIONS

This application is a continuation of U.S. patent application Ser. No. 19/390,127, filed Nov. 14, 2025, which is a continuation of International Patent Application No. PCT/US2024/030118, filed on May 17, 2024, which claims priority to U.S. Provisional Application No. 63/502,786, filed May 17, 2023; U.S. Provisional Application No. 63/647,481, filed May 14, 2024; and U.S. Provisional Application No. 63/649,269, filed May 17, 2024, the disclosures of which are herein incorporated by reference in their entirety.

### SEQUENCE LISTING

The instant application contains a Sequence Listing which has been submitted electronically in XML format and is hereby incorporated by reference in its entirety. Said XML copy, created on Jun. 30, 2026, is named 50474-352006_Sequence_Listing_6_30_26 and is 75,503 bytes in size.

### FIELD OF THE INVENTION

The present invention relates to the treatment of signs and symptoms of inflammatory bowel disease with an anti-tumor necrosis factor-like ligand 1A (TL1A) antibody.

### BACKGROUND OF THE INVENTION

Inflammatory bowel disease (IBD), which encompasses Crohn's disease and ulcerative colitis (UC), is a chronic inflammatory disorder affecting ~1.6 million people in the USA, and ~2.5-3 million people in Europe. To induce remission in patients with moderately to severely active UC, treatment recommendations include appropriate doses of oral corticosteroids, biologic therapies such as the tumor necrosis factor inhibitors (TNFi) infliximab (monotherapy or in combination with azathioprine), adalimumab, ustekinumab, golimumab, the integrin receptor antagonist vedolizumab, and the oral, small molecule Janus kinase inhibitor tofacitinib (Rubin et al, 2019 Am J Gastroenterol; 114:384-413). However, non-response and loss of response to treatments have been observed in UC and, therefore, the development of novel treatments for UC and IBD is still an unmet clinical need (Lichtenstein et al 2018 Am J Gastroenterol; 113:481-517).

A range of mucosal immune system components—including epithelial cells, innate and adaptive immune cells, cytokines and chemokines—contribute to the pathogenesis of IBD (Wallace et al, 2014, World J Gastroenterol; 20:6-21). One of the immune components involved in the pathogenesis of IBD is TNF-like ligand 1A (TL1A, or tissue necrosis factor superfamily member 15 (TNFSF15)). Genome-wide association studies have linked TNFSF15 single nucleotide polymorphisms with disease severity; for example, an association was observed between the rs11554257 single nucleotide polymorphism and medically refractory UC compared with healthy controls (Haritunians et al, 2010, Inflammatory bowel diseases; 16: 1830-1840). TL1A has been found to be upregulated in IBD tissue specimens, with level of expression corresponding to the severity of disease (Bamias et al, 2010, Clin Immunol; 137:242-249).

T cell-mediated signaling and cytokine production, for example, T helper (Th)1 cells producing interferon-γ, Th17 cells producing interleukin (IL)-6 and IL-17, and Th2 cells producing IL-4 and IL-13 are costimulated by TL1A binding to death receptor 3, (Migone et al, 2002, Immunity 16; 16:479-492; Takedatsu et al, 2008, Gastroenterology 135; 552-567; Meylan et al 2011; Mucosal Immunity 4; 172-185; Meylan et al 2008; Immunity 29; 79-89). Because increased cytokine production leads to chronic inflammation, inhibition of TL1A may be a therapeutic target for inflammatory diseases, including IBD.

UC is a chronic inflammatory disease of the large intestine characterized by diffuse mucosal inflammation. The underlying pathophysiology of this disease results from the interplay of genetic susceptibility in immune genes and alterations in the gut microbiome. Current treatments for UC target immune cell activation including non-selective medicines such as corticosteroids, mesalamine, thiopurines as well as selective biologic (anti-TNFa, anti-a4b7, and anti-IL-12/23 agents) and small molecule Janus kinase inhibitors. Despite these treatments, many patients have an inadequate response as determined by endoscopy and develop refractory disease. There is, therefore, an urgent need to develop therapeutics driving gastrointestinal healing determined by, e.g., endoscopy (referred to as “endoscopic healing”) and to define tissue, blood, and microbiome biomarkers to help guide therapy.

There is still an unmet need for an effective, safe, and well tolerated treatment in subjects with moderate to severe ulcerative colitis. The hallmark clinical symptoms of UC include bloody diarrhoea associated with rectal urgency and tenesmus. The clinical course is marked by exacerbation and remission. The diagnosis of UC is suspected on clinical grounds and supported by diagnostic testing, and elimination of infectious causes (Dignass et al., J Crohn's Colitis. 2012; 6(10):965-90). The most severe intestinal manifestations of UC are toxic megacolon and perforation. Extraintestinal complications include arthritis (peripheral or axial involvement), dermatological conditions (erythema nodosum, aphthous stomatitis, and pyoderma gangrenosum), inflammation of the eye (uveitis), and liver dysfunction (primary sclerosing cholangitis). Subjects with UC are at an increased risk for colon cancer, and the risk increases with the duration of disease as well as extent of colon affected by the disease (Rutter et al, 2004 Gastroenterology; 126(2):451-9).

The aim of medical treatment in UC is to control inflammation and reduce symptoms. Available pharmaceutical therapies are limited, do not always completely abate the inflammatory process and may have significant adverse effects. Therapies for mild to moderate active UC include 5-aminosalicylic acid derivatives and immunosuppressants.

Genome-wide association studies have linked over 200 genes with IBD, with many of these genes associated with this underlying dysregulated immunoregulatory functions (de Lange et al 2017, Nat Genet.; 49(2): 256-610). One of the strongest genetic variants associated with IBD exist in the Tumor Necrosis Factor Superfamily member 15 (TNFSF15) locus (Siakavellas et al 2015; Inflamm. Bowel Dis. 21 (10): 2441-52). Variants in TNFSF15 have been linked to the pathogenesis of several autoimmune diseases—including psoriasis, rheumatoid arthritis, and multiple sclerosis—implicating a broad role for TNFSF15 in human inflammatory diseases. In IBD, TNFSF15 variants may confer higher risk for more aggressive, penetrating, fibrostenotic, and perianal disease complications (Yang et al 2014 J Crohns Colitis; 8(10): 1315-26; Tung et al 2014 J Gasteroenterl Hepatol; 29(4):273-9). TNFSF15 encodes for the protein TNF-like ligand 1A (TL1A), which is highly expressed in human colonic tissue during active colitis (Bamias et al 2013; Curr Opinion Gasteroenterol. 29(6): 597-602).

The mechanistic impact of TL1A in pre-clinical models is pleiotropic. While early studies have shown a pathogenic role for TL1A overexpression in driving inflammatory Th1, Th17, and group 2 innate lymphoid cell responses, more recent reports in mouse models of acute colitis and ileitis revealed a contrasting protective role for endogenous TL1A in supporting anti-inflammatory T regulatory cells (Treg) and group 3 innate lymphoid cell function (Prehn et al, 2004, Clin Immunol 112(1):66-77; Castellanos, et al 2019; Mucosal Immunol.11(5): 1466-1476). In addition to the impact on lymphoid cells, mouse models have revealed a key impact of TL1A overexpression in intestinal fibrosis. Moreover, in vitro studies of peripheral blood macrophage revealed a contribution of the TNFSF15 risk haplotype in synergistically regulating NOD2 ligand induced inflammatory cytokines (Hedl and Abraham, 2014; PNAS 111 (37) 13451-13456. Collectively, these pre-clinical studies highlight a potential central homeostatic role for TL1A in modulating selective innate and adaptive immune pathways critical for IBD, as well as the key clinical complication of fibrosis. To date, no studies have defined the mechanisms underlying the potential efficacy of anti-TL1A therapy in humans.

Pre-clinical studies in rodent colitis models and human cells have shown that anti-TL1A antibodies can reduce tissue fibrosis, the number of fibroblasts, and clinical disease score, thereby highlighting their potential as biologic therapies for IBD (Clarke et al 2002, MAbs 10:664-677; Shih et al 2014 Mucosal Immunol; 7:1492-1503).

There is a need in the art for treating inflammatory bowel disease, including Crohn's disease and ulcerative colitis, with anti-TNF-like ligand 1A (TL1A) antibody therapy and a need for identifying patients who may be responsive or non-responsive to such therapies. The present disclosure addresses these needs.

### SUMMARY OF THE INVENTION

The present disclosure provides a method of determining the risk of a patient being non-responsive to a therapeutic dose of an anti-TNF-like ligand 1A (TL1A) antibody comprising performing a genotyping assay on a biological sample from the patient to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient, and determining the risk of being non-responsive to the therapeutic dose of an anti-TL1A antibody, wherein the risk is higher in a haplotype B carrier patient than in a haplotype B non-carrier patient. The patient can have inflammatory bowel disease (IBD).

The present disclosure provides a method for treating inflammatory bowel disease (IBD) in a patient, the method comprising (a) performing a genotyping assay on a biological sample from the patient to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and (b) administering to the patient an anti-TNF-like ligand 1A (TL1A) antibody, wherein the patient has been determined to be a haplotype B non-carrier.

In some aspects, the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses.

In some aspects, the anti-TL1A antibody is administered to the patient in a maintenance dosing regimen after completion of an induction dosing regimen, said maintenance dosing regimen comprising a plurality of individual maintenance doses separated from each other by at least 2 weeks.

In some aspects, the disclosure features a method for treating IBD in a patient, the method comprising (a) performing a genotyping assay to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; (b) administering to the patient an anti-TL1A antibody in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and (c) administering to the patient a subsequent maintenance dosing regimen after completion of the induction dosing regimen, said maintenance dosing regimen comprising a plurality of individual maintenance doses separated from each other by at least 2 weeks.

The IBD can be ulcerative colitis (UC). The UC can be moderate to severe ulcerative colitis. In some aspects, the IBD is Crohn's disease (CD).

In a preferred aspect, the at least two SNPs are SNP rs3810936 and SNP rs7869487.

In some aspects, the genotyping assay comprises determining the presence of only two SNPs in the biological sample from the patient, wherein the two SNPs are SNP rs3810936 and SNP rs7869487.

In some aspects, the patient is determined to be a haplotype B non-carrier by any one of the following biomarker statuses in the biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487.

In some aspects, (a) the genotyping assay comprises determining the presence of SNP rs3810936, SNP rs7869487, and SNP rs7848647 in the biological sample from the patient; or (b) the genotyping assay comprises determining the presence of SNP rs3810936, SNP rs7869487, and SNP rs6478109 in the biological sample from the patient.

In some aspects, the genotyping assay comprises determining the presence of SNP rs3810936, SNP rs7869487, SNP rs6478108, and SNP rs6478109 in the biological sample from the patient.

The genotyping assay can comprise qPCR. The genotyping assay can comprise sequencing. The genotyping assay can comprise long range sequencing or long read sequencing. The genotyping assay can be performed on a microarray. In some aspects, the genotyping assay is a fluorescence-based assay.

The biological sample can be any sample obtained from the patient. The biological sample may be a cell sample or a tissue sample. The tissue sample may be from a site of IBD inflammation. The biological sample may be an intestinal biopsy sample. The biological sample may be a blood sample. The biological sample may be buccal cells or tissue. The buccal cells or tissue can be obtained by a buccal swab. Preferably, the biological sample is blood or buccal cells or tissue.

In another aspect, the disclosure features a method of determining the risk of a patient being non-responsive to a therapeutic dose of an anti-TL1A antibody, wherein the patient has inflammatory bowel disease (IBD), the method comprising (a) performing a genotyping assay on a biological sample from the patient to determine whether the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487; and (b) identifying the patient as having a high risk of being non-responsive to a therapeutic dose of an anti-TL1A antibody if the patient is a haplotype B carrier; or (c) identifying the patient as having a low risk of being non-responsive to a therapeutic dose of an anti-TL1A antibody if the patient is a haplotype B non-carrier. In some aspects, the patient has been determined to be a haplotype B non-carrier for TNFSF15, and the method further comprises administering to the patient an effective amount of an anti-TL1A antibody. In some aspects, the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses. In some aspects, the anti-TL1A antibody is administered to the patient in a maintenance dosing regimen after completion of an induction dosing regimen, said maintenance dosing regimen comprising a plurality of individual maintenance doses separated from each other by at least 2 weeks.

In another aspect, the disclosure features a method for treating IBD in a patient, the method comprising (a) performing a genotyping assay on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487; and (b) administering to the patient an effective amount of an anti-TL1A antibody wherein the patient has been determined to be a haplotype B non-carrier. In some aspects, the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses. In some aspects, the anti-TL1A antibody is administered to the patient in a maintenance dosing regimen after completion of an induction dosing regimen, said maintenance dosing regimen comprising a plurality of individual maintenance doses separated from each other by at least 2 weeks.

In another aspect, the disclosure features a method for treating IBD in a patient, the method comprising administering to the patient an effective amount of an anti-TL1A antibody, wherein the patient has been determined to be a haplotype B non-carrier for TNFSF15 by any one of the following biomarker statuses in a biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487. In some aspects, the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses. In some aspects, the anti-TL1A antibody is administered to the patient in a maintenance dosing regimen after completion of an induction dosing regimen, said maintenance dosing regimen comprising a plurality of individual maintenance doses separated from each other by at least 2 weeks.

The individual induction doses can be administered at least daily, at least one day apart, at least 1 week apart, at least 2 weeks apart, at least 3 weeks apart, at least 4 weeks apart, at least 5 weeks apart, at least 6 weeks apart, at least 7 weeks apart, at least 8 weeks apart, at least 9 weeks apart, at least 10 weeks apart, at least 11 weeks apart, or at least 12 weeks apart. In a preferred aspect, the individual induction doses are administered one month apart. In some aspects, the individual induction dosages are administered four weeks apart.

The individual induction dose can be 1, 5, 10, 25, 50, 100, 150, 200, 250, 300, 350, 400, 450, 500, 550, 600, 650, 700, 750, 800, 850, 900, 950 or 1000 mg. In a preferred aspect, the individual induction dose is 50, 150, or 450 mg. In some aspects, each of the individual induction doses is administered at a dose of 50 mg. In some aspects, each of the individual induction doses is administered at a dose of 150 mg. In some aspects, each of the individual induction doses is administered at a dose of 450 mg.

The individual induction dosages can be administered by any means. Preferably, the individual induction dosages are administered intravenously or subcutaneously. In some aspects, each of the individual induction doses is administered subcutaneously.

In some aspects, the induction dosing regimen comprises four individual induction doses.

In some aspects, the induction dosing regimen comprises four individual induction doses administered 4 weeks apart, wherein each of the individual induction doses is administered subcutaneously at a dose of 50, 150, or 450 mg.

The individual maintenance doses can be administered at least daily, at least one day apart, at least 1 week apart, at least 2 weeks apart, at least 3 weeks apart, at least 1 month apart, at least 2 months apart, at least 3 months apart, at least 4 months apart, at least 5 months apart, or at least 6 months apart. In a preferred aspect, the individual maintenance doses are administered one month apart. In some aspects, the individual maintenance dosages are administered four weeks apart.

The individual maintenance dose can be 1, 5, 10, 25, 50, 100, 150, 200, 250, 300, 350, 400, 450, 500, 550, 600, 650, 700, 750, 800, 850, 900, 950 or 1000 mg. In a preferred aspect, the individual maintenance dose is 50, 150, or 450 mg. In some aspects, each of the individual maintenance doses is administered at a dose of 50 mg. In some aspects, each of the individual maintenance doses is administered at a dose of 150 mg. In some aspects, each of the individual maintenance doses is administered at a dose of 450 mg.

The individual maintenance dosages can be administered by any means. Preferably, the individual maintenance dosages are administered intravenously or subcutaneously. In some aspects, each of the individual maintenance doses is administered subcutaneously.

In some aspects, the maintenance dosing regimen comprises at least ten individual maintenance doses.

In some aspects, the induction dosing regimen comprises ten individual maintenance doses administered 4 weeks apart, wherein each of the individual maintenance doses is administered subcutaneously at a dose of 50, 150, or 450 mg. In some aspects, each of the individual maintenance doses is administered subcutaneously at a dose of 450 mg.

The methods provided herein can further comprise (a) determining the expression level of one or more candidate genes in a biological sample from the patient, and (b) identifying that the biological sample contains an abnormal expression level of the one of more candidate genes. The one or more candidate genes can be selected from the group consisting of SOWAHB, COLCA2, TBX20, FRZB, HOXB5, NET1, FOXD2, DESI1, PARK2, PKDREJ, IL-1 B, IL-23A, IFNG, IL-12RB1, IL-21 R, IRF4, BATF, CD80/86, HLA-DRB5/DQB1/DRB1, HLA-DRA, CD40, ICOS, MMP3, MMP7, MMP10, and CHI3L. In a preferred aspect, the one or more candidate genes are selected from the group consisting of SOWAHB, COLCA2, TBX20, FRZB, HOXB5, NET1, FOXD2, DESI1, PARK2, and PKDREJ. The expression level of the one or more candidate genes can be compared against a baseline expression level which is a) based on the expression level of the one or more candidate genes for a healthy individual who is not suffering from IBD or UC; or b) based on an estimated expression level for individuals who are non-responsive to anti-TL1A antibody treatment. The abnormal expression level of the one or more candidate genes can be at least 50% greater or lesser from the baseline level.

The disclosed methods can further comprise a) determining the expression level of one or more candidate bacterial strains in a stool sample from the patient, and b) identifying that the sample contains an increased or decreased level of the one of more candidate bacterial strains. In an aspect, the candidate bacterial strain level can be increased, and the candidate bacterial strain is selected from the group consisting of Streptococcus salivarius, Streptococcus parasanguinis, and Haemophilus parainfluenzae. In an aspect, the candidate bacterial strain level can be decreased, and the candidate bacterial strain is selected from the group consisting of Ruminococcus albus, Ruminococcus callidus, Ruminococcus bromii, Ruminococcus gnavus, and Bifidobacterium bifidum.

In another aspect, the disclosure features a method for treating inflammatory bowel disease (IBD) in a patient, the method comprising (a) performing a genotyping assay to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and (b) administering to the patient an effective amount of an anti-TL1A antibody, wherein the patient has been determined to be a haplotype B non-carrier; wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8. In some aspects, the at least two SNPs are SNP rs3810936 and SNP rs7869487. In some aspects, the assay comprises determining the presence of only two SNPs in the biological sample from the patient, wherein the two SNPs are SNP rs3810936 and SNP rs7869487.

The antibody can be any anti-TL1A antibody. Examples include, but are not limited to, afimkibart (also known as PF-06480605 or RVT-3101) from Pfizer/Roivant, PRA023 (also known as tulisokibart) from Prometheus Biosciences, and C03V from Teva Pharmaceuticals. In an aspect, the anti-TL1A antibody comprises a variable heavy chain region having the sequence shown in SEQ ID NO: 1 and a variable light chain region having the sequence shown in SEQ ID NO: 2. The anti-TL1A antibody can comprise three CDRs from the variable heavy chain region having the sequence shown in SEQ ID NO: 1 and three CDRs from the variable light chain region having the sequence shown in SEQ ID NO: 2. The anti-TL1A antibody can comprise a HCDR1 having the sequence shown in SEQ ID NO: 3 or SEQ ID NO: 68, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8 (e.g., may comprise CDR sequences according to SEQ ID NOs: 3-8 or may comprise CDR sequences according to SEQ ID NOs: 4-8 and 68). In some aspects, the anti-TL1A antibody comprises a heavy chain having the sequence shown in SEQ ID NO: 9 and a light chain having the sequence shown in SEQ ID NO: 10. In some aspects, the anti-TL1A antibody is afimkibart.

The anti-TL1A antibody can comprise sequence pairs selected from the group consisting of SEQ ID NOs: 2 and 11; SEQ ID NOs: 2 and 12; SEQ ID NOs: 2 and 13; SEQ ID NOs: 2 and 14; SEQ ID NOS: 2 and 15; SEQ ID NOs: 2 and 16; SEQ ID NOs: 2 and 17; SEQ ID NOs: 2 and 18; SEQ ID NOs: 2 and 19; SEQ ID NOs: 20 and 24; SEQ ID NOs: 21 and 25; SEQ ID NOs: 22 and 26; SEQ ID NOs: 23 and 27; SEQ ID NOs: 28 and 29; SEQ ID NOs: 30 and 31; SEQ ID NOs: 32 and 33; SEQ ID NOs: 35 and 44; SEQ ID NOs: 53 and 54; SEQ ID NOs: 61 and 62; SEQ ID NOs: 63 and 64; SEQ ID NOs: 65 and 64; SEQ ID NOs: 66 and 64; and SEQ ID NOs: 67 and 64.

In some aspects, the anti-TL1A antibody comprises three CDRs from the variable heavy chain region having the sequence shown in SEQ ID NO: 53 and three CDRs from the variable light chain region having the sequence shown in SEQ ID NO: 54. In some aspects, the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50. In some aspects, the anti-TL1A antibody comprises a variable heavy chain region having the sequence shown in SEQ ID NO: 53 and a variable light chain region having the sequence shown in SEQ ID NO: 54. In some aspects, the anti-TL1A antibody comprises a heavy chain having the sequence shown in SEQ ID NO: 35 and a light chain having the sequence shown in SEQ ID NO: 44. In some aspects, the anti-TL1A antibody is tulisokibart.

In another aspect, the disclosure features a method for treating inflammatory bowel disease (IBD) in a patient, the method comprising (a) performing a genotyping assay to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and (b) administering to the patient an anti-TL1A antibody, wherein the patient has been determined to be a haplotype B non-carrier; wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50. In some aspects, the at least two SNPs are SNP rs3810936 and SNP rs7869487. In some aspects, the assay comprises determining the presence of only two SNPs in the biological sample from the patient, wherein the two SNPs are SNP rs3810936 and SNP rs7869487. The disclosed treatment methods can further comprise treatment with an IL-23 antagonist.

In another aspect, the disclosure features a method of treating an inflammatory bowel disease (IBD) in a patient, the method comprising administering to the patient an effective amount of an anti-TNF-like ligand 1A (TL1A) antibody in a dosing regimen comprising an induction dosing regimen and a subsequent maintenance dosing regimen; wherein the induction dosing regimen comprises a plurality of individual induction doses and the maintenance dosing regimen comprising a plurality of individual maintenance doses; wherein the anti-TL1A antibody is administered subcutaneously in the maintenance phase, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In some aspects, the individual induction doses are separated from each other by at least 1 week, least 2 weeks, at least 3 weeks, or at least 4 weeks. In some aspects, the individual induction doses are separated from each other by at least 1 month. In some aspects, the individual induction doses are administered 4 weeks apart.

In some aspects, the individual induction dose is administered intravenously or subcutaneously. In some aspects, each of the individual induction doses is administered subcutaneously.

In some aspects, the individual induction dose is administered at 50, 150, or 450 mg. In some aspects, each of the individual induction doses is administered at a dose of 50 mg. In some aspects, each of the individual induction doses is administered at a dose of 150 mg. In some aspects, each of the individual induction doses is administered at a dose of 450 mg.

In some aspects, the induction dosing regimen comprises four individual induction doses.

In some aspects, the induction dosing regimen comprises four individual induction doses administered 4 weeks apart, wherein each of the individual induction doses is administered subcutaneously at a dose of 50, 150, or 450 mg.

In some aspects, the individual maintenance doses are administered at least 2 weeks, at least 3 weeks, or at least 4 weeks apart. In some aspects, the individual maintenance doses are administered at least 1 month apart. In some aspects, the individual maintenance doses are administered 4 weeks apart.

In some aspects, the individual maintenance dose is administered at 50, 150, or 450 mg. In some aspects, each of the individual maintenance doses is administered at a dose of 50 mg. In some aspects, each of the individual maintenance doses is administered at a dose of 150 mg. In some aspects, each of the individual maintenance doses is administered at a dose of 450 mg.

In some aspects, the maintenance dosing regimen comprises at least ten individual maintenance doses. In some aspects, the induction dosing regimen comprises ten individual maintenance doses administered 4 weeks apart, wherein each of the individual maintenance doses is administered subcutaneously at a dose of 50, 150, or 450 mg. In some aspects, each of the individual maintenance doses is administered subcutaneously at a dose of 450 mg.

In another aspect, the disclosure features a method of treating an inflammatory bowel disease (IBD) in a patient, the method comprising administering to the patient an effective amount of an anti-TNF-like ligand 1A (TL1A) antibody in a dosing regimen comprising an induction dosing regimen and a subsequent maintenance dosing regimen; wherein (a) the induction dosing regimen comprises subcutaneous administration of four individual induction doses of the anti-TL1A antibody at a dose of about 50 mg, 150 mg, or 450 mg, wherein the individual induction doses are administered 4 weeks apart; and (b) the maintenance dosing regimen comprises subcutaneous administration of a plurality of individual maintenance doses of the anti-TL1A antibody at a dose of about 50 mg, 150 mg, or 450 mg, wherein the individual maintenance doses are administered 4 weeks apart; and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features a method of treating an inflammatory bowel disease (IBD) in a patient, the method comprising administering to the patient an effective amount of an anti-TNF-like ligand 1A (TL1A) antibody in a dosing regimen comprising an induction dosing regimen and a subsequent maintenance dosing regimen; wherein (a) the induction dosing regimen comprises a plurality of individual induction doses, wherein the individual induction doses are separated from each other by at least 1 week, least 2 weeks, at least 3 weeks, or at least 4 weeks; and (b) the maintenance dosing regimen comprises subcutaneous administration of a plurality of individual maintenance doses of the anti-TL1A antibody at a dose of about 450 mg, wherein the individual maintenance doses are administered 4 weeks apart; and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8. In some aspects, the induction dosing regimen comprises subcutaneous administration of four individual induction doses of the anti-TL1A antibody at a dose of about 450 mg, wherein the individual induction doses are administered 4 weeks apart.

In some aspects, the first dose of the maintenance phase is administered about two weeks after administration of the final dose of the induction phase.

In some aspects, the dosing regimen has a duration of about 52 weeks.

In some aspects, the anti-TL1A antibody comprises a variable heavy chain region having the sequence shown in SEQ ID NO: 1 and a variable light chain region having the sequence shown in SEQ ID NO: 2. In some aspects, the anti-TL1A antibody comprises a heavy chain having the sequence shown in SEQ ID NO: 9 and a light chain having the sequence shown in SEQ ID NO: 10. In some aspects, the anti-TL1A antibody is afimkibart.

In some aspects, the patient has been determined to be a haplotype B non-carrier for TNFSF15.

In some aspects, the IBD is ulcerative colitis (UC). In some aspects, the UC is moderate to severe UC.

In some aspects, in a population of patients treated according to the method, the treating results in an increase in the proportion of patients who have achieved clinical remission at the end of the induction phase as compared to a reference population. In some aspects, the induction phase has a duration of about 14 weeks, and the treating results in an increase in the proportion of patients who have achieved clinical remission at Week 14. In some aspects, at least about 15% of patients in the population of patients have achieved clinical remission at Week 14. In some aspects, at least about 40% of patients in the population of patients have achieved clinical remission at Week 14.

In some aspects, in a population of patients treated according to the method, the treating results in an increase in the proportion of patients who have achieved clinical remission at the end of the maintenance phase as compared to a reference population. In some aspects, the dosing regimen has a duration of about 56 weeks, and the treating results in an increase in the proportion of patients who have achieved clinical remission at Week 56. In some aspects, at least about 20% of patients in the population of patients have achieved clinical remission at Week 56. In some aspects, at least about 55% of patients in the population of patients have achieved clinical remission at Week 56.

In some aspects, clinical remission is a total Mayo Score of ≤2, with no individual subscore >1. In some aspects, the IBD is UC and clinical remission is a modified Mayo Score (mMS)≤2 with stool frequency subscore (SFS)=0 or 1, rectal bleeding subscore (RBS)=0, and endoscopic subscore (ES)=0 or 1.

In some aspects, in a population of patients treated according to the method, the treating results in an increase in the proportion of patients who have achieved endoscopic improvement at the end of the induction phase as compared to a reference population. In some aspects, the induction phase has a duration of about 14 weeks, and the treating results in an increase in the proportion of patients who have achieved endoscopic improvement at Week 14. In some aspects, at least about 25% of patients in the population of patients have achieved endoscopic improvement at Week 14. In some aspects, at least about 50% of patients in the population of patients have achieved endoscopic improvement at Week 14.

In some aspects, in a population of patients treated according to the method, the treating results in an increase in the proportion of patients who have achieved endoscopic improvement at the end of the maintenance phase as compared to a reference population. In some aspects, the dosing regimen has a duration of about 56 weeks, and the treating results in an increase in the proportion of patients who have achieved endoscopic improvement at Week 56. In some aspects, at least about 20% of patients in the population of patients have achieved endoscopic improvement at Week 56. In some aspects, at least about 65% of patients in the population of patients have achieved endoscopic improvement at Week 56.

In some aspects, clinical remission is an endoscopic subscore of 0 or 1.

In some aspects, in a population of patients treated according to the method, the treating results in an increase in the proportion of patients who have achieved endoscopic remission, a clinical response, symptomatic remission, or deep remission at the end of the induction phase as compared to a reference population.

In some aspects, in a population of patients treated according to the method, the treating results in an increase in the proportion of patients who have achieved endoscopic remission, a clinical response, symptomatic remission, or deep remission at the end of the maintenance phase as compared to a reference population.

In some aspects, the reference population is a population of patients who have not been treated with an anti-TL1A antibody. In some aspects, the reference population is a population of patients who have been treated with a placebo.

In another aspect, the disclosure features a kit comprising an anti-TNF-like ligand 1A (TL1A) antibody and a package insert comprising instructions for using the antibody for treating an inflammatory bowel disease (IBD) in a patient in need thereof according to any one of the methods provided herein.

In another aspect, the disclosure features an anti-TL1A antibody for use in treating IBD in a patient, wherein (a) a genotyping assay is to be performed to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and (b) an effective amount of the anti-TL1A antibody is to be administered to the patient, wherein the patient has been determined to be a haplotype B non-carrier.

In another aspect, the disclosure features an anti-TL1A antibody for use in treating IBD in a patient, wherein (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; (b) the anti-TL1A antibody is to be administered to the patient in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and (c) the anti-TL1A antibody is to be administered to the patient in a subsequent maintenance dosing regimen after completion of the induction dosing regimen, said maintenance dosing regimen comprising a plurality of individual maintenance doses separated from each other by at least 2 weeks.

In another aspect, the disclosure features an anti-TL1A antibody for use in treating IBD in a patient, wherein (a) a genotyping assay is to be performed on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487; and (b) an effective amount of the anti-TL1A antibody is to be administered to the patient, wherein the patient has been determined to be a haplotype B non-carrier.

In another aspect, the disclosure features an anti-TL1A antibody for use in treating IBD in a patient, wherein an effective amount of the anti-TL1A antibody is to be administered to the patient; wherein the patient has been determined to be a haplotype B non-carrier for TNFSF15 by any one of the following biomarker statuses in a biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487.

In another aspect, the disclosure features an anti-TL1A antibody for use in treating IBD in a patient, wherein: (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and (b) an effective amount of the anti-TL1A antibody is to be administered to the patient, wherein the patient has been determined to be a haplotype B non-carrier; wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features an anti-TL1A antibody for use in treating IBD in a patient, wherein (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and (b) the anti-TL1A antibody is to be administered to the patient, wherein the patient has been determined to be a haplotype B non-carrier; wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50.

In another aspect, the disclosure features an anti-TL1A antibody for use in treating an IBD in a patient, wherein an effective amount of the anti-TL1A antibody is to be administered to the subject in a dosing regimen comprising an induction dosing regimen and a subsequent maintenance dosing regimen; wherein the induction dosing regimen comprises a plurality of individual induction doses and the maintenance dosing regimen comprising a plurality of individual maintenance doses; wherein the anti-TL1A antibody is administered subcutaneously in the maintenance phase, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features an anti-TL1A antibody for use in treating an IBD in a patient, wherein an effective amount of the anti-TL1A antibody is to be administered to the subject in a dosing regimen comprising an induction dosing regimen and a subsequent maintenance dosing regimen, wherein (a) the induction dosing regimen comprises subcutaneous administration of four individual induction doses of the anti-TL1A antibody at a dose of about 50 mg, 150 mg, or 450 mg, wherein the individual induction doses are administered 4 weeks apart; and (b) the maintenance dosing regimen comprises subcutaneous administration of a plurality of individual maintenance doses of the anti-TL1A antibody at a dose of about 50 mg, 150 mg, or 450 mg, wherein the individual maintenance doses are administered 4 weeks apart, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features an anti-TL1A antibody for use in treating an inflammatory bowel disease (IBD) in a patient, wherein an effective amount of the anti-TL1A antibody is to be administered to the subject in a dosing regimen comprising an induction dosing regimen and a subsequent maintenance dosing regimen, wherein: (a) the induction dosing regimen comprises a plurality of individual induction doses, wherein the individual induction doses are separated from each other by at least 1 week, least 2 weeks, at least 3 weeks, or at least 4 weeks; and (b) the maintenance dosing regimen comprises subcutaneous administration of a plurality of individual maintenance doses of the anti-TL1A antibody at a dose of about 450 mg, wherein the individual maintenance doses are administered 4 weeks apart, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features use of an anti-TL1A antibody in the manufacture of a medicament for treating IBD in a patient, wherein: (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and (b) an effective amount of the anti-TL1A antibody is to be administered to the patient, wherein the patient has been determined to be a haplotype B non-carrier.

In another aspect, the disclosure features use of an anti-TL1A antibody in the manufacture of a medicament for treating IBD in a patient, wherein (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; (b) the anti-TL1A antibody is to be administered to the patient in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and (c) the anti-TL1A antibody is to be administered to the patient in a subsequent maintenance dosing regimen after completion of the induction dosing regimen, said maintenance dosing regimen comprising a plurality of individual maintenance doses separated from each other by at least 2 weeks.

In another aspect, the disclosure features use of an anti-TL1A antibody in the manufacture of a medicament for treating IBD in a patient, wherein (a) a genotyping assay is to be performed on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487; and (b) an effective amount of the anti-TL1A antibody is to be administered to the patient, wherein the patient has been determined to be a haplotype B non-carrier.

In another aspect, the disclosure features use of an anti-TL1A antibody in the manufacture of a medicament for treating IBD in a patient, wherein an effective amount of the anti-TL1A antibody is to be administered to the patient, and wherein the patient has been determined to be a haplotype B non-carrier for TNFSF15 by any one of the following biomarker statuses in a biological sample from the patient: (i) the genotype of the sample is homozygous for the reference allele at rs3810936 and is homozygous for the alternate allele at rs7869487; (ii) the genotype of the sample is heterozygous for the reference allele at rs3810936 and is homozygous for the alternate allele at rs7869487; (iii) the genotype of the sample is homozygous for the alternate allele at rs3810936 and is homozygous for the reference allele at rs7869487; (iv) the genotype of the sample is homozygous for the alternate allele at rs3810936 and is heterozygous for the reference allele at rs7869487; or (v) the genotype of the sample is homozygous for the alternate allele at rs3810936 and is homozygous for the alternate allele at rs7869487.

In another aspect, the disclosure features use of an anti-TL1A antibody in the manufacture of a medicament for treating IBD in a patient, wherein: (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and (b) an effective amount of the anti-TL1A antibody is to be administered to the patient, wherein the patient has been determined to be a haplotype B non-carrier, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features use of an anti-TL1A antibody in the manufacture of a medicament for treating IBD in a patient, wherein (a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and (b) the anti-TL1A antibody is to be administered to the patient, wherein the patient has been determined to be a haplotype B non-carrier, and wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50.

In another aspect, the disclosure features use of an anti-TL1A antibody in the manufacture of a medicament for treating an IBD in a patient, wherein an effective amount of the anti-TL1A antibody is to be administered to the subject in a dosing regimen comprising an induction dosing regimen and a subsequent maintenance dosing regimen; wherein the induction dosing regimen comprises a plurality of individual induction doses and the maintenance dosing regimen comprising a plurality of individual maintenance doses; wherein the anti-TL1A antibody is administered subcutaneously in the maintenance phase, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features use of an anti-TL1A antibody in the manufacture of a medicament for treating an IBD in a patient, wherein an effective amount of the anti-TL1A antibody is to be administered to the subject in a dosing regimen comprising an induction dosing regimen and a subsequent maintenance dosing regimen, wherein (a) the induction dosing regimen comprises subcutaneous administration of four individual induction doses of the anti-TL1A antibody at a dose of about 50 mg, 150 mg, or 450 mg, wherein the individual induction doses are administered 4 weeks apart; and (b) the maintenance dosing regimen comprises subcutaneous administration of a plurality of individual maintenance doses of the anti-TL1A antibody at a dose of about 50 mg, 150 mg, or 450 mg, wherein the individual maintenance doses are administered 4 weeks apart, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In another aspect, the disclosure features use of an anti-TL1A antibody in the manufacture of a medicament for treating an inflammatory bowel disease (IBD) in a patient, wherein an effective amount of the anti-TL1A antibody is to be administered to the subject in a dosing regimen comprising an induction dosing regimen and a subsequent maintenance dosing regimen, wherein (a) the induction dosing regimen comprises a plurality of individual induction doses, wherein the individual induction doses are separated from each other by at least 1 week, least 2 weeks, at least 3 weeks, or at least 4 weeks; and (b) the maintenance dosing regimen comprises subcutaneous administration of a plurality of individual maintenance doses of the anti-TL1A antibody at a dose of about 450 mg, wherein the individual maintenance doses are administered 4 weeks apart, and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In some aspects, the patient is a human.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed disclosure. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIG. 1 is an illustration showing the location of five single nucleotide polymorphisms (SNPs) in the TNFSF15 gene loci.

FIG. 2 is a schematic diagram showing the TL1A 2-SNP Test workflow. qPCR=quantitative PCR.

FIG. 3 is a schematic diagram showing the design of the TUSCANY-2 study. Following a screening period, subjects received four monthly doses of afimkibart in a 12-week induction period (50 mg, 150 mg, or 450 mg), and then received monthly doses of afimkibart in a maintenance period (50 mg, 150 mg, or 450 mg). SC=subcutaneous. Endpoints assessed at Weeks 14 and 56 were clinical remission and endoscopic improvement.

FIG. 4A is a bar graph showing the percentage of patients who received at least one dose of the indicated dose of afimkibart or placebo in the induction phase of the TUSCANY-2 study and experienced clinical remission (defined as modified Mayo Score (mMS)≤2 with stool frequency subscore (SFS)=0 or 1, rectal bleeding subscore (RBS)=0, and endoscopic subscore (ES)=0 or 1) at Week 14. Unadjusted outcome rates with 90% confidence intervals are shown.

FIG. 4B is a bar graph showing the percentage of patients who received at least one dose of the indicated dose of afimkibart or placebo in the induction phase of the TUSCANY-2 study and experienced endoscopic improvement at Week 14. Unadjusted outcome rates with 90% confidence intervals are shown.

FIG. 5 is a bar graph showing the percentage of patients who received at least one dose of the indicated dose of afimkibart or placebo in the maintenance phase of the TUSCANY-2 study and experienced clinical remission (defined as modified Mayo Score (mMS)≤2 with stool frequency subscore (SFS)=0 or 1, rectal bleeding subscore (RBS)=0, and endoscopic subscore (ES)=0 or 1) at Week 14 or Week 56. Unadjusted outcome rates with 90% confidence intervals are shown.

FIG. 6 is a bar graph showing the percentage of patients who received at least one dose of the indicated dose of afimkibart or placebo in the maintenance phase of the TUSCANY-2 study and experienced endoscopic improvement at Week 14 or Week 56. Unadjusted outcome rates with 90% confidence intervals are shown.

FIG. 7A is a set of bar graphs showing the percentage of patients treated with afimkibart or placebo in the induction phase of the TUSCANY-2 study who experienced clinical remission (according to modified Mayo Score (mMS)) at Week 14. Pbo: placebo. Biomarker-positive: TNFSF15 haplotype B non-carrier.

FIG. 7B is a set of bar graphs showing the percentage of patients treated with afimkibart or placebo in the induction phase of the TUSCANY-2 study who experienced endoscopic improvement at Week 14. Pbo: placebo. Biomarker-positive: TNFSF15 haplotype B non-carrier.

FIG. 8A is a set of bar graphs showing the percentage of patients treated with afimkibart or placebo in the induction phase of the TUSCANY-2 study who experienced clinical remission at Week 14. IR: intolerance, inadequate response, or loss of response to prior advanced therapy (anti-tumor necrosis factor (TNF) agent, anti-integrin agent, anti-IL-12/23 agent, and/or Janus kinase (JAK) inhibitor). Not IR: naïve to failure of an advanced therapy.

FIG. 8B is a set of bar graphs showing the percentage of patients treated with afimkibart or placebo in the induction phase of the TUSCANY-2 study who experienced endoscopic improvement at Week 14. IR: intolerance, inadequate response, or loss of response to prior advanced therapy (anti-tumor necrosis factor (TNF) agent, anti-integrin agent, anti-IL-12/23 agent, and/or Janus kinase (JAK) inhibitor). Not IR: naïve to failure of an advanced therapy.

FIG. 9A is a set of bar graphs showing the percentage of patients treated with afimkibart or placebo in the induction phase of the TUSCANY-2 study who experienced clinical remission at Week 14. IR: intolerance, inadequate response, or loss of response to a previously administered therapy comprising an anti-TNF agent. Not IR: naïve to failure of a therapy comprising an anti-TNF agent.

FIG. 9B is a set of bar graphs showing the percentage of patients treated with afimkibart or placebo in the induction phase of the TUSCANY-2 study who experienced endoscopic improvement at Week 14. IR: intolerance, inadequate response, or loss of response to a previously administered therapy comprising an anti-TNF agent. Not IR: naïve to failure of a therapy comprising an anti-TNF agent.

FIG. 10 is a set of bar graphs showing the percentage of biomarker-evaluable, biomarker-positive, and biomarker-negative patients treated with afimkibart or placebo in the induction phase of the TUSCANY-2 study who experienced clinical remission (left) or endoscopic improvement (right) at Week 14. Biomarker-positive: TNFSF15 haplotype B non-carrier. Biomarker-negative: TNFSF15 haplotype B carrier. Unadjusted outcome rates with 90% confidence intervals are shown.

### DETAILED DESCRIPTION OF THE INVENTION

### I. Methods of Clinical Management and Treatment

A major goal of emerging therapies is to utilize genomics and transcriptomics in participants at baseline that may help to stratify and maximize therapeutic efficacy while minimizing risk. The present disclosure provides methods to optimize clinical management using anti-TL1A treatment. In some embodiments, the methods provided herein optimize clinical management using anti-TL1A to treat inflammatory bowel disease, such as Crohn's disease (CD) or ulcerative colitis (UC), including any one of the treatment methods described in Section II below and/or known in the art, for example, as described in U.S. Pat. Nos. 10,822,422, 10,138,296, 11,292,848, 9,068,003, 10,590,201, 9,896,511, U.S. Pat. Nos. 8,642,741, 9,290,576, 8,728,482, 8,263,743, 9,416,185, 10,322,174, 10,689,439, 11,440,954, 11,220,549, 11,767,364, U.S. Patent Pub. No. 2023/0235070, and U.S. Patent Pub. No. US20240059799A1, the contents of each of which are incorporated herein by reference in its entirety. Any of the methods described in this section (i.e., Section I) may be used for treatment using any of the anti-TL1A antibodies described in Section IV below.

### A. TNFSF15 Haplotypes and Risk of Non-Responsiveness

In some aspects, any of the methods, uses, or compositions for use provided herein comprise determining the TNFSF15 haplotype of a patient (e.g., comprise determining whether a patient is (a) a TL1A haplotype B carrier or (b) a TL1A haplotype B non-carrier). The methods, uses, or compositions for use may further include making a treatment decision on the basis of the patient's TNFSF15 haplotype (e.g., a decision to administer an anti-TL1A antibody (e.g., an anti-TL1A antibody provided in Section IV herein, e.g., afimkibart, tulisokibart, or TEV-48574) or an anti-DR3 antibody to the patient).

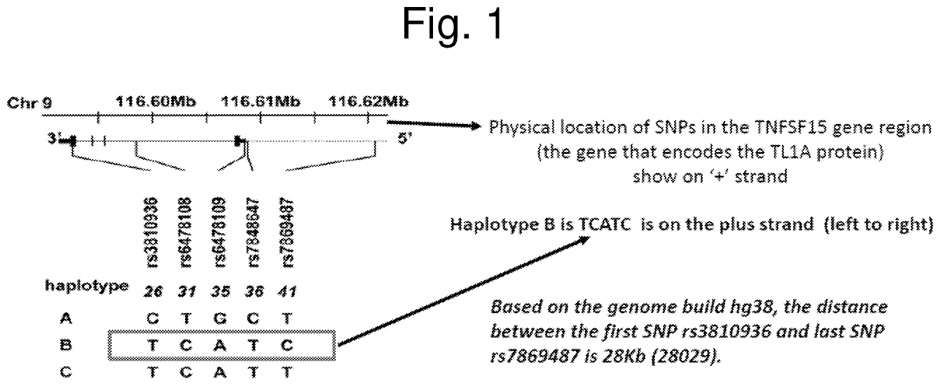

TNFSF15 haplotype B is defined as a TCATC on the plus strand at the five single nucleotide polymorphism (SNP) sites rs3810936, rs6478108, rs6478109, rs7848647, and rs7869487 in the TL1A gene loci of chromosome 9 that span a 28 kb region surrounding TNFSF15, the gene encoding TL1A (FIG. 1). TNFSF15 haplotype B and other TNFSF15 haplotypes are shown in Table 1.

TNFSF15 haplotype B has been associated with disease susceptibility and/or TL1A expression in Japanese and European cohorts. Data suggest Jewish Crohn's disease (CD) patients that are haplotype B carriers have more severe disease. Phase 2a and 2b studies show differential effects between haplotype B carrier and non-carrier subpopulation. Data from internal UC studies support specificity of haplotype B status in predicting response to anti-TL1A therapy.

A TNFSF15 haplotype B non-carrier is any individual who does not carry TCATC on the plus strand of either their maternal or paternal chromosome 9. Haplotype B non-carriers demonstrate moderate TL1A tissue expression and increased response to anti-TL1A therapy.

TL1A haplotype determination can be accomplished using a 2-SNP assay. The two SNPs can be any two SNPs selected from rs3810936, rs6478108, rs6478109, rs7848647, and rs7869487 to differentiate haplotype B from non-haplotype B (carrier vs non-carrier). In one aspect, the two SNPs are rs3810936 and rs7869487. In a preferred aspect, SNPs rs3810936 and rs7869487 can differentiate haplotype B from non-haplotype B (carrier vs. non-carrier). Determination can proceed as follows: Step 1: Assess SNP rs7869487 in a biological sample from a patient. If (T/T), then the patient is identified as non-haplotype B carrier. If (C/C) or (C/T), proceed to Step 2. Step 2: Assess SNP rs3810936. If (C/C), the patient is identified as non-haplotype B carrier. If (T/T) or (C/T), the patient is identified as haplotype B carrier (>99% probability).

TABLE 1
TNFSF15 Haplotype B Single Nucleotide Polymorphisms

| Haplotype | rs3810936 | rs6478108 | rs6478109 | rs7848647 | rs7869487 |
|---|---|---|---|---|---|
| A | C | T | G | C | T |
| B | T | C | A | T | C |
| C | T | C | A | T | T |
| D | T | T | G | C | T |
| E | C | C | A | T | C |
| F | C | C | G | C | C |

The average misclassification rate with the 2-SNP analysis above is extremely low and will readily permit use in clinical assays. 41% of American subjects (of multiple ancestral backgrounds) and subjects of European ancestry (combined) were haplotype B carriers with an average misclassification rate of ~0.5%. Subjects of European ancestry had a higher frequency of haplotype B carriers compared to American subjects (of multiple ancestral backgrounds). 49.2% of subjects of East Asian and South Asian ancestry (combined) were haplotype B carriers with an average misclassification rate of ~1.6%. Subjects of East Asian ancestry are the largest population of haplotype B carriers (~66%). 8.32% of subjects of African ancestry were haplotype B carriers, no misclassifications were identified in this population. In an evaluation of the 2-SNP analysis in phase 2 studies, 62/192 samples were heterozygous or homozygous for the minor allele frequency (MAF) at rs7869487 or rs3810936. All 62 subjects were considered haplotype B positive—0% mischaracterization.

### Methods of Determining Risk of Non-Responsiveness

The present disclosure provides methods of determining the risk of a patient being non-responsive to a therapeutic dose of an anti-TNF-like ligand 1A (TL1A) antibody (e.g., an anti-TL1A antibody provided in Section IV herein, e.g., afimkibart, tulisokibart, or TEV-48574) or an anti-DR3 antibody comprising (a) performing a genotyping assay on a biological sample from the patient to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient, and (b) determining the risk of being non-responsive to the therapeutic dose of an anti-TL1A antibody or an anti-DR3 antibody, wherein: (i) the risk is higher in a haplotype B carrier patient than in a haplotype B non-carrier patient; (ii) the risk of being non-responsive is lower in a haplotype B non-carrier patient than in a haplotype B carrier patient; and/or (iii) the likelihood of being responsive is higher in a haplotype B non-carrier patient than in a haplotype B carrier patient. Determining the presence of the at least two SNPs determines that the patient is a haplotype B carrier. The patient can have inflammatory bowel disease (IBD). In a preferred aspect, the at least two SNPs are SNP rs3810936 and SNP rs7869487.

In another aspect, the present disclosure provides a method of determining the risk of a patient being non-responsive to a therapeutic dose of an anti-TL1A antibody (e.g., an anti-TL1A antibody provided in Section IV herein, e.g., afimkibart, tulisokibart, or TEV-48574) or an anti-DR3 antibody comprising (a) performing a genotyping assay on a biological sample from the patient to determine whether the patient is a haplotype B carrier or a haplotype B non-carrier for TNFSF15, the assay comprising determining the presence of at least two single nucleotide polymorphisms (SNPs) comprising r3810936 and rs7869487 in a biological sample from the patient, and (b) determining the risk of being non-responsive to the therapeutic dose of an anti-TL1A antibody or an anti-DR3 antibody, wherein: (i) the risk is higher in a haplotype B carrier patient than in a haplotype B non-carrier patient; (ii) the risk of being non-responsive is lower in a haplotype B non-carrier patient than in a haplotype B carrier patient; and/or (iii) the likelihood of being responsive is higher in a haplotype B non-carrier patient than in a haplotype B carrier patient.

In another aspect, the present disclosure provides a method of determining the risk of a patient being non-responsive to a therapeutic dose of an anti-TL1A antibody (e.g., an anti-TL1A antibody provided in Section IV herein, e.g., afimkibart, tulisokibart, or TEV-48574) or an anti-DR3 antibody comprising (a) performing a genotyping assay on a biological sample from the patient to determine whether the patient is a haplotype B carrier or a haplotype B non-carrier for TNFSF15, the assay comprising determining the presence of the two single nucleotide polymorphisms (SNPs) r3810936 and rs7869487 in a biological sample from the patient, and (b) determining the risk of being non-responsive to the therapeutic dose of an anti-TL1A antibody or an anti-DR3 antibody, wherein: (i) the risk is higher in a haplotype B carrier patient than in a haplotype B non-carrier patient; (ii) the risk of being non-responsive is lower in a haplotype B non-carrier patient than in a haplotype B carrier patient; and/or (iii) the likelihood of being responsive is higher in a haplotype B non-carrier patient than in a haplotype B carrier patient. In one aspect, the present disclosure provides a method of determining the risk of a patient being non-responsive to a therapeutic dose of an anti-TL1A antibody or an anti-DR3 antibody, the method comprising (a) performing a genotyping assay on a biological sample from the patient to determine whether the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient:
(i) the genotype of the sample is homozygous for the reference allele at rs3810936 (i.e., the sample is T/T at rs3810936) and is homozygous for the alternate allele at rs7869487 (i.e., the sample is T/T at rs7869487);
(ii) the genotype of the sample is heterozygous for the reference allele at rs3810936 (e.g., the sample is C/T at rs3810936) and is homozygous for the alternate allele at rs7869487 (i.e., the sample is T/T at rs7869487);
(iii) the genotype of the sample is homozygous for the alternate allele at rs3810936 (i.e., the sample is C/C at rs3810936) and is homozygous for the reference allele at rs7869487 (i.e., the sample is C/C at rs7869487);
(iv) the genotype of the sample is homozygous for the alternate allele at rs3810936 (i.e., the sample is C/C at rs3810936) and is heterozygous for the reference allele at rs7869487 (e.g., the sample is T/C at rs7869487); or
(v) the genotype of the sample is homozygous for the alternate allele at rs3810936 (i.e., the sample is C/C at rs3810936) and is homozygous for the alternate allele at rs7869487 (i.e., the sample is T/T at rs7869487); and
(b) identifying the patient as having a high risk of being non-responsive to a therapeutic dose of an anti-TL1A antibody or an anti-DR3 antibody if the patient is a haplotype B carrier; or (c) identifying the patient as having a low risk of being non-responsive to a therapeutic dose of an anti-TL1A antibody or an anti-DR3 antibody if the patient is a haplotype B non-carrier.

### Methods of Treating IBD Based on TNFSF15 Haplotype

In another aspect, the disclosure provides a method for treating inflammatory bowel disease (IBD) in a patient, the method comprising (a) performing a genotyping assay to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and (b) administering to the patient an effective amount of an anti-TL1A antibody (e.g., an anti-TL1A antibody provided in Section IV herein, e.g., afimkibart, tulisokibart, or TEV-48574) or an anti-DR3 antibody, wherein the patient has been determined to be a haplotype B non-carrier.

In some embodiments, the anti-TL1A antibody is administered to the patient in an induction dosing regimen (e.g., is administered to the patient in an induction dosing regimen sufficient to improve signs and symptoms of IBD by at least 12 weeks after the start of treatment with the anti-TL1A antibody, said induction dosing regimen comprising a plurality of individual induction doses).

In some embodiments, the anti-TL1A antibody is administered to the patient in a maintenance dosing regimen following an induction dosing regimen (e.g., is administered to the patient in a maintenance dosing regimen after completion of the induction dosing regimen, said maintenance dosing regimen comprising a plurality of individual maintenance doses separated from each other by at least 2 weeks). Exemplary induction and maintenance dosing regimens are provided in Section II, below.

Accordingly, in some aspects, the disclosure provides a method for treating IBD in a patient, the method comprising (a) performing a genotyping assay to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; (b) administering to the patient an anti-TL1A antibody in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and (c) administering to the patient a subsequent maintenance dosing regimen after completion of the induction dosing regimen, said maintenance dosing regimen comprising a plurality of individual maintenance doses separated from each other by at least 2 weeks.

In some embodiments, the at least two SNPs are SNP rs3810936 and SNP rs7869487. In some embodiments, the assay comprises determining the presence of only two SNPs in the biological sample from the patient, wherein the two SNPs are SNP rs3810936 and SNP rs7869487. At the position of SNP rs3810936, the wild-type nucleotide (“reference allele”) is “T”, and the alternate allele is “C”. At the position of SNP rs7869487, the wild-type nucleotide (“reference allele”) is “C”, and the alternate allele is “T”.

In some embodiments, a haplotype B non-carrier for TNFSF15 is identified by any one of the following biomarker statuses in the biological sample from the patient:
(i) the genotype of the sample is homozygous for the reference allele at rs3810936 (i.e., the sample is T/T at rs3810936) and is homozygous for the alternate allele at rs7869487 (i.e., the sample is T/T at rs7869487);
(ii) the genotype of the sample is heterozygous for the reference allele at rs3810936 (e.g., the sample is C/T at rs3810936) and is homozygous for the alternate allele at rs7869487 (i.e., the sample is T/T at rs7869487);
(iii) the genotype of the sample is homozygous for the alternate allele at rs3810936 (i.e., the sample is C/C at rs3810936) and is homozygous for the reference allele at rs7869487 (i.e., the sample is C/C at rs7869487);
(iv) the genotype of the sample is homozygous for the alternate allele at rs3810936 (i.e., the sample is C/C at rs3810936) and is heterozygous for the reference allele at rs7869487 (e.g., the sample is T/C at rs7869487); or
(v) the genotype of the sample is homozygous for the alternate allele at rs3810936 (i.e., the sample is C/C at rs3810936) and is homozygous for the alternate allele at rs7869487 (i.e., the sample is T/T at rs7869487).

In some embodiments, the at least two SNPs comprise SNP rs3810936, SNP rs7869487, and SNP rs7848647. In some embodiments, the assay comprises determining the presence of only three SNPs in the biological sample from the patient, wherein the three SNPs are SNP rs3810936, SNP rs7869487, and SNP rs7848647. In some embodiments, the assay comprises determining the presence of only three SNPs in the biological sample from the patient, wherein the three SNPs are SNP rs3810936, SNP rs7869487, and SNP rs6478109.

In some embodiments, the at least two SNPs comprise SNP rs3810936, SNP rs7869487, SNP rs6478108, and SNP rs6478109. In some embodiments, the assay comprises determining the presence of only four SNPs in the biological sample from the patient, wherein the four SNPs are SNP rs3810936, SNP rs7869487, SNP rs6478108, and SNP rs6478109.

Thus, in some aspects, the disclosure provides a method for treating IBD in a patient, the method comprising (a) performing a genotyping assay on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient:
(i) the genotype of the sample is homozygous for the reference allele at rs3810936 (i.e., the sample is T/T at rs3810936) and is homozygous for the alternate allele at rs7869487 (i.e., the sample is T/T at rs7869487);
(ii) the genotype of the sample is heterozygous for the reference allele at rs3810936 (e.g., the sample is C/T at rs3810936) and is homozygous for the alternate allele at rs7869487 (i.e., the sample is T/T at rs7869487);
(iii) the genotype of the sample is homozygous for the alternate allele at rs3810936 (i.e., the sample is C/C at rs3810936) and is homozygous for the reference allele at rs7869487 (i.e., the sample is C/C at rs7869487);
(iv) the genotype of the sample is homozygous for the alternate allele at rs3810936 (i.e., the sample is C/C at rs3810936) and is heterozygous for the reference allele at rs7869487 (e.g., the sample is T/C at rs7869487); or
(v) the genotype of the sample is homozygous for the alternate allele at rs3810936 (i.e., the sample is C/C at rs3810936) and is homozygous for the alternate allele at rs7869487 (i.e., the sample is T/T at rs7869487); and
(b) administering an anti-TL1A antibody or an anti-DR3 antibody to a patient who has been determined to be a haplotype B non-carrier. In some embodiments, the anti-TL1A antibody is administered to the patient in an induction dosing regimen (e.g., is administered to the patient in an induction dosing regimen sufficient to improve signs and symptoms of IBD by at least 12 weeks after the start of treatment with the anti-TL1A or anti-DR3 antibody, said induction dosing regimen comprising a plurality of individual induction doses). In some embodiments, the anti-TL1A antibody is further administered to the patient in a maintenance dosing regimen following an induction dosing regimen (e.g., is administered to the patient in a subsequent maintenance dosing regimen after completion of an induction dosing regimen, said maintenance dosing regimen comprising a plurality of individual maintenance doses separated from each other by at least 2 weeks).

In another aspect, the disclosure provides a method for treating IBD in a patient, the method comprising (a) administering to the patient an effective amount of an anti-TL1A antibody in an induction dosing regimen (e.g., an induction dosing regimen sufficient to improve signs and symptoms of IBD by at least 12 weeks after the start of treatment with the anti-TL1A antibody, said induction dosing regimen comprising a plurality of individual induction doses); and (b) administering to the patient an effective amount of an anti-TL1A antibody in a subsequent maintenance dosing regimen after completion of the induction dosing regimen, e.g., a maintenance dosing regimen comprising a plurality of individual maintenance doses separated from each other by at least 2 weeks; wherein the patient has been determined to be a haplotype B non-carrier for TNFSF15, wherein a haplotype B non-carrier for TNFSF15 is identified by any one of the following biomarker statuses in a biological sample from the patient:
(i) the genotype of the sample is homozygous for the reference allele at rs3810936 (i.e., the sample is T/T at rs3810936) and is homozygous for the alternate allele at rs7869487 (i.e., the sample is T/T at rs7869487);
(ii) the genotype of the sample is heterozygous for the reference allele at rs3810936 (e.g., the sample is C/T at rs3810936) and is homozygous for the alternate allele at rs7869487 (i.e., the sample is T/T at rs7869487);
(iii) the genotype of the sample is homozygous for the alternate allele at rs3810936 (i.e., the sample is C/C at rs3810936) and is homozygous for the reference allele at rs7869487 (i.e., the sample is C/C at rs7869487);
(iv) the genotype of the sample is homozygous for the alternate allele at rs3810936 (i.e., the sample is C/C at rs3810936) and is heterozygous for the reference allele at rs7869487 (e.g., the sample is T/C at rs7869487); or
the genotype of the sample is homozygous for the alternate allele at rs3810936 (i.e., the sample is C/C at rs3810936) and is homozygous for the alternate allele at rs7869487 (i.e., the sample is T/T at rs7869487).

In some embodiments of any of the above aspects, the anti-TL1A antibody comprises a HCDR1 (CDR H1) having the sequence shown in SEQ ID NO: 3, a HCDR2 (CDR H2) having the sequence shown in SEQ ID NO: 4, a HCDR3 (CDR H3) having the sequence shown in SEQ ID NO: 5, a LCDR1 (CDR L1) having the sequence shown in SEQ ID NO: 6, a LCDR2 (CDR L2) having the sequence shown in SEQ ID NO: 7, and a LCDR3 (CDR L3) having the sequence shown in SEQ ID NO: 8. In some embodiments of any of the above aspects, the anti-TL1A antibody comprises a HCDR1 (CDR H1) having the sequence shown in SEQ ID NO: 68, a HCDR2 (CDR H2) having the sequence shown in SEQ ID NO: 4, a HCDR3 (CDR H3) having the sequence shown in SEQ ID NO: 5, a LCDR1 (CDR L1) having the sequence shown in SEQ ID NO: 6, a LCDR2 (CDR L2) having the sequence shown in SEQ ID NO: 7, and a LCDR3 (CDR L3) having the sequence shown in SEQ ID NO: 8. In some embodiments, the anti-TL1A antibody is afimkibart.

In some embodiments of any of the above aspects, the anti-TL1A antibody comprises a CDR-H1 having the sequence shown in SEQ ID NO: 37, a CDR-H2 having the sequence shown in SEQ ID NO: 39, a CDR-H3 having the sequence shown in SEQ ID NO: 41, a CDR-L1 having the sequence shown in SEQ ID NO: 46, a CDR-L2 having the sequence shown in SEQ ID NO: 48, and a CDR-L3 having the sequence shown in SEQ ID NO: 50. In some embodiments, the anti-TL1A antibody is tulisokibart.

In some embodiments of any of the above aspects, the anti-TL1A antibody has the HVR sequences of TEV-48574. In some embodiments, the anti-TL1A antibody is TEV-48574.

In some embodiments of any of the above aspects, the IBD is ulcerative colitis. In some embodiments, the UC is moderate to severe UC.

### Assays for Determining TNFSF15 Haplotype

Any appropriate method may be used to determine whether a patient is a haplotype B carrier or a haplotype B non-carrier for TNFSF15. For example, any appropriate method may be used to determine a patient's genotype at one or more of the positions of at least two SNPs selected from rs3810936, rs6478108, rs6478109, rs7848647, and rs7869487 in a biological sample from the patient. Exemplary methods for determining the TNFSF15 haplotype of a patient are provided herein.

In some embodiments, the genotyping assay used in any one of the methods provided herein comprises use of quantitative PCR (qPCR).

In some embodiments, the genotyping assay comprises sequencing (e.g., comprises DNA sequencing). In some embodiments, the genotyping assay comprises long-read sequencing.

In some embodiments, the genotyping assay is a fluorescence-based assay. In some embodiments, the genotyping assay is a fluorescence-based qPCR assay, such as a TAQMAN™ qPCR assay.

The biological sample may be any appropriate sample comprising genomic DNA. In some embodiments, the biological sample is blood. In some embodiments, the biological sample is or comprises buccal cells.

### B. Expression Level of Candidate Genes

In some aspects, any of the methods, uses, or compositions for use provided herein comprise determining the expression of one or more candidate genes (as described below) in a sample from a patient. The methods, uses, or compositions for use may further include making a treatment decision on the basis of the patient's gene expression.

In some aspects of the disclosure, the method further comprises the steps of: a) determining the expression level of one or more candidate genes in a sample from the patient, b) identifying that the sample contains an abnormal expression level of the one of more candidate gene, c) administering the induction dosing regimen or individual induction dose of the anti-TL1A antibody to a patient.

Determining whether the patient has an abnormal expression level of one or more candidate genes may be by obtaining or having obtained a sample from the patient. The sample may be a tissue sample. The sample may be a tissue sample from a site of IBD inflammation. The sample may be a peripheral blood sample. The sample may be an intestinal biopsy sample.

The method may further comprise performing or having performed an assay on the sample to determine if the patient expresses abnormal levels of the one or more candidate genes.

In some aspects, if the sample contains abnormal levels of the one or more candidate gene then the method provides for the further step of administering an induction dosing regimen or induction dose of the anti-TL1A antibody to the patient.

In some aspects, the risk of the patient being non-responsive to an induction dosing regimen or individual induction dose of an anti-TL1A antibody is lower in a patient with abnormal levels of the one or more candidate genes.

In some aspects, the disclosure provides a method for treating inflammatory bowel disease (IBD) in a patient, the method comprising the steps of: a) determining the expression level of one or more candidate genes in a sample from the patient, b) identifying that the sample contains an abnormal expression level of the one of more candidate gene, and c) administering an induction dosing regimen or individual induction dose of an anti-TNF-like ligand 1A (TL1A) antibody to the patient.

The one or more candidate genes may be selected from the group consisting of IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L. The one or more candidate genes may be selected from the group consisting of IL-1B, IL-23A, IFNG, IL-12RB1, IL-21R, IRF4, and BATF. The one or more candidate genes may be selected from the group consisting of CD80/86, HLA-DRB5/DQB1/DRB1, HLA-DRA, CD40, and ICOS. The one or more candidate genes may be selected from the group consisting of MMP3, MMP7, MMP10 and CHI3L.

The one or more candidate genes may be selected from the group consisting of SOWAHB, COLCA2, TBX20, FRZB, HOXB5, NET1, FOXD2, DESI1, PARK2, PKDREJ, IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L.

The one or more candidate genes may be selected from the group consisting of SOWAHB, COLCA2, TBX20, FRZB, HOXB5, NET1, FOXD2, DESI1, PARK2, and PKDREJ. The one or more candidate genes may comprise SOWAHB. The one or more candidate genes may comprise SOWAHB, and at least one or more candidate genes selected from the group consisting of COLCA2, TBX20, FRZB, HOXB5, NET1, FOXD2, DESI1, PARK2, and PKDREJ. The one or more candidate genes may comprise SOWHAB and COLCA2, and at least one or more candidate genes selected from the group consisting of SOWAHB, COLCA2, TBX20, FRZB, HOXB5, NET1, FOXD2, DESI1, PARK2, and PKDREJ. The one or more candidate genes may comprise SOWAHB, COLCA2, and TBX20 and at least one or more candidate genes selected from the group consisting of FRZB, HOXB5, NET1, FOXD2, DESI1, PARK2, and PKDREJ.

In some aspects, the method provides determining the expression level of 2, or 3, or 4, or 5, or 6, or 7, or 8 or 9, or 10 of the candidate genes selected from the group consisting of SOWAHB, COLCA2, TBX20, FRZB, HOXB5, NET1, FOXD2, DESI1, PARK2, and PKDREJ.

The abnormal expression level of the one or more candidate genes may be based on the one or more candidate genes' level of mRNA or expressed protein.

The abnormal expression level of the one or more candidate genes may be based on the one or more candidate genes' mRNA levels.

The expression level of the one or more candidate gene may be compared against a baseline expression level which is based on the expression level of the one or more candidate gene for a healthy individual who is not suffering from IBD or UC.

The expression level of the one or more candidate genes may be compared against a baseline expression level which is based on an estimated expression level for individuals who are non-responsive to anti-TL1A antibody treatment.

The abnormal expression level of the one or more candidate gene may be at least 50% greater or lesser from the baseline level. The abnormal expression level of the one or more candidate gene may be at least 2-fold, 5-fold, 10-fold, 50-fold, 100-fold, 500-fold, or 1000-fold greater or lesser from the baseline level.

In some aspects, when the one or more of the candidate genes is selected from the group consisting of SOWAHB, COLCA2, FRZB, HOXB5, NET1, FOXD2, PARK2, and PKDREJ, then the abnormal expression level may be an elevated level. In some aspects, when the one or more of the candidate genes is selected from the group consisting of TBX20 and DESI1, then the abnormal expression level is a decreased level.

### C. Levels of Bacterial Strains

In some aspects, any of the methods, uses, or compositions for use provided herein comprise determining the level of one or more bacterial strains (as described below) in a stool sample from a patient. The methods, uses, or compositions for use may further include making a treatment decision on the basis of levels of the bacterial strains in the sample.

In some aspects, the disclosure comprises the steps of: a) determining the level of one or more candidate bacterial strains in a stool sample from the patient, b) identifying that the stool sample contains an elevated level of the one of more candidate bacterial strains, c) administering the induction dose of the anti-TL1A antibody to a patient.

In some aspects, the candidate bacterial strain is selected from the group consisting of Streptococcus salivarius, Streptococcus parasanguinis, and Haemophilus parainfluenzae.

In some aspects, the disclosure comprises the steps of: a) determining the level of one or more candidate bacterial strains in a stool sample from the patient, b) identifying that the stool sample contains a decreased level of the one of more candidate bacterial strains, c) administering the induction dose of the anti-TL1A antibody to a patient.

In some aspects, the candidate bacterial strain is selected from the group consisting of Ruminococcus albus, Ruminococcus callidus, Ruminococcus bromii, Ruminococcus gnavus, and Bifidobacterium bifidum.

In some aspects, the level of the one or more candidate bacterial strains is compared against a baseline bacterial level which is based on the level of the one or more candidate bacterial strain for a healthy individual who is not suffering from IBD or UC. In some aspects, the level of the one or more candidate bacterial strains is compared against a baseline bacterial level which is based on an estimated level of those candidate bacterial strains for individuals who are non-responsive to anti-TL1A antibody treatment.

In some aspects, the level of the one or more candidate bacterial strains is at least greater or lesser from the baseline bacterial level by at least 50%, 2-fold, 5-fold, 10-fold, 20-fold, 50-fold, 100-fold, 500-fold, or 1000-fold.

In some aspects, the disclosure further comprises treatment with an IL-23 antagonist.

D. Methods of Identifying a Patient as Likely to Benefit from Treatment Based on Abnormal Gene Expression

The disclosure provides a method for identifying a patient having inflammatory bowel disease as being likely to benefit from initial or continued treatment with anti-TL1A antibody treatment, and optionally treating said patient, wherein said method comprises: (a) identifying a patient as containing an abnormal level of one or more candidate genes, selected from the group consisting of SOWAHB, COLCA2, TBX20, FRZB, HOXB5, NET1, FOXD2, DESI1, PARK2, PKDREJ, IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L; and optionally further includes (b) administering or having administered to said patient an anti-TL1A antibody under conditions wherein one or more selected from the group consisting of inflammatory macrophages, TH17, ILC3, 0X40, OX40L, IFNy, ILC2, IL-13, MMP, tissue remodeling, fibrosis, the intestinal population of S. salivarius, the intestinal population of S. parasanguinis, and the intestinal population of H. parainfluenzae in said patient is reduced after said administering.

### II. Methods of Treating Inflammatory Bowel Disease with Anti-TL1A Antibodies

In some aspects, the disclosure provides a method of treating an inflammatory bowel disease (IBD) (e.g., ulcerative colitis (UC) or Crohn's disease (CD)) in a patient (e.g., a human), the method comprising administering to the patient an effective amount of an anti-TNF-like ligand 1A (TL1A) antibody (e.g., an anti-TL1A antibody provided in Section IV herein) in a dosing regimen comprising an induction phase (induction dosing regimen) and a subsequent maintenance phase (maintenance dosing regimen), wherein the induction dosing regimen comprises a plurality of individual induction doses and the maintenance dosing regimen comprising a plurality of individual maintenance doses, and wherein the anti-TL1A antibody is administered subcutaneously in the maintenance phase. In some embodiments, the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8. In some embodiments, the anti-TL1A antibody comprises a HCDR1 having the sequence shown in SEQ ID NO: 68, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8. In some embodiments, the anti-TL1A antibody is afimkibart.

As a general principle, the treatment of IBD with anti-TL1A antibodies is described in PCT Publication No. WO 2021/260577 and US Patent Application Publication No. 2023/0235070, which are incorporated herein by reference for all purposes.

In some aspects of any of the methods of treatment provided herein, the IBD is ulcerative colitis (e.g., moderate to severe ulcerative colitis).

In some aspects of any of the methods of treatment provided herein, the patient has been determined to have a relatively low risk of a patient being non-responsive to a therapeutic dose of an anti-TL1A antibody. In some aspects, the patient has been determined to be a haplotype B non-carrier for TNFSF15. Methods for determining a patient's risk of non-responsiveness and for determining whether a patient is a haplotype B carrier for TNFSF15 are provided, e.g., in Section I herein.

### A. Induction Phase

Any of the methods provided herein may comprise administration of an anti-TL1A antibody in an induction phase. The induction phase may comprise administration of one or more doses of the anti-TL1A antibody.

In some aspects in which the induction phase comprises administration of at least two doses of the anti-TL1A antibody, the time interval between each individual induction dose may be the same. The individual induction dosages can be administered at least daily, at least one day apart, at least 1 week apart, at least 2 weeks apart, at least 3 weeks apart, at least 4 weeks apart, at least 5 weeks apart, at least 6 weeks apart, at least 7 weeks apart, at least 8 weeks apart, at least 9 weeks apart, at least 10 weeks apart, at least 11 weeks apart, or at least 12 weeks apart. In one preferred aspect, the individual induction dosages are administered one month apart. In one preferred aspect, the individual induction dosages are administered four weeks apart (Q4W).

In some aspects, the induction phase comprises administration of four doses of the anti-TL1A antibody. In some aspects, the doses of the anti-TL1A antibody are administered about 4 weeks apart (e.g., are administered 4 weeks apart). In some aspects, the induction phase has a duration of about 14 weeks, and (a) the first dose of the anti-TL1A antibody is administered on Day 1 of Week 0; (b) the second dose of the anti-TL1A antibody is administered on Day 1 of Week 4; (c) the third dose of the anti-TL1A antibody is administered on Day 1 of Week 8; and (d) the fourth dose of the anti-TL1A antibody is administered on Day 1 of Week 12.

The individual induction dose can be 1, 5, 10, 25, 50, 100, 150, 200, 250, 300, 350, 400, 450, 500, 550, 600, 650, 700, 750, 800, 850, 900, 950 or 1000 mg of the anti-TL1A antibody. In one preferred aspect, the individual induction dose is 50, 150, or 450 mg of the anti-TL1A antibody. In one aspect, the individual induction dose is 450 mg of the anti-TL1A antibody (e.g., each individual induction dose is administered at a dose of 450 mg). In another aspect, the individual induction dose is 150 mg of the anti-TL1A antibody (e.g., each individual induction dose is administered at a dose of 150 mg). In another aspect, the individual induction dose is 50 mg of the anti-TL1A antibody (e.g., each individual induction dose is administered at a dose of 50 mg). The individual induction dosages can be administered by any means. Preferably, the individual induction dosages are administered subcutaneously. In an aspect, the individual induction dose is 50, 150, or 450 mg administered subcutaneously. In an aspect, the individual induction dose is 50, 150, or 450 mg administered subcutaneously on a monthly basis. In an aspect, the individual induction dose is 50, 150, or 450 mg administered subcutaneously every four weeks (Q4W).

In some aspects, in the induction phase, the anti-TL1A antibody is administered subcutaneously (SC) at a dose of about 450 mg (e.g., a dose of 450 mg) (e.g., each dose in the induction phase comprises SC administration of about 450 mg (e.g., 450 mg) of the anti-TL1A antibody).

### B. Maintenance Phase

Any of the methods provided herein may comprise subcutaneous administration of an anti-TL1A antibody in a maintenance phase that follows administration of the anti-TL1A antibody in an induction phase. The maintenance phase may comprise administration of one or more doses of the anti-TL1A antibody.

The maintenance phase may be initiated at any appropriate time following the induction phase. In some aspects, the first dose of the maintenance phase is administered about two weeks (e.g., two weeks) after administration of the last dose of the induction phase. For example, in aspects in which the induction phase comprises administration of four doses of the anti-TL1A antibody, the first dose of the maintenance phase may be administered about two weeks (e.g., two weeks) after administration of the final dose of the induction phase.

In some aspects in which the maintenance phase comprises administration of at least two doses of the anti-TL1A antibody, the time interval between each individual maintenance dose may be the same. The individual maintenance doses can be administered at least daily, at least one day apart, at least 1 week apart, at least 2 weeks apart, at least 3 weeks apart, at least 1 month apart, at least 2 months apart, at least 3 months apart, at least 4 months apart, at least 5 months apart, or at least 6 months apart. In one preferred aspect, the individual maintenance doses are administered one month apart. In one preferred aspect, the individual maintenance doses are administered four weeks apart.

In some aspects, the maintenance phase comprises administration of the anti-TL1A antibody every four weeks (Q4W).

The individual maintenance dose can be 1, 5, 10, 25, 50, 100, 150, 200, 250, 300, 350, 400, 450, 500, 550, 600, 650, 700, 750, 800, 850, 900, 950 or 1000 mg. In a preferred aspect, the individual maintenance dose is 50, 150, or 450 mg. In one aspect, the individual maintenance dose is about 450 mg (e.g., is 450 mg). The individual maintenance dosages can be administered by any means.

Preferably, the individual maintenance dosages are administered subcutaneously. In an aspect, the individual maintenance dose is 50, 150, or 450 mg administered subcutaneously. In one aspect, the individual maintenance dose is 450 mg of the anti-TL1A antibody (e.g., each individual maintenance dose is administered subcutaneously at a dose of 450 mg). In another aspect, the individual maintenance dose is 150 mg of the anti-TL1A antibody (e.g., each individual maintenance dose is administered subcutaneously at a dose of 150 mg). In another aspect, the individual maintenance dose is 50 mg of the anti-TL1A antibody (e.g., each individual maintenance dose is administered subcutaneously at a dose of 50 mg). In an aspect, the individual maintenance dose is 50, 150, or 450 mg administered subcutaneously on a monthly basis.

In some aspects, the maintenance phase comprises administration of at least 10 doses of the anti-TL1A antibody (e.g., comprises 10, 11, 12, 13, 14, 15, 16, 17, 18, 19, 20, or more than 20 doses of the anti-TL1A antibody administered in a Q4W dosing regimen). In some aspects, the maintenance phase comprises administration of 10 doses of the anti-TL1A antibody (e.g., comprises ten doses of the anti-TL1A antibody administered in a Q4W dosing regimen).

Accordingly, in some aspects, the disclosure provides a method of treating an IBD in a patient, the method comprising administering to the patient an effective amount of an anti-TL1A antibody in a dosing regimen comprising an induction dosing regimen and a subsequent maintenance dosing regimen; wherein (a) the induction dosing regimen comprises subcutaneous administration of four individual induction doses of the anti-TL1A antibody at a dose of about 50 mg, 150 mg, or 450 mg (e.g., a dose of 50 mg, 150 mg, or 450 mg), wherein the individual induction doses are administered 4 weeks apart; and (b) the maintenance dosing regimen comprises subcutaneous administration of a plurality of individual maintenance doses of the anti-TL1A antibody at a dose of about 50 mg, 150 mg, or 450 mg (e.g., a dose of 50 mg, 150 mg, or 450 mg), wherein the individual maintenance doses are administered 4 weeks apart; and wherein the anti-TL1A antibody comprises: a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8. In some aspects, the anti-TL1A antibody is afimkibart.

In some aspects, the disclosure provides a method of treating IBD in a patient, the method comprising administering to the patient an effective amount of an anti-TL1A antibody in a dosing regimen comprising an induction dosing regimen and a subsequent maintenance dosing regimen; wherein (a) the induction dosing regimen comprises subcutaneous administration of four individual induction doses of the anti-TL1A antibody at a dose of about 450 mg (e.g., a dose of 450 mg), wherein the individual induction doses are administered 4 weeks apart; and (b) the maintenance dosing regimen comprises subcutaneous administration of a plurality of individual maintenance doses of the anti-TL1A antibody at a dose of about 450 mg (e.g., a dose of 450 mg), wherein the individual maintenance doses are administered 4 weeks apart; and wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In some aspects, the disclosure provides a method of treating an IBD in a patient, the method comprising administering to the patient an effective amount of an anti-TL1A antibody in a dosing regimen comprising an induction dosing regimen and a subsequent maintenance dosing regimen; wherein (a) the induction dosing regimen comprises subcutaneous administration of four individual induction doses of the anti-TL1A antibody at a dose of about 50 mg, 150 mg, or 450 mg (e.g., a dose of 50 mg, 150 mg, or 450 mg), wherein the individual induction doses are administered 4 weeks apart; and (b) the maintenance dosing regimen comprises subcutaneous administration of a plurality of individual maintenance doses of the anti-TL1A antibody at a dose of about 50 mg, 150 mg, or 450 mg (e.g., a dose of 50 mg, 150 mg, or 450 mg), wherein the individual maintenance doses are administered 4 weeks apart; and wherein the anti-TL1A antibody comprises: a HCDR1 having the sequence shown in SEQ ID NO: 68, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8. In some aspects, the anti-TL1A antibody is afimkibart.

In some aspects, the disclosure provides a method of treating IBD in a patient, the method comprising administering to the patient an effective amount of an anti-TL1A antibody in a dosing regimen comprising an induction dosing regimen and a subsequent maintenance dosing regimen; wherein (a) the induction dosing regimen comprises subcutaneous administration of four individual induction doses of the anti-TL1A antibody at a dose of about 450 mg (e.g., a dose of 450 mg), wherein the individual induction doses are administered 4 weeks apart; and (b) the maintenance dosing regimen comprises subcutaneous administration of a plurality of individual maintenance doses of the anti-TL1A antibody at a dose of about 450 mg (e.g., a dose of 450 mg), wherein the individual maintenance doses are administered 4 weeks apart; and wherein the anti-TL1A antibody comprises a HCDR1 having the sequence shown in SEQ ID NO: 68, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

In some aspects, the dosing regimen comprising the induction phase and maintenance phase has a duration of about 52 weeks (e.g., has a duration of 52 weeks).

### C. Response to Treatment

### Clinical Response

Following the induction dosing regimen and/or the maintenance dosing regimen, the patient may experience an improvement in signs and symptoms of IBD characterized by a clinical response. The term “clinical response” may be defined as a decrease from baseline of at least 3 points in total Mayo score with at least 30% change, accompanied by at least one-point decrease or absolute score of 0 or 1 in rectal bleeding subscore.

The abbreviation “Mayo” means the Mayo Scoring System for Assessment of Ulcerative Colitis Activity. “Adaptive Mayo Score” refers to the Adaptive Mayo Score system which has 3 subscores of the Mayo Score ranging from 0 to 9, without physician's global assessment (PGA) subscore.

In some aspects, in a population of patients treated according to any one of the methods provided herein, the treating results in an increase in the proportion of patients who have achieved a clinical response at the end of the induction phase as compared to a reference population. For example, in some aspects, the induction phase has a duration of about 14 weeks, and the treating results in an increase in the proportion of patients who have achieved a clinical response at Week 14. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved a clinical response at Week 14. In some aspects, the proportion of patients in the population of patients who have achieved a clinical response at Week 14 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved a clinical response at Week 14 in the reference population.

In some aspects, in a population of patients treated according to any one of the methods comprising a maintenance phase provided herein, the treating results in an increase in the proportion of patients who have achieved a clinical response at the end of the maintenance phase as compared to a reference population. For example, in some aspects, the dosing regimen has a duration of about 56 weeks, and the treating results in an increase in the proportion of patients who have achieved a clinical response at Week 56. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved a clinical response at Week 56. In some aspects, the proportion of patients in the population of patients who have achieved a clinical response at Week 56 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved a clinical response at Week 56 in the reference population.

In any of the methods provided herein, the reference population may be any population that serves as an appropriate control. For example, in some aspects, the reference population is a population of subjects having an IBD (e.g., UC, e.g., moderately or severely active UC) who have not been treated with an anti-TL1A antibody (e.g., have been treated with a placebo).

### Endoscopic Response

Following the induction dosing regimen and/or the maintenance dosing regimen, the patient may experience an improvement in signs and symptoms of IBD characterized by an endoscopic response. The term “endoscopic response” refers to a Mayo endoscopy subscore 0 or 1.

### Clinical Remission

Following the induction dosing regimen and/or the maintenance dosing regimen, the patient may experience an improvement in signs and symptoms of IBD characterized by a clinical remission.

In some aspects, in a population of patients treated according to any one of the methods provided herein, the treating results in an increase in the proportion of patients who have achieved clinical remission at the end of the induction phase as compared to a reference population. For example, in some aspects, the induction phase has a duration of about 14 weeks, and the treating results in an increase in the proportion of patients who have achieved clinical remission at Week 14. In some aspects, at least 1%, 5%, 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved clinical remission at Week 14. In some aspects, at least about 15% of patients in the population of patients have achieved clinical remission at Week 14. In some aspects, at least about 40% of patients in the population of patients have achieved clinical remission at Week 14. In some aspects, between 15% and 45.2% of patients in the population of patients have achieved clinical remission at Week 14. In some aspects, 23.3%, 23.9%, 25.5%, 29.8%, 31.8%, or 35% of patients in the population of patients have achieved clinical remission at Week 14.

In some aspects, the proportion of patients in the population of patients who have achieved clinical remission at Week 14 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved clinical remission at Week 14 in the reference population.

In some aspects, in a population of patients treated according to any one of the methods comprising a maintenance phase provided herein, the treating results in an increase in the proportion of patients who have achieved clinical remission at the end of the maintenance phase as compared to a reference population. For example, in some aspects, the dosing regimen has a duration of about 56 weeks, and the treating results in an increase in the proportion of patients who have achieved clinical remission at Week 56. In some aspects, at least 1%, 5%, 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved clinical remission at Week 56. In some aspects, at least about 20% of patients in the population of patients have achieved clinical remission at Week 56. In some aspects, at least about 55% of patients in the population of patients have achieved clinical remission at Week 56. In some aspects, between 19.4% and 56.5% of patients in the population of patients have achieved clinical remission at Week 56. In some aspects, 31.0%, 34.6%, 35.7%, 38.5%, or 39.3% of patients in the population of patients have achieved clinical remission at Week 56.

In some aspects, the proportion of patients in the population of patients who have achieved clinical remission at Week 56 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved clinical remission at Week 56 in the reference population.

In some aspects, in a population of patients treated according to any one of the methods comprising a maintenance phase provided herein, the treating results in an increase in the proportion of patients who experience maintenance of remission throughout the maintenance phase as compared to a reference population. For example, in some aspects, the induction phase has a duration of about 14 weeks, the entire dosing regimen has a duration of about 56 weeks, and the treating results in an increase in the proportion of patients who are experiencing clinical remission at Week 14 and at Week 56.

In some aspects, clinical remission is defined as a modified Mayo Score (mMS)≤2 with stool frequency subscore (SFS)=0 or 1, rectal bleeding subscore (RBS)=0, and endoscopic subscore (ES)=0 or 1.

In other aspects, the term “clinical remission” is based on 12-point total Mayo score: total Mayo score ≤2 with no individual subscore >1. In some aspects, Per Adapted (Modified) Mayo score is defined as endoscopic subscore=0 or 1, >=1-point decrease from baseline to achieve a stool frequency subscore=0 or 1, and rectal bleeding subscore=0. In some aspects, Per Adapted (Modified) Mayo score is defined as endoscopic subscore=0 or 1, a stool frequency subscore=0 or 1 with no increase from baseline, and rectal bleeding subscore=0.

### Endoscopic Remission

Following the induction dosing regimen and/or the maintenance dosing regimen, the patient may experience an improvement in signs and symptoms of IBD characterized by an endoscopic remission. The term “endoscopic remission” refers to a Mayo endoscopy subscore of 0. In some aspects, the patient experiences endoscopic remission at or before the end of the induction phase, e.g., at or before Week 14 of treatment. In some aspects, in a population of patients treated according to any one of the methods provided herein, the treating results in an increase in the proportion of patients who have achieved endoscopic remission at the end of the induction phase as compared to a reference population. In some aspects, the patient experiences endoscopic remission at or before the end of the maintenance phase, e.g., at or before Week 56 of treatment. In some aspects, in a population of patients treated according to any one of the methods provided herein, the treating results in an increase in the proportion of patients who have achieved endoscopic remission at the end of the maintenance phase as compared to a reference population.

### Deep Remission

Following the induction dosing regimen and/or the maintenance dosing regimen, the patient may experience an improvement in signs and symptoms of IBD characterized by a deep remission. The term “deep remission” refers to a total Mayo score of 2 points or lower, with no individual subscore exceeding 1 point and a 0 on both endoscopic and rectal bleeding subscore.

In some aspects, in a population of patients treated according to any one of the methods provided herein, the treating results in an increase in the proportion of patients who have achieved deep remission at the end of the induction phase as compared to a reference population. In some aspects, the patient experiences deep remission at or before the end of the maintenance phase, e.g., at or before Week 56 of treatment. In some aspects, in a population of patients treated according to any one of the methods provided herein, the treating results in an increase in the proportion of patients who have achieved deep remission at the end of the maintenance phase as compared to a reference population.

### Symptomatic Remission

Following the induction dosing regimen and/or the maintenance dosing regimen, the patient may experience an improvement in signs and symptoms of IBD characterized by a symptomatic remission. The term “symptomatic remission” refers to a total Mayo score of 2 points or lower, with no individual subscore exceeding 1 point, and both rectal bleeding and stool frequency subscores of 0.

In some aspects, in a population of patients treated according to any one of the methods provided herein, the treating results in an increase in the proportion of patients who have achieved symptomatic remission at the end of the induction phase as compared to a reference population. In some aspects, the patient experiences symptomatic remission at or before the end of the maintenance phase, e.g., at or before Week 56 of treatment. In some aspects, in a population of patients treated according to any one of the methods provided herein, the treating results in an increase in the proportion of patients who have achieved symptomatic remission at the end of the maintenance phase as compared to a reference population.

### Endoscopic Improvement

Following the induction dosing regimen and/or the maintenance dosing regimen, the patient may experience an improvement in signs and symptoms of IBD characterized by an endoscopic improvement. The term “endoscopic improvement” (“EI”) refers to a decrease of ≥1 point in Mayo endoscopy subscore or an absolute endoscopy score of ≤1. In some aspects, endoscopic improvement is defined as an endoscopic subscore=0 or 1.

In some aspects, the patient experiences endoscopic improvement at or before the end of the induction phase, e.g., at or before Week 14 of treatment. In some aspects, in a population of patients treated according to any one of the methods provided herein, the treating results in an increase in the proportion of patients who have achieved endoscopic improvement at the end of the induction phase as compared to a reference population. For example, in some aspects, the induction phase has a duration of about 14 weeks, and the treating results in an increase in the proportion of patients who have achieved endoscopic improvement at Week 14. In some aspects, at least 1%, 5%, 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved endoscopic improvement at Week 14. In some aspects, at least about 25% of patients in the population of patients have achieved endoscopic improvement at Week 14. In some aspects, at least about 50% of patients in the population of patients have achieved endoscopic improvement at Week 14. In some aspects, between 9.6% and 53.5% of patients in the population of patients have achieved endoscopic improvement at Week 14. In some aspects, 38.3%, 40.4%, or 40.9% of patients in the population of patients have achieved endoscopic improvement at Week 14.

In some aspects, the proportion of patients in the population of patients who have achieved endoscopic improvement at Week 14 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved endoscopic improvement at Week 14 in the reference population.

In some aspects, the patient experiences endoscopic improvement at or before the end of the maintenance phase, e.g., at or before Week 56 of treatment. In some aspects, in a population of patients treated according to any one of the methods provided herein, the treating results in an increase in the proportion of patients who have achieved endoscopic improvement at the end of the maintenance phase as compared to a reference population. In some aspects, at least 1%, 5%, 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved endoscopic improvement at Week 56. In some aspects, at least 20% of patients in the population of patients have achieved endoscopic improvement at Week 56. In some aspects, at least 65% of patients in the population of patients have achieved endoscopic improvement at Week 56. In some aspects, between 23.8% and 66.7% of patients in the population of patients have achieved endoscopic improvement at Week 56. In some aspects, 38.1%, 39.3%, or 50.0% of patients in the population of patients have achieved endoscopic improvement at Week 56.

In some aspects, the proportion of patients in the population of patients who have achieved endoscopic improvement at Week 56 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved endoscopic improvement at Week 56 in the reference population.

### Improvement in Signs and Symptoms of IBD

Following the induction dosing regimen and/or the maintenance dosing regimen, the patient may experience an improvement in signs and symptoms of IBD that are maintained while the patient receives the maintenance dosing regimen.

In some aspects of the disclosure, the induction dosing regimen and/or the maintenance dosing regimen, with the anti-TL1A antibody effectively improves signs and symptoms of IBD by at least 14 weeks after starting of treatment with the anti-TL1A antibody. These improvement in signs and symptoms of IBD may be characterized by an improvement in the Mayo endoscopic subscore. The reduction of the patient's Mayo endoscopic subscore may be by at least 1, 2, or 3 or more integers.

The improvement in signs and symptoms of IBD may be characterized by the patient having a Mayo endoscopic subscore of 0 or 1, 2, or 3. The improvement in signs and symptoms of IBD may be characterized by the patient having a total Mayo score of 0, 1, 2, or 3. The improvement in signs and symptoms of IBD may be characterized by the patient having a Robarts Histopathology Index (RHI) of less than 5. The improvement in signs and symptoms of IBD may be characterized by the patient having a Geboes Index of less than 3.2.

The improvement in signs and symptoms of IBD may be maintained during the maintenance dosing regimen for at least 2, 3, 4, 6, or 12 months.

### D. Prior Treatment

In some aspects, the patient was previously treated with corticosteroids prior to administering the anti-TL1A antibody. In some aspects the patient was previously treated with one or more treatments selected from the group consisting of tumor necrosis factor inhibitors, anti-integrins, azathioprine, 6-mercaptopurine, and methotrexate.

In some aspects, the patient has previously been treated with a therapy for IBD (e.g., a therapy for UC) and has experienced inadequate response to the therapy, loss of response to the therapy, and/or intolerance of the therapy. In some aspects, the therapy was a conventional therapy for UC, e.g., comprised administration of a steroid or an immunosuppressant (e.g., the patient has experienced inadequate response to, loss of response to, and/or intolerance of a steroid or an immunosuppressant).

In some aspects, the therapy was an advanced therapy for UC, e.g., comprised administration of an anti-tumor necrosis factor (TNF) agent, an anti-integrin agent, an anti-IL12/IL23 agent, and/or a Janus kinase (JAK) inhibitor (e.g., the patient has experienced inadequate response to, loss of response to, and/or intolerance of an anti-TNF agent, an anti-integrin agent, an anti-IL12/IL23 agent, and/or a JAK inhibitor). Thus, in some aspects, the patient had received prior advanced therapy at baseline (e.g., before administration of the first dose of the induction phase). In other aspects, the patient had not received prior advanced therapy at baseline.

E. Fecal Calprotectin and hsCRP

In some aspects the patient shows a reduction of fecal calprotectin from baseline of at least 50% during the course of treatment (e.g., from week 2 to week 26 of treatment). In some aspects the patient shows a reduction of fecal calprotectin from baseline of at least 60% from week 2 to week 26 treatment. In some aspects the patient shows a reduction of high-sensitivity C-reactive protein (hsCRP) from baseline from week 2 to week 26 of treatment.

### F. Inflammatory Bowel Diseases

In some aspects the IBD is ulcerative colitis (UC). In some aspects, the patient has moderate to severe ulcerative colitis. The term “moderate to severe ulcerative colitis” is defined as having an Adapted Mayo score of 5 to 9, with an endoscopy subscore of 2 or 3 and/or as a total Mayo score of at least 6 and endoscopic subscore of at least 2.

In some aspects, the IBD is Crohn's disease (CD). In some aspects, the patient has moderate to severe CD. The term “moderate to severe CD” is defined by Crohn's Disease Activity Index (CDAI) and Simple Endoscopic Score for CD (SES-CD).

In some aspects, the patient has a diagnosis of ulcerative colitis, indeterminate colitis, microscopic colitis, ischemic colitis, infectious colitis, radiation colitis or active diverticular disease.

### III. Kits

The disclosure also provides kits comprising any or all of the anti-TL1A antibodies described herein. Kits of the disclosure include one or more containers comprising an anti-TL1A antibody described herein and instructions for use in accordance with any of the methods of the disclosure described herein. Generally, these instructions comprise a description of administration of the anti-TL1A antibody for the above described therapeutic treatments. In some aspects, kits are provided for producing a single-dose administration unit. In certain aspects, the kit can contain both a first container having a dried protein and a second container having an aqueous formulation. In certain aspects, kits containing single and multi-chambered pre-filled syringes (e.g., liquid syringes and lyosyringes) are included.

The instructions relating to the use of an anti-TL1A antibody generally include information as to dosage, dosing schedule, and route of administration for the intended treatment. The containers may be unit doses, bulk packages (e.g., multi dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-TL1A antibody. The container may further comprise a second pharmaceutically active agent.

Kits may optionally provide additional components such as buffers and interpretive information. Normally, the kit comprises a container and a label or package insert(s) on or associated with the container.

### IV. Anti-TL1A and Anti-DR3 Antibodies

The methods of the present disclosure include the administration of an anti-TL1A antibody. Exemplary anti-TL1A antibodies of the disclosure are set forth in Table 2 and below. Exemplary anti-DR3 antibodies of the disclosure are also provided below.

TABLE 2
Sequences of Exemplary Antibodies of the Disclosure

| SEQ ID | | |
|---|---|---|
| number | Description | Amino Acid Sequence |
| 1 | Afimkibart VH | QVQLVQSGAEVKKPGASVKVSCKASGYDFTYYGISWVRQAPG |
| | | QGLEWMGWISTYNGNTHYARMLQGRVTMTTDTSTRTAYMELR |
| | | SLRSDDTAVYYCARENYYGSGAYRGGMDVWGQGTTVTVSS |
| 2 | Afimkibart VL | EIVLTQSPATLSLSPGERATLSCRASQSVSSYLAWYQQKPGQAP |
| | | RLLIYDASNRATGIPARFSGSGSGTDFTLTISSLEPEDFAVYYCQ |
| | | QRSNWPWTFGQGTKVEIK |
| 3 | Afimkibart | YYGIS |
| | HCDR1 | |
| | (CDR-H1) | |
| 4 | Afimkibart | WISTYNGNTHYARMLQG |
| | HCDR2 | |
| | (CDR-H2) | |
| 5 | Afimkibart | ENYYGSGAYRGGMDV |
| | HCDR3 | |
| | (CDR-H3) | |
| 6 | Afimkibart | RASQSVSSYLA |
| | LCDR1 | |
| | (CDR-L1) | |
| 7 | Afimkibart | DASNRAT |
| | LCDR2 | |
| | (CDR-L2) | |
| 8 | Afimkibart | QQRSNWPWT |
| | LCDR3 | |
| | (CDR-L3) | |
| 9 | Afimkibart HC | QVQLVQSGAEVKKPGASVKVSCKASGYDFTYYGISWVRQAPG |
| | | QGLEWMGWISTYNGNTHYARMLQGRVTMTTDTSTRTAYMELR |
| | | SLRSDDTAVYYCARENYYGSGAYRGGMDVWGQGTTVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCDKTHTCPPCPAPEAAGAPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNV |
| | | FSCSVMHEALHNHYTQKSLSLSPG |
| 10 | Afimkibart LC | EIVLTQSPATLSLSPGERATLSCRASQSVSSYLAWYQQKPGQAP |
| | | RLLIYDASNRATGIPARFSGSGSGTDFTLTISSLEPEDFAVYYCQ |
| | | QRSNWPWTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 11 | 1D1-1.27_VH | QVQLVQSGAEVKKPGASVKVSCKASGYDFTYYGISWVRQAPG |
| | | QGLEWMGWISTYNGNTHYARMLQGRVTMTTDTSTRTAYMELR |
| | | SLRSDDTAVYYCARENYYGSGSYRGGMDVWGQGTTVTVSS |
| 12 | 1D1-1.28_VH | QVQLVQSGAEVKKPGASVKVSCKASGYDFTYYGISWVRQAPG |
| | | QGLEWMGWISTYNGNKHYARMLQGRVTMTTDTSTRTAYMELR |
| | | SLRSDDTAVYYCARENYYGSGSYRGGMDVWGQGTTVTVSS |
| 13 | 1D1-1.29_VH | QVQLVQSGAEVKKPGASVKVSCKASGYDFTYYGISWVRQAPG |
| | | QGLEWMGWISTYNGGTHYARMLQGRVTMTTDTSTRTAYMELR |
| | | SLRSDDTAVYYCARENYYGSGSYRGGMDVWGQGTTVTVSS |
| 14 | 1D1-1.30_VH | QVQLVQSGAEVKKPGASVKVSCKASGYDFTYYGISWVRQAPG |
| | | QGLEWMGWISTYNGVTHYARMLQGRVTMTTDTSTRTAYMELR |
| | | SLRSDDTAVYYCARENYYGSGSYRGGMDVWGQGTTVTVSS |
| 15 | 1D1-1.32_VH | QVQLVQSGAEVKKPGASVKVSCKASGYDFTYYGISWVRQAPG |
| | | QGLEWMGWISTYNGGTHYARMLQGRVTMTTDTSTRTAYMELR |
| | | SLRSDDTAVYYCARENYYGSGAYRGGMDAWGQGTTVTVSS |
| 16 | 1D1-1.33_VH | QVQLVQSGAEVKKPGASVKVSCKASGYDFTYYGISWVRQAPG |
| | | QGLEWMGWISTYNGVTHYARMLQGRVTMTTDTSTRTAYMELR |
| | | SLRSDDTAVYYCARENYYGSGAYRGGMDAWGQGTTVTVSS |
| 17 | 1D1-1.34_VH | QVQLVQSGAEVKKPGASVKVSCKASGYDFTYYGISWVRQAPG |
| | | QGLEWMGWISTYNGKTHYARMHQGRVTMTTDTSTRTAYMELR |
| | | SLRSDDTAVYYCARENYYGSGAYRGGMDAWGQGTTVTVSS |
| 18 | 15A9_VH | QVQLVQSGAEVKKPGASLKVSCKASGYPFTNYGISWVRQAPGQ |
| | | GLEWMGWISTYNGNTHYAQKLQGRVTMTTDTSTTTAYMDLRSL |
| | | RSDDTAVYYCARENYYGSGSYRGGMDVWGQGTTVTVSS |
| 19 | 15C11_VH | QVQLVQSGAEVKKPGASVKVSCKASGYSFTTYGISWVRQAPGQ |
| | | GLEWMGWISTYNGNTHYAQKLQGRVTMTTDTSTRTAYMELRSL |
| | | RSDDTAVYYCARENYYGSGSYRGGMDVWGQGTTVTVSS |
| 20 | 7D4_VH | QVQLVQSGAEVKKPGASVKVSCKASGYTFTSYGINWVRQAPG |
| | | QGLEWMGWISTYNGNTNSAQKLQGRVTMTTDTSTSTAYMELR |
| | | SLRSDDTAVYYCARAHSSSWFDAFDIWGQGTMVTVSS |
| 21 | 26B11_VH | QVQLVESGGGVVQPGRSLRLSCAASGFTFSSFAMHWVRQAPG |
| | | KGLEWVALIPFDGSSNYYADSVKGRFTISRDNSKNTLYLQMNSL |
| | | RAEDTAVYYCARDRNYYGSGSFSFDAFDIWGQGTLVTVSS |
| 22 | 9B3_VH | QVQLVESGGGVVQPGRSLRLSCAASGFTFSNYALHWVRQAPG |
| | | KGLEWVALISYDGSDKYYADSVKGRFAISRDNSKNTLYLQMNSL |
| | | RAEDTAVYYCARDREYCTYSSCSYDAFDIWGQGTMVTVSS |
| 23 | 22F9_VH | QVQLVQSGAEVKKPGASVKVSCKASGYTFTSYAMHWVRQAPG |
| | | QRLEWMGWINAGNGNTKYSQKFQGRVTITRDTSASTAYMELSS |
| | | LRSEDTAVYYCARGYSSAWFDAFDIWGQGTMVTVSS |
| 24 | 7D4_VL | AIQLTQSPSSLSASVGDRVTITCRASQGISSALAWYQQKPGKAP |
| | | KLLIYDASSLESGVPSRFSGSGSGTDFTLTISSLQPEDFATYYCQ |
| | | QFNSYPLTFGGGTKVEIK |
| 25 | 26B11_VL | DIQMTQSPSSLSASVGDRVTITCRASQGISNWLAWYQQKPEKA |
| | | PKSLIYAASSLQSGVPSRFSGSGSGTDFTLTISSLQPEDFATYYC |
| | | QQYNSYPYTFGQGTKLEIK |
| 26 | 9B3_VL | DIQMTQSPSSLSASVGDRVTITCRASQGISSWLAWYQQKPEKA |
| | | PKSLIYAASSLQSGVPSRFSGSGSGTDFTLTISSLQPEDYATYYC |
| | | QQYNSYPYTFGQGTKLEIK |
| 27 | 22F9_VL | AIQLTQSPSSLSASVGDRVTITCRASQGISSALAWYQQKPGKAP |
| | | KLLIYDASSLESGVPSRFSGSGSGTDFTLTISSLQPEDFATYYCQ |
| | | QFNSYPLTFGGGTKVEIK |
| 28 | SEQ ID NO: 18 | EVQLLESGGGLVQPGKSLRLSCAVSGFTFSTYGMNWVRQAPG |
| | of WO | KGLEWVSSISGTGRTTYHADSVQGRFTVSRDNSKNILYLQMNSL |
| | 2012/064682 | RADDTAVYFCTKERGDYYYGVFDYWGQGTLVTVSS |
| 29 | SEQ ID NO: 26 | DIQMTQSPSTLSASVGDRVTITCRASQTISSWLAWYQQTPEKAP |
| | of WO | KLLIYAASNLQSGVPSRFSGSGSGTEFTLTISSLQPDDFATYYCQ |
| | 2012/064682 | QYHRSWTFGQGTKVEIT |
| 30 | C320-168 | QVQLVQSGAEVKKPGASVKVSCKASGYTFTSYDINWVRQAPGQ |
| | huIgG1-EFN of | GLEWMGWLNPNSGNTGYAQKFQGRVTMTRNTSISTAYMELSS |
| | WO | LRSEDTAVYYCAREVPETAAFEYWGQGTLVTVSSASTKGPSVF |
| | 2013/044298 | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPEAAGAPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 31 | C320-168 | QSVLTQPPSVSGAPGQRVTISCTSSSSDIGAGLGVHWYQQLPG |
| | huLambda of | TAPKLLIYGYYNRPSGVPDRFSGSKSGTSASLTITGLLPEDEGDY |
| | WO | YCQSYDGTLSALFGGGTKLTVLGQPKAAPSVTLFPPSSEELQAN |
| | 2013/044298 | KATLVCLISDFYPGAVTVAWKADSSPVKAGVETTTPSKQSNNKY |
| | | AASSYLSLTPEQWKSHRSYSCQVTHEGSTVEKTVAPTECS |
| 32 | VH C320-179 | QVQLVQSGAEVKKPGASVKVSCKASGYTFTSYDINWVRQAPGQ |
| | of WO | GLEWMGWLNPNSGNTGYAQKFQGRVTMTADRSTSTAYMELSS |
| | 2013/044298 | LRSEDTAVYYCAREVPETAAFEYWGQGTLVTVSS |
| 33 | VL C320-179 of | QSVLTQPPSVSGAPGQRVTISCTSSSSDIGAGLGVHWYQQLPG |
| | WO | TAPKLLIEGYYNRPSGVPDRFSGSKSGTSASLTITGLLPEDEGDY |
| | 2013/044298 | YCQSYDGTLSALFGGGTKLTVLG |
| 34 | Afimkibart HC | QVQLVQSGAEVKKPGASVKVSCKASGYDFTYYGISWVRQAPG |
| | (with C-terminal | QGLEWMGWISTYNGNTHYARMLQGRVTMTTDTSTRTAYMELR |
| | lysine) | SLRSDDTAVYYCARENYYGSGAYRGGMDVWGQGTTVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCDKTHTCPPCPAPEAAGAPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNV |
| | | FSCSVMHEALHNHYTQKSLSLSPGK |
| 35 | Tulisokibart HC | QVQLVQSGAEVKKPGASVKVSCKASGFDIQDTYMHWVKQRPG |
| | | QGLEWMGRIDPASGHTKYDPKFQVRVTITRDTSTSTVYLELSSL |
| | | RSEDTAVYYCARSGGLPDVWGQGTTVTVSSASTKGPSVFPLAP |
| | | SSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALAAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 36 | Tulisokibart | QVQLVQSGAEVKKPGASVKVSCKASGFDIQ |
| | HFR1 | |
| 37 | Tulisokibart | DTYMH |
| | CDR-H1 | |
| 38 | Tulisokibart | WVKQRPGQGLEWMG |
| | HFR2 | |
| 39 | Tulisokibart | RIDPASGHTKYDPKFQV |
| | CDR-H2 | |
| 40 | Tulisokibart | RVTITRDTSTSTVYLELSSLRSEDTAVYYCAR |
| | HFR3 | |
| 41 | Tulisokibart | SGGLPDV |
| | CDR-H3 | |
| 42 | Tulisokibart | WGQGTTVTVSS |
| | HFR4 | |
| 43 | Tulisokibart HC | ASTKGPSVFPLAPSSKSTSGGTAALGCLVKDYFPEPVTVSWNS |
| | Tail | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAKTK |
| | | PREEQYNSTYRVVSVLTVLHQDWLNGKEYKCKVSNKALAAPIEK |
| | | TISKAKGQPREPQVYTLPPSRDELTKNQVSLTCLVKGFYPSDIAV |
| | | EWESNGQPENNYKTTPPVLDSDGSFFLYSKLTVDKSRWQQGN |
| | | VFSCSVMHEALHNHYTQKSLSLSPGK |
| 44 | Tulisokibart LC | EIVLTQSPGTLSLSPGERATLSCRASSSVSYMYWYQQKPGQAP |
| | | RPLIYATSNLASGIPDRFSGSGSGTDFTLTISRLEPEDFAVYYCQ |
| | | QWEGNPRTFGGGTKLEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 45 | Tulisokibart | EIVLTQSPGTLSLSPGERATLSC |
| | LFR1 | |
| 46 | Tulisokibart | RASSSVSYMY |
| | CDR-L1 | |
| 47 | Tulisokibart | WYQQKPGQAPRPLIY |
| | LFR2 | |
| 48 | Tulisokibart | ATSNLAS |
| | CDR-L2 | |
| 49 | Tulisokibart | GIPDRFSGSGSGTDFTLTISRLEPEDFAVYYC |
| | LFR3 | |
| 50 | Tulisokibart | QQWEGNPRT |
| | CDR-L3 | |
| 51 | Tulisokibart | FGGGTKLEIKRTV |
| | LFR4 | |
| 52 | Tulisokibart LC | AAPSVFIFPPSDEQLKSGTASVVCLLNNFYPREAKVQWKVDNAL |
| | Tail | QSGNSQESVTEQDSKDSTYSLSSTLTLSKADYEKHKVYACEVT |
| | | HQGLSSPVTKSFNRGEC |
| 53 | Tulisokibart VH | QVQLVQSGAEVKKPGASVKVSCKASGFDIQDTYMHWVKQRPG |
| | | QGLEWMGRIDPASGHTKYDPKFQVRVTITRDTSTSTVYLELSSL |
| | | RSEDTAVYYCARSGGLPDVWGQGTTVTVSS |
| 54 | Tulisokibart VL | EIVLTQSPGTLSLSPGERATLSCRASSSVSYMYWYQQKPGQAP |
| | | RPLIYATSNLASGIPDRFSGSGSGTDFTLTISRLEPEDFAVYYCQ |
| | | QWEGNPRTFGGGTKLEIKRTV |
| 55 | SEQ ID NO: 15 | GYTFTSYDIN |
| | of U.S. Pat. | |
| | No. 10,138,296 | |
| | (CDR-H1 of | |
| | variant 320- | |
| | 587) | |
| 56 | SEQ ID NO: 21 | WLNPNSGYTG |
| | of U.S. Pat. | |
| | No. 10,138,296 | |
| | (CDR-H2 of | |
| | variant 320- | |
| | 587) | |
| 57 | SEQ ID NO: 17 | EVPETAAFEY |
| | of U.S. Pat. | |
| | No. 10,138,296 | |
| | (CDR-H3 of | |
| | variant 320- | |
| | 587) | |
| 58 | SEQ ID NO: 18 | TSSSSDIGAGLGVH |
| | of U.S. Pat. | |
| | No. 10,138,296 | |
| | (CDR-L1 of | |
| | variant 320- | |
| | 587) | |
| 59 | SEQ ID NO: 19 | GYYNRPS |
| | of U.S. Pat. | |
| | No. 10,138,296 | |
| | (CDR-L2 of | |
| | variant 320- | |
| | 587) | |
| 60 | SEQ ID NO: 22 | QSWDGTLSAL |
| | of U.S. Pat. | |
| | No. 10,138,296 | |
| | (CDR-L3 of | |
| | variant 320- | |
| | 587) | |
| 61 | SEQ ID NO: 3 | QVQLVQSGAEVKKPGASVKVSCKASGYTFTSYDINWVRQAPGQ |
| | of U.S. Pat. | GLEWMGWLNPNSGYTGYAQKFQGRVTMTADRSTSTAYMELSS |
| | No. 10,138,296 | LRSEDTAVYYCAREVPETAAFEYWGQGTLVTVSS |
| | (VH of variant | |
| | 320-587) | |
| 62 | SEQ ID NO: 4 | QSVLTQPPSVSGAPGQRVTISCTSSSSDIGAGLGVHWYQQLPG |
| | of U.S. Pat. | TAPKLLIEGYYNRPSGVPDRFSGSKSGTSASLTITGLLPEDEGDY |
| | No. 10,138,296 | YCQSWDGTLSALFGGGTKLTVLG |
| | (VL of variant | |
| | 320-587) | |
| 63 | SEQ ID NO: 60 | QVQLVQSGAEVKKPGASVKVSCKASGYTFTSYDINWVRQAPGQ |
| | of U.S. Pat. | GLEWMGWLNPNSGYTGYAQKFQGRVTMTADRSTSTAYMELSS |
| | No. 10,138,296 | LRSEDTAVYYCAREVPETAAFEYWGQGTLVTVSSASTKGPSVF |
| | (HC) | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPELLGGPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 64 | SEQ ID NO: 61 | QSVLTQPPSVSGAPGQRVTISCTSSSSDIGAGLGVHWYQQLPG |
| | of U.S. Pat. | TAPKLLIEGYYNRPSGVPDRFSGSKSGTSASLTITGLLPEDEGDY |
| | No. 10,138,296 | YCQSWDGTLSALFGGGTKLTVLGQPKAAPSVTLFPPSSEELQA |
| | (LC) | NKATLVCLISDFYPGAVTVAWKADSSPVKAGVETTTPSKQSNNK |
| | | YAASSYLSLTPEQWKSHRSYSCQVTHEGSTVEKTVAPTECS |
| 65 | SEQ ID NOS: | QVQLVQSGAEVKKPGASVKVSCKASGYTFTSYDINWVRQAPGQ |
| | 60 and 62 of | GLEWMGWLNPNSGYTGYAQKFQGRVTMTADRSTSTAYMELSS |
| | U.S. Pat. No. | LRSEDTAVYYCAREVPETAAFEYWGQGTLVTVSSASTKGPSVF |
| | 10,138,296 | PLAPSSKSTSGGTAALGCLVKDYFPEPVTVSWNSGALTSGVHT |
| | (HC with | FPAVLQSSGLYSLSSVVTVPSSSLGTQTYICNVNHKPSNTKVDK |
| | L234A/L235A/ | KVEPKSCDKTHTCPPCPAPEAAGAPSVFLFPPKPKDTLMISRTP |
| | G237A) | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 66 | SEQ ID NOS: | QVQLVQSGAEVKKPGASVKVSCKASGYTFTSYDINWVRQAPGQ |
| | 60 and 64 of | GLEWMGWLNPNSGYTGYAQKFQGRVTMTADRSTSTAYMELSS |
| | U.S. Pat. No. | LRSEDTAVYYCAREVPETAAFEYWGQGTLVTVSSASTKGPSVF |
| | 10,138,296 | PLAPSSKSTSGGTAALGCLVKDYFPEPVTVSWNSGALTSGVHT |
| | (HC with YTE) | FPAVLQSSGLYSLSSVVTVPSSSLGTQTYICNVNHKPSNTKVDK |
| | | KVEPKSCDKTHTCPPCPAPELLGGPSVFLFPPKPKDTLYITREPE |
| | | VTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 67 | SEQ ID NOS: | QVQLVQSGAEVKKPGASVKVSCKASGYTFTSYDINWVRQAPGQ |
| | 60 and 66 of | GLEWMGWLNPNSGYTGYAQKFQGRVTMTADRSTSTAYMELSS |
| | U.S. Pat. No. | LRSEDTAVYYCAREVPETAAFEYWGQGTLVTVSSASTKGPSVF |
| | 10,138,296 | PLAPSSKSTSGGTAALGCLVKDYFPEPVTVSWNSGALTSGVHT |
| | (HC with | FPAVLQSSGLYSLSSVVTVPSSSLGTQTYICNVNHKPSNTKVDK |
| | L235A/G237A) | KVEPKSCDKTHTCPPCPAPELAGAPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 68 | Afimkibart long | GYDFTYYGIS |
| | version of | |
| | HCDR1 (CDR- | |
| | H1) | |
| 69 | Afimkibart | QVQLVQSGAEVKKPGASVKVSCKASGYDFT |
| | heavy chain | |
| | framework | |
| | region 1 | |
| | (HFR1) | |
| 70 | Afimkibart | WVRQAPGQGLEWMG |
| | HFR2 | |
| 71 | Afimkibart | RVTMTTDTSTRTAYMELRSLRSDDTAVYYCAR |
| | HFR3 | |
| 72 | Afimkibart | WGQGTTVTVSS |
| | HFR4 | |
| 73 | Afimkibart light | EIVLTQSPATLSLSPGERATLSC |
| | chain | |
| | framework | |
| | region 1 (LFR1) | |
| 74 | Afimkibart | WYQQKPGQAPRLLIY |
| | LFR2 | |
| 75 | Afimkibart | GIPARFSGSGSGTDFTLTISSLEPEDFAVYYQ |
| | LFR3 | |
| 76 | Afimkibart | FGQGTKVEIKRTV |
| | LFR4 | |
| 77 | Afimkibart short | QVQLVQSGAEVKKPGASVKVSCKAS |
| | version of | |
| | HFR1 | |

In some aspects of the disclosure, the anti-TL1A antibody comprises three CDRs from the variable heavy chain region having the sequence shown in SEQ ID NO: 1 and three CDRs from the variable light chain region having the sequence shown in SEQ ID NO: 2.

In some aspects of the disclosure, the anti-TL1A antibody comprises a HCDR1 (CDR H1) having the sequence shown in SEQ ID NO: 3, a HCDR2 (CDR H2) having the sequence shown in SEQ ID NO: 4, a HCDR3 (CDR H3) having the sequence shown in SEQ ID NO: 5, a LCDR1 (CDR L1) having the sequence shown in SEQ ID NO: 6, a LCDR2 (CDR L2) having the sequence shown in SEQ ID NO: 7, and a LCDR3 (CDR L3) having the sequence shown in SEQ ID NO: 8. In some aspects, the anti-TL1A antibody comprises one, two, three, four, five, six, seven, or all eight of the framework region sequences shown in SEQ ID NOs: 69-76.

In some aspects of the disclosure, the anti-TL1A antibody comprises a HCDR1 (CDR H1) having the sequence shown in SEQ ID NO: 68, a HCDR2 (CDR H2) having the sequence shown in SEQ ID NO: 4, a HCDR3 (CDR H3) having the sequence shown in SEQ ID NO: 5, a LCDR1 (CDR L1) having the sequence shown in SEQ ID NO: 6, a LCDR2 (CDR L2) having the sequence shown in SEQ ID NO: 7, and a LCDR3 (CDR L3) having the sequence shown in SEQ ID NO: 8. In some aspects, the anti-TL1A antibody comprises one, two, three, four, five, six, seven, or all eight of the framework region sequences shown in SEQ ID NOs: 70-77.

In some aspects of the disclosure, the anti-TL1A antibody comprises a variable heavy chain region having the sequence shown in SEQ ID NO: 1 and a variable light chain region having the sequence shown in SEQ ID NO: 2.

In some aspects of the disclosure, the anti-TL1A antibody comprises a heavy chain having the sequence shown in SEQ ID NO: 34. In some embodiments, the anti-TL1A antibody comprises a heavy chain having the sequence shown in SEQ ID NO: 34 and a light chain having the sequence shown in SEQ ID NO: 10, wherein the C-terminal lysine (K) of the heavy chain amino acid sequence of SEQ ID NO: 34 is optional. In some embodiments, the heavy chain does not have the C-terminal lysine (K). In some embodiments, the heavy chain has the sequence shown in SEQ ID NO: 9. In some embodiments, the anti-TL1A antibody comprises a heavy chain having the sequence shown in SEQ ID NO: 9 and a light chain having the sequence shown in SEQ ID NO: 10.

In some aspects of the disclosure, the anti-TL1A antibody is afimkibart (also known as RO7790121, RVT-3101, or PF-06480605).

In some aspects of the disclosure, the anti-TL1A antibody comprises a VH encoded by the nucleic acid sequence of the insert of the vector deposited as 1 D1 1.31 VH having ATCC accession number PTA-120639 and a VL encoded by the nucleic acid sequence of the insert of the vector deposited as 1 D1 1.31 VL having ATCC accession number PTA-120640.

In some aspects of the disclosure, the anti-TL1A antibody competes for binding with an anti-TL1A antibody comprising a variable heavy chain region having the sequence shown in SEQ ID NO: 1 and a variable light chain region having the sequence shown in SEQ ID NO: 2.

In some aspects of the disclosure, the anti-TL1A antibody competes for binding with an antibody comprising a VH encoded by the nucleic acid sequence of the insert of the vector deposited as 1D1 1.31 VH having ATCC accession number PTA-120639 and a VL encoded by the nucleic acid sequence of the insert of the vector deposited as 1D1 1.31 VL having ATCC accession number PTA-120640.

In some aspects of the disclosure, the anti-TL1A antibody comprises sequence pairs selected from the group consisting of SEQ ID NOs: 2 and 11; SEQ ID NOs: 2 and 12; SEQ ID NOs: 2 and 13; SEQ ID NOs: 2 and 14; SEQ ID NOs: 2 and 15; SEQ ID NOs: 2 and 16; SEQ ID NOs: 2 and 17; SEQ ID NOS: 2 and 18; SEQ ID NOs: 2 and 19; SEQ ID NOs: 20 and 24; SEQ ID NOs: 21 and 25; SEQ ID NOs: 22 and 26; SEQ ID NOs: 23 and 27; SEQ ID NOs: 28 and 29; SEQ ID NOs: 30 and 31; SEQ ID NOs: 32 and 33; SEQ ID NOs: 35 and 44; SEQ ID NOs: 53 and 54; SEQ ID NOs: 61 and 62; SEQ ID NOs: 63 and 64; SEQ ID NOs: 65 and 64; SEQ ID NOs: 66 and 64; and SEQ ID NOs: 67 and 64.

In some aspects of the disclosure, the anti-TL1A antibody comprises a CDR-H1 having the sequence shown in SEQ ID NO: 37, a CDR-H2 having the sequence shown in SEQ ID NO: 39, a CDR-H3 having the sequence shown in SEQ ID NO: 41, a CDR-L1 having the sequence shown in SEQ ID NO: 46, a CDR-L2 having the sequence shown in SEQ ID NO: 48, and a CDR-L3 having the sequence shown in SEQ ID NO: 50.

In some aspects of the disclosure, the anti-TL1A antibody comprises heavy chain framework regions as shown in SEQ ID NOs: 36, 38, 40, and 42 and/or comprises light chain framework regions as shown in SEQ ID NOs: 45, 47, 49, and 51.

In some aspects of the disclosure, the anti-TL1A antibody comprises a heavy chain variable region having the sequence shown in SEQ ID NO: 53 and a light chain variable region having the sequence shown in SEQ ID NO: 54.

In some aspects of the disclosure, the anti-TL1A antibody comprises a heavy chain tail sequence as provided in SEQ ID NO: 43. In some aspects of the disclosure, the anti-TL1A antibody comprises a light chain tail sequence as provided in SEQ ID NO: 52.

In some aspects of the disclosure, the anti-TL1A antibody comprises a heavy chain having the sequence shown in SEQ ID NO: 35 and/or a light chain having the sequence shown in SEQ ID NO: 44.

In some aspects of the disclosure, the anti-TL1A antibody comprises a heavy chain having the sequence shown in SEQ ID NO: 35 and a light chain having the sequence shown in SEQ ID NO: 44.

In some aspects of the disclosure, the anti-TL1A antibody is tulisokibart.

In some aspects, the antibody used in any of the methods, compositions, uses, and compositions for use provided herein is an anti-TL1A antibody provided in Table 2A of U.S. Pat. No. 11,136,386, which is incorporated herein by reference in its entirety.

In some aspects, the antibody used in any of the methods, compositions, uses, and compositions for use provided herein is an anti-DR3 antibody provided in Table 2A of U.S. Pat. No. 11,136,386, which is incorporated herein by reference in its entirety.

In some aspects of any of the methods, compositions, uses, and compositions for use provided herein, the anti-TL1A antibody is an anti-TL1A antibody provided in U.S. Pat. Nos. 10,322,174, 10,689,439, 11,292,848, 10,138,296, 10,822,422, and 11,220,549, which are incorporated herein by reference in their entirety.

In some embodiments, the anti-TL1A antibody comprises the CDR sequences of the 320-179 clone provided in U.S. Pat. No. 10,689,439. In some embodiments, the anti-TL1A antibody is the 320-179 clone provided in U.S. Pat. No. 10,689,439.

In some aspects of the disclosure, the anti-TL1A antibody comprises a CDR-H1 having the sequence shown in SEQ ID NO: 55, a CDR-H2 having the sequence shown in SEQ ID NO: 56, a CDR-H3 having the sequence shown in SEQ ID NO: 57, a CDR-L1 having the sequence shown in SEQ ID NO: 58, a CDR-L2 having the sequence shown in SEQ ID NO: 59, and a CDR-L3 having the sequence shown in SEQ ID NO: 60.

In some aspects of the disclosure, the anti-TL1A antibody comprises a heavy chain variable region having the sequence shown in SEQ ID NO: 61 and a light chain variable region having the sequence shown in SEQ ID NO: 62.

In some aspects of the disclosure, the anti-TL1A antibody comprises a heavy chain having the sequence shown in SEQ ID NO: 63, 65, 66, or 67 and/or a light chain having the sequence shown in SEQ ID NO: 64. In some aspects of the disclosure, the anti-TL1A antibody comprises a heavy chain having the sequence shown in SEQ ID NO: 63, 65, 66, or 67 and a light chain having the sequence shown in SEQ ID NO: 64. In some aspects of the disclosure, the anti-TL1A antibody comprises a heavy chain having the sequence shown in SEQ ID NO: 63 and a light chain having the sequence shown in SEQ ID NO: 64. In some aspects of the disclosure, the anti-TL1A antibody comprises a heavy chain having the sequence shown in SEQ ID NO: 65 and a light chain having the sequence shown in SEQ ID NO: 64. In some aspects of the disclosure, the anti-TL1A antibody comprises a heavy chain having the sequence shown in SEQ ID NO: 66 and a light chain having the sequence shown in SEQ ID NO: 64. In some aspects of the disclosure, the anti-TL1A antibody comprises a heavy chain having the sequence shown in SEQ ID NO: 67 and a light chain having the sequence shown in SEQ ID NO: 64.

In some aspects of the disclosure, the anti-TL1A antibody is TEV-48574.

In some aspects of the disclosure, the anti-TL1A antibody is C03V.

In some aspects of the disclosure, the anti-TL1A antibody is SPY002.

The antibodies as described herein can be made by any method known in the art. For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject disclosure. The hybridomas are expanded and subcloned, if desired, and supernatants are assayed for anti immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies.

Hybridomas that produce antibodies used for the present disclosure may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target (e.g., PD-1), a human target protein (e.g., PD-1), or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC, or R1N═C═NR, where R and R1 are different alkyl groups, can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., J. Immunol. Methods 329, 112, 2008; U.S. Pat. No. 7,314,622.

In some aspects, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

In some aspects, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of b(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art.

### V. Polynucleotides, Vectors and Host Cells

The disclosure also provides polynucleotides encoding any of the anti-TL1A antibodies as described herein. Polynucleotides can be made and expressed by procedures known in the art.

In another aspect, the disclosure provides compositions (such as a pharmaceutical compositions) comprising any of the polynucleotides of the disclosure, for use in one or more methods of the disclosure. In some aspects, the composition comprises an expression vector comprising a polynucleotide encoding any of the anti-TL1A antibodies described herein, for use in one or more methods of the disclosure.

In another aspect, provided is an isolated cell line that produces the anti-TL1A antibodies as described herein for use in one or more methods of the disclosure.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

### VI. Compositions and Formulations

### A. Compositions

The disclosure also provides pharmaceutical compositions comprising an effective amount of an anti-TL1A antibody as described herein, and such pharmaceutical compositions for use in methods of treatment as described herein. Examples of such compositions, as well as how to formulate, are also described herein. It is understood that the compositions can comprise more than one anti-TL1A antibody.

The composition used in the present disclosure can further comprise pharmaceutically acceptable carriers, excipients, or stabilizers (Remington: The Science and practice of Pharmacy 20th Ed., 2000, Lippincott Williams and WIkins, Ed. K. E. Hoover), in the form of lyophilized formulations or aqueous solutions.

Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The anti-TL1A antibody, and compositions thereof, can also be used in conjunction with, or administered separately, simultaneously, or sequentially with other agents that serve to enhance and/or complement the effectiveness of the agents.

### B. Formulations

Therapeutic formulations of the anti-TL1A antibody used in accordance with the present disclosure are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes that may contain the anti-TL1A antibody are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic anti-TL1A antibody compositions are generally placed into a container having a sterile access port, for example, an intravenous solution bag or vial having a stopper pierceable by a hypodermic injection needle.

The compositions according to the present disclosure may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion.

Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an anti-TL1A antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some aspects, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

In aspects that refer to a method of treating IBD as described herein, such aspects are also further aspects of an anti-TL1A antibody for use in that treatment, or alternatively of the use of an anti-TL1A antibody in the manufacture of a medicament for use in that treatment.

### VII. General Techniques

The practice of the present disclosure employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### VIII. Definitions

The following terms, unless otherwise indicated, shall be understood to have the following meanings:

As used herein, “afimkibart” (also known as RO7790121, RVT-3101, or PF-06480605) is an antibody that binds tumor necrosis factor (TNF) superfamily protein TNF-like 1A (TL1A) and comprises the heavy chain sequence of SEQ ID NO: 9 and the light chain sequence of SEQ ID NO: 10.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAi and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain aspects, definitive delineation of a CDR and identification of residues comprising the binding site of an antibody is accomplished by solving the structure of the antibody and/or solving the structure of the antibody-ligand complex. In certain aspects, that can be accomplished by any of a variety of techniques known to those skilled in the art, such as X-ray crystallography. In certain aspects, various methods of analysis can be employed to identify or approximate the CDR regions. In certain aspects, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28:214-8. The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK;

Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, aspects wherein phosphate is replaced by P(0)S(“thioate”), P(S)S (“dithioate”), (0)NR2 (“amidate”), P(0)R, P(0)OR′, CO or CH2 (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. The preceding description applies to all polynucleotides referred to herein, including RNA and DNA.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a target (e.g., TL1A) epitope is an antibody that binds this epitope with greater affinity, avidity, more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-cornpete” with each other for binding of their respective epitope(s). Both competing and cross-competing antibodies are encompassed by the present disclosure. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining beneficial or desired clinical results. For purposes of this disclosure, beneficial or desired clinical results include reduction or improvement in signs and symptoms of inflammatory bowel disease (IBD) (e.g., ulcerative colitis (UC) or Crohn's disease (CD)), for example as compared to before administration of the anti-TL1A antibody.

“Ameliorating” means a lessening or improvement of one and more signs or symptoms of IBD (e.g., UC or CD), for example as compared to not administering an anti-TL1A antibody as described herein. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates signs or symptoms of IBD, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more signs or symptoms of IBD, decreasing the dose of other medications required to treat the disease, enhancing the effect of another medication, and/or delaying the progression of the disease in patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

Treatment “effectively improves” or “effectively reduces” when assessment of the sign or symptom of IBD is quantified via a clinical measure relative to baseline and during and/or after the treatment period. The difference between the clinical measure at baseline and during/after treatment is compared and used to determine whether the sign or symptom has improved and the treatment is effective. This comparison can include comparison to placebo or to one or more of the prior therapies.

The term “mucosal healing” refers to a Mayo endoscopy subscore 0 or 1 and Geboes histology score 0 or 1. Aranzazu, J-E., et al. Journal of Crohn's and Colitis, Volume 11(3), 2017, 305-313. In an aspect, mucosal healing was defined as endoscopic improvement+histologic improvement (Mayo endoscopic subscore <=1 and Geboes <=3.1). Histologic Improvement was defined as <5% crypt involvement of neutrophils in epithelium (Geboes <=3.1). Histologic Remission was defined as no crypt involvement in epithelium (Geboes <2).

Herein, an “effective amount” refers to the amount of a therapeutic agent (e.g., an anti-TL1A antibody (e.g., RO7790121) or a combination of therapeutic agents (e.g., an anti-TL1A antibody and one or more additional therapeutic agents)), that achieves a therapeutic result. In some examples, the effective amount of a therapeutic agent or a combination of therapeutic agents is the amount of the agent or of the combination of agents that achieves a clinical endpoint of clinical remission, clinical response (e.g., improved modified Mayo score (mMS) or partial modified Mayo score (pmMS)), endoscopic improvement, endoscopic remission, histologic-endoscopic mucosal improvement, histologic-endoscopic remission, and/or corticosteroid-free remission. Improvement (e.g., in terms of clinical remission) may be relative to a suitable reference treatment, for example, treatment that does not include the anti-TL1A antibody.

As used herein, the term “induction phase” refers to a series of one or more doses or dosing cycles (e.g., about 2-6 doses or dosing cycles) of one or more therapeutic agents (e.g., an anti-TL1A antibody (e.g., RO7790121) administered to a subject, wherein the one or more doses or dosing cycles are optionally followed by a maintenance phase.

The term “maintenance phase” as used herein refers to a series of one or more doses or dosing cycles of one or more therapeutic agents (e.g., an anti-TL1A antibody (e.g., RO7790121) that are administered to a subject subsequent to an induction phase with no relevant intervening surgery (i.e., no intervening surgery relating to the disease or condition intended to be treated by the one or more therapeutic agents). In some instances, the maintenance phase is initiated only if the subject did not experience disease progression or unacceptable toxicity during the induction phase. The induction phase and maintenance phase may or may not comprise use of the same therapeutic agents.

A “patient”, an “individual” or a “subject”, used interchangeably herein, is a mammal, more preferably, a human. Mammals also include, but are not limited to, farm animals (e.g., cows, pigs, horses, chickens, etc.), sport animals, pets, primates, horses, dogs, cats, mice and rats.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

Reference to “about” a value or parameter herein includes (and describes) aspects that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The term “subcutaneous administration” refers to the administration of a substance into the subcutaneous layer.

The term “preventing” or “prevent” refers to (a) keeping a disorder from occurring or (b) delaying the onset of a disorder or onset of symptoms of a disorder.

It is understood that wherever aspects are described herein with the language “comprising,” otherwise analogous aspects described in terms of “consisting of” and/or “consisting essentially of” are also provided.

Where aspects or aspects of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claimed disclosure.

Unless otherwise defined, all technical and scientific terms used herein have the same meaning as commonly understood by one of ordinary skill in the art to which this disclosure belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### IX. Examples

### Example 1. TNFSF15 Haplotype SNP Combinations

In this experiment, a computational algorithm was developed to determine, based on DNA sequencing data (e.g., genome sequencing data), whether a subject is a carrier for a TNFSF15 haplotype that is haplotype B (i.e., whether a subject is a haplotype B carrier) based on a selected number of single-nucleotide polymorphisms (SNPs).

As shown in FIG. 1, haplotype B is defined as a TNFSF15 gene region that has the nucleotides T/C/A/T/C at position 15,524; position 9,706; position-358; position-638; and position-12,506, respectively, based on the reference sequence GenBank NM_005118.2, wherein the first nucleotide of the exon 1 start site is designated as position 1 (haplotype positions 26/31/35/36/41, respectively, as provided in Yamazaki et al., Human Molecular Genetics, 14 (22): 3499-3506, 2005), in the TNFSF15 gene region, which encodes the TL1A gene (Table 3). A subject is identified as haplotype B if the subject is heterozygous or homozygous for haplotype B, i.e., one or both copies of the gene region are haplotype B (i.e., one or both of the TNFSF15 gene regions have the nucleotides T/C/A/T/C at haplotype positions 15,524; 9,706; -358; -638; and -12,506 (26/31/35/36/41).

Haplotype positions 15,524; 9,706; -358; -638; and -12,506 (26, 31, 35, 36 and 41) correspond to the sites of the SNPs rs3810936, rs6478108, rs6478109, rs7848647, and rs7869487, respectively, as identified in the NIH NCBI dbSNP database (see Table 3). Haplotype B comprises the reference nucleotide at each of these positions: thus, a haplotype B TNFSF15 gene region can be identified by the absence of each of SNPs rs3810936, rs6478108, rs6478109, rs7848647, and rs7869487 (i.e., the presence of the wild-type allele at each of these sites).

Haplotype B non-carrier subjects are defined as subjects that do not carry a haplotype B TNFSF15 gene region. For example, a haplotype B non-carrier subject may be homozygous for a non-haplotype B allele, e.g., haplotype A as shown in FIG. 1 (Michelsen et al., PLoS One, 4(3): e4719, 2009) (e.g., may have the nucleotides C/T/G/C/T at haplotype positions 26/31/35/36/41).

TABLE 3
TNFSF15 gene region SNPs of interest

| | | Position number | | | | | |
|---|---|---|---|---|---|---|---|
| | | relative to GenBank | | | | | |
| | Position | NM_005118.2, wherein | | | | | |
| | number | the first nucleotide | NIH | | | | |
| | according to | of the exon 1 start | Reference | Position on | | | |
| | Yamazaki | site is designated | SNP (rs) | human genome | Reference | | Haplotype B |
| SNP | et al., 2005 | as position 1 | identifier | build hg38 | nucleotide | SNP | nucleotide |
| SNP 1 | 26 | 15,524 | rs3810936 | chr9: 114790605 | T | C | T |
| SNP 2 | 31 | 9,706 | rs6478108 | chr9: 114796423 | C | G or T | C |
| SNP 3 | 35 | −358 | rs6478109 | chr9: 114806486 | A | G | A |
| SNP 4 | 36 | −638 | rs7848647 | chr9: 114806766 | T | C | T |
| SNP 5 | 41 | −12,506 | rs7869487 | chr9: 114818634 | C | T | C |

In the development of the algorithm, different combinations of five SNP sites, rs3810936 (SNP 1), rs6478108 (SNP 2), rs6478109 (SNP 3), rs7848647 (SNP 4), and rs7869487 (SNP5), including all viable combinations of 2, 3, and 4 SNPs selected from SNP 1, SNP 2, SNP 3, SNP 4 and SNP 5, were used to call whether a subject was a haplotype B carrier or a haplotype B non-carrier. A subject was defined as a haplotype B non-carrier if both copies of the gene region corresponding to any one of the five SNP sites did not contain the nucleotide corresponding to the haplotype B nucleotide at the SNP site, e.g., CC/TT/GG/CC/TT at SNP1-SNP5. For each SNP combination, if the first SNP site from the combination tested was of a haplotype other than haplotype B in both copies of the gene region (i.e., referred hereafter as “Not Haplotype B homozygous”), then the subject was determined to be a haplotype B non-carrier. If the first SNP site from the combination tested was haplotype B homozygous or heterozygous (i.e., if either copy or both copies of the gene region corresponding to the SNP site contained the nucleotide corresponding to the haplotype B nucleotide at the SNP site), then the next SNP site in the combination was tested, until either one SNP site was determined to be Not Haplotype B homozygous, in which case, the subject was determined to be a haplotype B non-carrier, or all SNP sites in the combination were tested. If no SNP sites were determined to be Not Haplotype B homozygous, the subject was determined to be a haplotype B carrier. The final step of this algorithm was where misclassifications may occur. Therefore, testing all SNP combinations allowed determination of which SNP combination yielded the lowest number of misclassifications.

Iterations of the algorithm comprising different SNP combinations were applied to the genome sequence data (a total of 2504 subjects) from the 1000 Genomes Project (Sudmant et al., Nature 526, 75-81 (2015)), and the true positive, true negative, false positive and false negative rates were determined for each SNP combination in order to determine the optimal combination of SNPs that can effectively identify haplotype B carriers and haplotype B non-carriers. As shown in Table 4, the combination of SNP 1 (rs3810936) and SNP 5 (rs7869487) provided the highest accuracy among the various two-SNP-site combinations, and the accuracy rate is on par with the best performing three-SNP-site and four-SNP-site combinations.

TABLE 4
Accuracy of SNP combinations in determining whether a subject
is a TNFSF15 haplotype B carrier (1000 Genomes Project data)

| SNP | True | True | False | False | Number | Number | Percent | Percent |
|---|---|---|---|---|---|---|---|---|
| combination | Positive | Negative | Positive | Negative | Correct | Incorrect | Correct | Incorrect |
| (1, 2) | 1444 | 889 | 0 | 171 | 2333 | 171 | 0.931709265 | 0.068290735 |
| (1, 3) | 1456 | 889 | 0 | 159 | 2345 | 159 | 0.936501597 | 0.063498403 |
| (1, 4) | 1455 | 889 | 0 | 160 | 2344 | 160 | 0.936102236 | 0.063897764 |
| (1, 5) | 1595 | 889 | 0 | 20 | 2484 | 20 | 0.99201278 | 0.00798722 |
| (2, 1) | 1444 | 889 | 0 | 171 | 2333 | 171 | 0.931709265 | 0.068290735 |
| (2, 3) | 1368 | 889 | 0 | 247 | 2257 | 247 | 0.901357827 | 0.098642173 |
| (2, 4) | 1365 | 889 | 0 | 250 | 2254 | 250 | 0.900159744 | 0.099840256 |
| . . . | . . . | . . . | . . . | . . . | . . . | . . . | . . . | . . . |
| (5, 3, 2) | 1533 | 889 | 0 | 82 | 2422 | 82 | 0.967252396 | 0.032747604 |
| (5, 3, 4) | 1534 | 889 | 0 | 81 | 2423 | 81 | 0.967651757 | 0.032348243 |
| (5, 4, 1) | 1603 | 889 | 0 | 12 | 2492 | 12 | 0.995207668 | 0.004792332 |
| (5, 4, 2) | 1533 | 889 | 0 | 82 | 2422 | 82 | 0.967252396 | 0.032747604 |
| (5, 4, 3) | 1534 | 889 | 0 | 81 | 2423 | 81 | 0.967651757 | 0.032348243 |
| (1, 2, 3, 4) | 1456 | 889 | 0 | 159 | 2345 | 159 | 0.936501597 | 0.063498403 |
| (1, 2, 3, 5) | 1604 | 889 | 0 | 11 | 2493 | 11 | 0.995607029 | 0.004392971 |
| (1, 2, 4, 3) | 1456 | 889 | 0 | 159 | 2345 | 159 | 0.936501597 | 0.063498403 |
| (1, 2, 4, 5) | 1603 | 889 | 0 | 12 | 2492 | 12 | 0.995207668 | 0.004792332 |
| (1, 2, 5, 3) | 1604 | 889 | 0 | 11 | 2493 | 11 | 0.995607029 | 0.004392971 |

The algorithm was further applied to the genome sequence data (a total of 4151 samples, with 2501 samples overlapping with the 1000 Genome Project) from the Human Genome Diversity Project (Bergstrom et al., Science 367 (6484), eaay5012 (2020)). The data from the Human Genome Diversity Project was not phased, unlike the 1000 Genomes data, and the misclassified subjects in the overlapping set of data have different truth values due to phasing. Table 5 shows results from the analysis of the Human Genome Diversity Project. Notably, the three-SNP-site combination of SNP1 (rs3810936), SNP 3 (rs6478109) and SNP 5 (rs7869487) yielded 0 misclassifications. Table 6 shows accuracy rates for certain SNP site combinations evaluated using the Human Genome Diversity Project.

TABLE 5
Accuracy of SNP combinations in determining whether a subject is a
TNFSF15 haplotype B carrier (Human Genome Diversity Project data)

| SNP | True | True | False | False | Number | Number | Percent | Percent |
|---|---|---|---|---|---|---|---|---|
| combination | Positive | Negative | Positive | Negative | Correct | Incorrect | Correct | Incorrect |
| (1, 2) | 2408 | 1470 | 0 | 273 | 3878 | 273 | 0.934232715 | 0.065767285 |
| (1, 3) | 2425 | 1470 | 0 | 256 | 3895 | 256 | 0.938328114 | 0.061671886 |
| (1, 4) | 2425 | 1470 | 0 | 256 | 3895 | 256 | 0.938328114 | 0.061671886 |
| (1, 5) | 2668 | 1470 | 0 | 13 | 4138 | 13 | 0.996868225 | 0.003131775 |
| (2, 1) | 2408 | 1470 | 0 | 273 | 3878 | 273 | 0.934232715 | 0.065767285 |
| (2, 3) | 2291 | 1470 | 0 | 390 | 3761 | 390 | 0.906046736 | 0.093953264 |
| (2, 4) | 2288 | 1470 | 0 | 393 | 3758 | 393 | 0.905324018 | 0.094675982 |
| . . . | . . . | . . . | . . . | . . . | . . . | . . . | . . . | . . . |
| (1, 3, 5) | 2681 | 1470 | 0 | 0 | 4151 | 0 | 1 | 0 |
| . . . | . . . | . . . | . . . | . . . | . . . | . . . | . . . | . . . |
| (5, 3, 2) | 2575 | 1470 | 0 | 106 | 4045 | 106 | 0.974463985 | 0.025536015 |
| (5, 3, 4) | 2576 | 1470 | 0 | 105 | 4046 | 105 | 0.97470489 | 0.02529511 |
| (5, 4, 1) | 2680 | 1470 | 0 | 1 | 4150 | 1 | 0.999759094 | 0.000240906 |
| (5, 4, 2) | 2575 | 1470 | 0 | 106 | 4045 | 106 | 0.974463985 | 0.025536015 |
| (5, 4, 3) | 2576 | 1470 | 0 | 105 | 4046 | 105 | 0.97470489 | 0.02529511 |
| (1, 2, 3, 4) | 2426 | 1470 | 0 | 255 | 3896 | 255 | 0.93856902 | 0.06143098 |
| (1, 2, 3, 5) | 2681 | 1470 | 0 | 0 | 4151 | 0 | 1 | 0 |
| (1, 2, 4, 3) | 2426 | 1470 | 0 | 255 | 3896 | 255 | 0.93856902 | 0.06143098 |
| (1, 2, 4, 5) | 2680 | 1470 | 0 | 1 | 4150 | 1 | 0.999759094 | 0.000240906 |
| (1, 2, 5, 3) | 2681 | 1470 | 0 | 0 | 4151 | 0 | 1 | 0 |

TABLE 6
Accuracy rates of SNP combinations in determining whether a subject
is a TNFSF15 haplotype B carrier (Human Genome Project data)

| SNP Combination | Portion Correct | Number of SNPs assessed |
|---|---|---|
| (1, 3, 5) | 0.9998 | 3 |
| (1, 3, 4, 5) | 0.9998 | 4 |
| (1, 2, 3, 5) | 0.9998 | 4 |
| (1, 2, 4, 5) | 0.9995 | 4 |
| (1, 4, 5) | 0.9995 | 3 |
| (1, 2, 5) | 0.9966 | 3 |
| (1, 5) | 0.9966 | 2 |
| (3, 4, 5) | 0.9745 | 3 |
| (2, 3, 4, 5) | 0.9745 | 4 |
| (4, 5) | 0.9742 | 2 |
| (3, 5) | 0.9742 | 2 |
| (2, 3, 5) | 0.9742 | 3 |
| (2, 4, 5) | 0.9742 | 3 |
| (2, 5) | 0.9639 | 2 |
| (1, 2, 3) | 0.9383 | 3 |
| (1, 3, 4) | 0.9383 | 3 |
| (1, 2, 3, 4) | 0.9383 | 4 |
| (1, 2, 4) | 0.9381 | 3 |
| (1, 3) | 0.9381 | 2 |
| (1, 4) | 0.9381 | 2 |
| (1, 2) | 0.9340 | 2 |
| (2, 3, 4) | 0.9063 | 3 |
| (2, 3) | 0.9058 | 2 |
| (2, 4) | 0.9051 | 2 |
| (3, 4) | 0.9046 | 2 |

### Example 2. 2-SNP Multiplex qPCR Assay

The TL1A SNP Test is a multiplexing qPCR-based assay that detects the presence of two specific germline SNPs, rs3810936 (SNP 1) and rs7869487 (SNP 5), which identify TNFSF15 haplotype B non-carrier patients who may potentially obtain additional benefit from afimkibart therapy (see Example 1), in a biological sample from the patient. In the assay, short DNA targets (amplicons) are exponentially amplified over multiple cycles. Fluorescently labeled probes designed to bind to amplicons allow for appropriate discrimination between the presence of a SNP (Mutant/MUT; also referred to as “alternate: or “Alt”) or absence of a SNP (Wildtype/WT; “Ref”). A sample with mixed amplicons will have a mix of WT and MUT probe binding to the amplicons, thus allowing for discrimination of Heterozygous (HET) samples. The probes are constructed with a reporter dye (FAM, SUN, TAMRA, and Cy5) on the 5′ end and a quencher dye (BHQ-1 and BHQ-2) on the 3′ end. While the probe is intact, the proximity of the quencher dye greatly reduces the fluorescence emitted by the reporter dye by fluorescence resonance energy transfer (FRET). If the target sequence is present, the probe anneals to its specific complement region in the amplicon and is cleaved by the 5′ nuclease activity of Taq DNA polymerase as this primer is extended. The cleavage separates the reporter dye from the quencher dye, resulting in increased signal of the reporter dye. Additional reporter dyes are cleaved from their probes with each cycle, resulting in an increase in fluorescence intensity proportional to the amount of amplicon produced. The fluorescence signal is monitored after each cycle and the proprietary auto calling feature of the QUANTSTUDIO™ 5 Dx software determines the genotype calls.

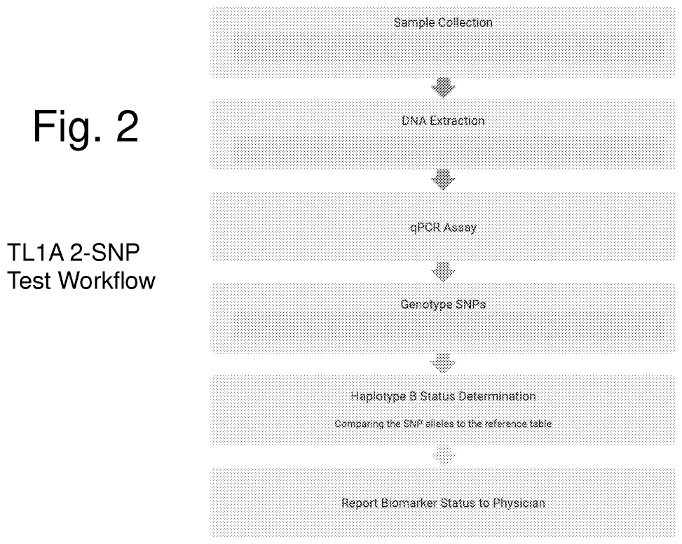

Once the genotypes of the two target SNP sites have been identified, the haplotype B carrier/non-carrier status of the patient can be determined by comparing the SNP alleles to the reference table provided below (see Table 7 and FIG. 2). For rs3810936 (SNP 1), the reference (“Ref”) allele is T, and the alternate (“Alt”) allele is C. For 7869487 (SNP 5), the reference (“Ref”) allele is C, and the alternate (“Alt”) allele is T.

Patients identified as haplotype B carriers are classified as having a negative biomarker status. Patients identified as haplotype B non-carriers are classified as having a positive biomarker status (i.e., are identified as patients who may potentially obtain additional benefit from afimkibart therapy).

TABLE 7
TNFSF15 Biomarker Status Reference Table

| SNP 1 | SNP 5 | Haplotype B | Biomarker |
|---|---|---|---|
| rs3810936 (T > C) | rs7869487 (C > T) | Carrier Status | Status |
| Ref:Ref | Ref:Ref | Carrier | Negative |
| Ref:Ref | Ref:Alt | Carrier | Negative |
| Ref:Ref | Alt:Alt | Noncarrier | Positive |
| Ref:Alt | Ref:Ref | Carrier | Negative |
| Ref:Alt | Ref:Alt | Carrier/ | Negative² |
| | | Noncarrier¹ | |
| Ref:Alt | Alt:Alt | Noncarrier | Positive |
| Alt:Alt | Ref:Ref | Noncarrier | Positive |
| Alt:Alt | Ref:Alt | Noncarrier | Positive |
| Alt:Alt | Alt:Alt | Noncarrier | Positive |

¹Based on 1000 Genomes Project database, 97.3% of subjects in these categories are haplotype B carriers (see Example 1).
²Presumed biomarker-negative based on overall misclassification rate of ~1% from 1000 Genomes Project database (see Example 1).

### Example 3. Efficacy and Safety of RO7790121, a Fully Human Monoclonal Antibody Blocking TL1A in Moderately to Severely Active Ulcerative Colitis: Results from a Randomized, Double-Blind, Placebo-Controlled Dose-Ranging Phase 2b TUSCANY-2 Study

### Introduction

Tumor necrosis factor-like ligand 1A (TL1A) is a novel therapeutic target for inflammatory bowel disease. Patients with moderately to severely active ulcerative colitis (UC) treated with the anti-TL1A antibody afimkibart (RO7790121) (a fully human, neutralizing monoclonal antibody against TL1A) showed significant endoscopic improvement and adequate safety in the phase 2a TUSCANY-1 trial (Danese et al., Clin Gastroenterol Hepatol, 19:2324-32.e6, 2021).

This Example provides data from the global, randomized, double-blind, placebo-controlled dose-ranging Phase 2b TUSCANY-2 (NCT04090411) trial evaluating the efficacy and safety of various subcutaneous (SC) doses of afimkibart administered every four weeks (Q4W) in male or female patients aged 18-75 years with moderately to severely active UC (total Mayo Score ≥6, endoscopic subscore ≥2) who had previously failed or been intolerant to ≥1 line of conventional or advanced treatment (selected from the following classes: steroids, immunosuppressants, anti-TNFs, anti-integrin inhibitors, anti-IL-12/23 inhibitors, and JAK inhibitors).

### Aims and Methods

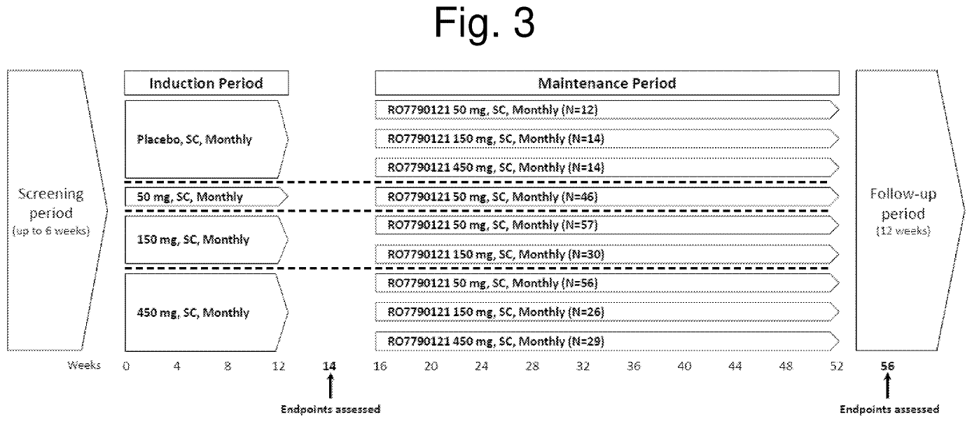

Patients were randomized to receive afimkibart SC 50 mg, 150 mg, 450 mg, or matched placebo (PBO) monthly during a 12-week induction period and afimkibart SC 50, 150, or 450 mg monthly during a treat-through 40-week maintenance period (FIG. 3). Efficacy endpoints were assessed at Weeks 14 and 56 in the induction and maintenance intent-to-treat analysis sets, respectively. The primary endpoints were clinical remission at week 14 by total Mayo Score (MS) and safety at Weeks 14 and 56. The prespecified key secondary endpoint of clinical remission using modified Mayo Score (mMS) is aligned to updated FDA guidance. Other secondary endpoints included clinical remission (by total MS and mMS) at week 56 and endoscopic improvement at Weeks 14 and 56. Efficacy analysis by pre-defined biomarker subgroups was also performed.

Table 8 provides definitions of efficacy endpoints of the TUSCANY-2 study.

TABLE 8
Definition of efficacy endpoints

| Endpoint | Definition |
|---|---|
| Clinical remission | Total Mayo Score ≤2, with no individual subscore >1. |
| Clinical remission 2 | Total Mayo score ≤2, rectal bleeding subscore = 0, endoscopic |
| | subscore = 0 or 1, stool frequency subscore = 0 or 1, and |
| | physician's global assessment = 0 or 1. |
| Modified remission 1 | Endoscopic subscore = 0 or 1, stool frequency subscore = 0, and |
| (remission FDA definition 1) | rectal bleeding subscore = 0. |
| Modified remission 2 | Endoscopic subscore = 0 or 1, ≥1 point decrease from baseline to |
| (remission FDA definition 2) | achieve a stool frequency subscore = 0 or 1, and rectal bleeding |
| | subscore = 0. |
| Endoscopic improvement | Endoscopic subscore = 0 or 1. |
| Endoscopic remission | Endoscopic subscore = 0. |
| Clinical response | ≥3 point and ≥30% decrease from baseline in Total Mayo Scores, with |
| | an accompanying ≥1 point decrease in rectal bleeding subscore or |
| | absolute rectal bleeding subscore = 0 or 1. |
| Clinical modified response | ≥3 point and ≥30% decrease from baseline in modified Mayo Scores, |
| | with an accompanying ≥1 point decrease in rectal bleeding subscore or |
| | absolute rectal bleeding subscore = 0 or 1. |
| Symptomatic remission | Total Mayo Score ≤2 with no individual subscore >1 and both rectal |
| | bleeding and stool frequency subscores of 0. |
| Deep remission | Total Mayo Score ≤2 with no individual subscore >1 and both |
| | endoscopic and rectal bleeding subscores of 0. |

“Mayo Score” refers to a Mayo score according to the Mayo Scoring System for Assessment of Ulcerative Colitis Activity.

### Study Design

TUSCANY-2 was a Phase 2b, multi-center, randomized, double-blind, placebo-controlled, parallel group study designed to assess the efficacy, safety and PK of PF-06480605 in participants with moderate to severe active UC. Approximately 240 participants were planned to be randomized, and 216 of them were expected to complete the induction period, assuming a 10% drop out rate. Participants were randomly assigned to 1 of 9 treatment sequences using an allocation ratio of 2:2:2:2:2:3:1:1:1 (Table 9).

TABLE 9
Treatment sequence

| | | | Planned |
|---|---|---|---|
| | | | Participant |
| Treatment | Treatment Sequence Description | | Sample |
| Sequence | Induction Period | Chronic Therapy Period | Size |
| A (450 mg → 450 mg) | PF-06480605 450 mg SC Q4W | PF-06480605 450 mg SC Q4W | 30 |
| B (450 mg → 150 mg) | PF-06480605 450 mg SC Q4W | PF-06480605 150 mg SC Q4W | 30 |
| C (450 mg → 50 mg) | PF-06480605 450 mg SC Q4W | PF-06480605 50 mg SC Q4W | 30 |
| D (150 mg → 150 mg) | PF-06480605 150 mg SC Q4W | PF-06480605 150 mg SC Q4W | 30 |
| E (150 mg → 50 mg) | PF-06480605 150 mg SC Q4W | PF-06480605 50 mg SC Q4W | 30 |
| F (50 mg → 50 mg) | PF-06480605 50 mg SC Q4W | PF-06480605 50 mg SC Q4W | 45 |
| G (placebo → 450 mg) | Placebo SC Q4W | PF-06480605 450 mg SC Q4W | 15 |
| H (placebo → 150 mg) | Placebo SC Q4W | PF-06480605 150 mg SC Q4W | 15 |
| I (placebo → 50 mg) | Placebo SC Q4W | PF-06480605 50 mg SC Q4W | 15 |

SC = subcutaneous; Q4W = every 4 weeks.

The study occurred over 4 periods; screening, induction, chronic therapy and follow-up. The screening period lasted up to 6 weeks to assess eligibility. During the 12-week induction period, participants were randomized to receive 50 mg, 150 mg, 450 mg, or matched placebo by subcutaneous administration every 4 weeks. During the 40-week chronic therapy period, all participants received active drug: 50 mg, 150 mg, or 450 mg by subcutaneous administration every 4 weeks. During the 12-week follow-up period, final safety and efficacy assessments were planned. Overall, the study participation for each participant was up to 70 weeks. Participants that discontinued at any time during the study proceeded into the follow-up period with an early withdrawal visit. Any participant who prematurely withdrew from the treatment period (after being randomized and during active study intervention) was followed for 3 study visits (one being the Early Withdrawal Visit) for a total of 12 weeks from the last dose of interventional product.

Two interim analyses were conducted for the study. The first analysis was conducted when 100% of participants had completed or had the opportunity to complete the Week 14 Visit. The second interim analysis was conducted when all participants had completed or had the opportunity to complete the induction period and approximately 100% of them had completed or had the opportunity to complete at least 6 months of chronic therapy.

### Results

A total of 246 participants were enrolled at 114 centers in 23 countries. Overall, 245 patients entered induction (received at least one induction dose); 228 patients completed the induction phase, and 224 patients entered the maintenance period of the study (received at least one maintenance dose).

During the induction period, all 245 treated participants (45, 47, 62, and 91 in the placebo, PF-06480605 50 mg, 150 mg, and 450 mg groups, respectively) were included in the safety and intent-to-treat (ITT) analysis population; 240 participants (40, 47, 62, and 91 in the four groups, respectively) were included in the biomarker analysis population; 239 participants (40, 47, 62, and 90 in the four groups, respectively) were included in the immunogenicity analysis population.

During the chronic therapy period, a total of 224 participants (12 participants [placebo→50 mg], 14 participants [placebo→150 mg], 14 participants [placebo→450 mg], 46 participants [50 mg→50 mg], 27 participants [150 mg→50 mg], 30 participants [150 mg→150 mg], 26 participants [450 mg→50 mg], 26 participants [450 mg→150 mg], and 29 participants [450 mg→450 mg]) were included in the maintenance intent-to-treat (mITT) analysis.

### A. Demographics and Baseline Characteristics

Demographics and baseline characteristics of patients treated in the TUSCANY-2 study are shown in Table 10. Demographic characteristics were generally similar among placebo and PF-06480605 groups.

TABLE 10
Demographics and baseline characteristics

| | | | | | |
|---|---|---|---|---|---|
| | Placebo | 50 mg | 150 mg | 450 mg | Overall |
| Induction arm | (N = 45) | (N = 47) | (N = 62) | (N = 91) | (N = 245) |

Age (years)

| | | | | | | | | | | |
|---|---|---|---|---|---|---|---|---|---|---|
| Mean (SD) | 39.9 | (12.90) | 37.8 | (13.91) | 42.2 | (13.02) | 41.6 | (13.79) | 40.7 | (13.48) |
| Median (range) | 38 | (19-66) | 34 | (18-73) | 41 | (20-70) | 40 | (19-73) | 39 | (18-73) |
| Male, n (%) | 24 | (53.3) | 28 | (59.6) | 39 | (62.9) | 55 | (60.4) | 146 | (59.6) |

Race, n (%)

| | | | | | | | | | | |
|---|---|---|---|---|---|---|---|---|---|---|
| White | 30 | (66.7) | 35 | (74.5) | 49 | (79.0) | 70 | (76.9) | 184 | (75.1) |
| Black or African | 1 | (2.2) | 0 | (0.0) | 0 | (0.0) | 0 | (0.0) | 1 | (0.4) |
| American | | | | | | | | | | |
| Asian | 13 | (28.9) | 9 | (19.1) | 9 | (14.5) | 18 | (19.8) | 49 | (20.0) |
| Other or not | 1 | (2.2) | 3 | (6.4) | 4 | (6.5) | 3 | (3.3) | 11 | (4.5) |
| reported | | | | | | | | | | |

Region, n (%)

| | | | | | | | | | | |
|---|---|---|---|---|---|---|---|---|---|---|
| US/Canada/ | 5 | (11.1) | 5 | (10.6) | 4 | (6.5) | 15 | (16.5) | 29 | (11.8) |
| Australia | | | | | | | | | | |
| Europe | 25 | (55.6) | 31 | (66.0) | 47 | (75.8) | 54 | (59.3) | 157 | (64.1) |
| Asia | 13 | (28.9) | 9 | (19.1) | 9 | (14.5) | 17 | (18.7) | 48 | (19.6) |
| Other | 2 | (4.4) | 2 | (4.3) | 2 | (3.2) | 5 | (5.5) | 11 | (4.5) |
| Height (cm), | 167.6 | (151-192) | 171.0 | (154-192) | 170.0 | (149-185) | 172.0 | (146-196) | 170.0 | (146-196) |
| median (range) | | | | | | | | | | |
| Weight (kg), | 66.0 | (50.0-113) | 69.1 | (45.0-130) | 70.6 | (33.9-112) | 68.5 | (37.7-115) | 68.2 | (33.9-130) |
| median (range) | | | | | | | | | | |
| Advanced therapy | 17 | (37.8) | 19 | (40.4) | 21 | (33.9) | 39 | (42.9) | 96 | (39.2) |
| Inadequate | | | | | | | | | | |
| response (IR), | | | | | | | | | | |
| n (%) | | | | | | | | | | |

Baseline total Mayo score (MS)

| | | | | | | | | | | |
|---|---|---|---|---|---|---|---|---|---|---|
| Mean (SD) | 9.0 | (1.8) | 8.6 | (1.5) | 9.0 | (1.6) | 9.3 | (1.5) | 9.0 | (1.6) |
| ≥10, n (%) | 17 | (37.8) | 17 | (36.2) | 29 | (46.8) | 46 | (50.5) | 109 | (44.5) |

Among participants treated during the induction period, the median duration of disease was 4.70 years and ranged from 0.25 to 36.2 years. The baseline disease characteristics were balanced among placebo and the three PF-06480605 groups.

Among participants treated during the induction period, most participants were in the disease extent of left-sided colitis (43.3%), followed by pancolitis (diagnosed in 39.2% of participants). The extent of disease was generally balanced among placebo and the three PF-06480605 groups.

Among participants treated during the induction period, a total of 95.5%, 41.2%, 38.8%, and 23.7% of participants received prior steroid, biologic, anti-TNF, and/or anti-integrin treatment, respectively. The prior medical experience (i.e., steroid, biologic, anti-TNF, anti-integrin, azathioprine, 6-MP, methotrexate, anti-IL-12/23 inhibitors, and JAK inhibitor) was generally balanced among placebo and the three PF-06480605 groups.

Among participants treated during the chronic therapy period, a total of 96.0%, 41.5%, 39.3%, and 24.1% of participants received prior steroid, biologic, anti-TNF, and/or anti-integrin treatment, respectively. The prior medical experience was generally balanced among all treatment sequences.

Among participants treated during the induction period, 97.1% of participants received general concomitant medications. A total of 73.1% of participants received mesalazine (ranging from 67.7% to 80.2% among placebo and the three PF-06480605 groups).

Among participants treated during the chronic therapy period, 76.8% of participants received general concomitant medications.

Among participants treated during the induction period, 90 out of 245 participants used steroids at baseline. Of those 90 participants, 89 (98.9%) did not change steroids dose.

Among participants treated during the chronic therapy period, 86 out of 224 participants used steroids at baseline. Of those 86 participants, 43 (50.0%) did not change steroid dose.

### B. Efficacy

At Week 14, 25.5%, 23.3% and 23.9% of patients in the afimkibart 50 mg, 150 mg, 450 mg arms, respectively vs. 11.6% in the placebo arm experienced clinical remission by total Mayo Score (p-value >0.05 (Table 11).

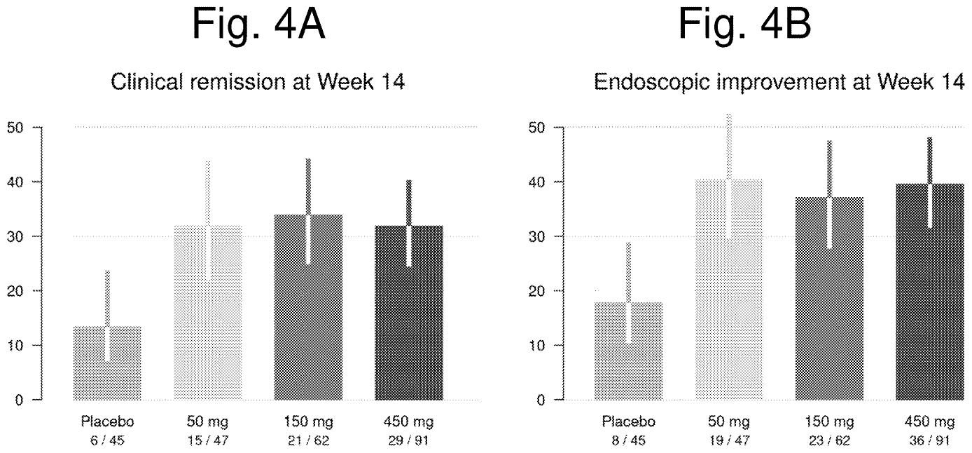

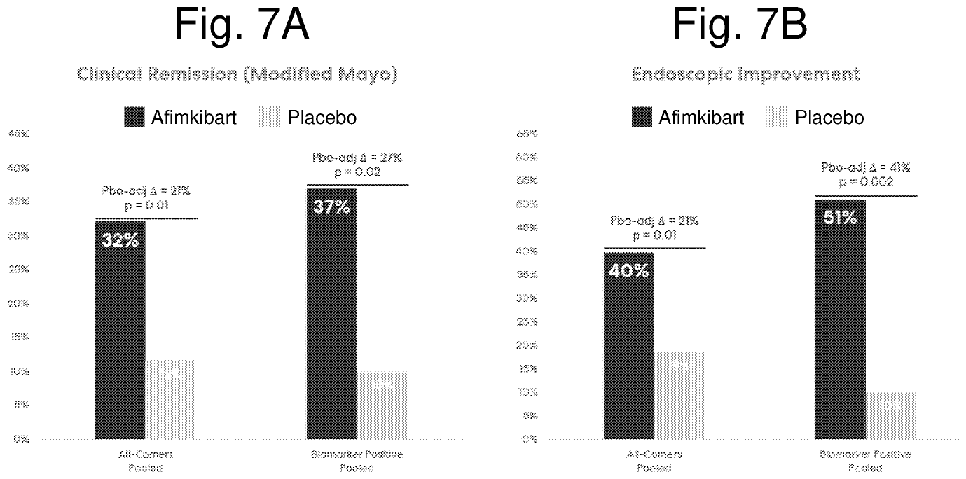

The proportion of patients achieving clinical remission by modified Mayo score (mMS) at Week 14 was 29.8%, 35.0% and 31.8% in the afimkibart 50 mg, 150 mg, 450 mg arms, respectively vs. 11.6% in placebo (p-value <0.05) (Table 11 and FIG. 4A). Consistent, statistically significant effects were observed at Week 14 across endpoints and patient populations. As is shown in FIGS. 7A and 7B, afimkibart achieved rates of clinical remission (according to mMS) and endoscopic improvement greater than placebo in both the all-comer and TNFSF15 biomarker-positive (haplotype B non-carrier; “biomarker-positive”) patient populations.

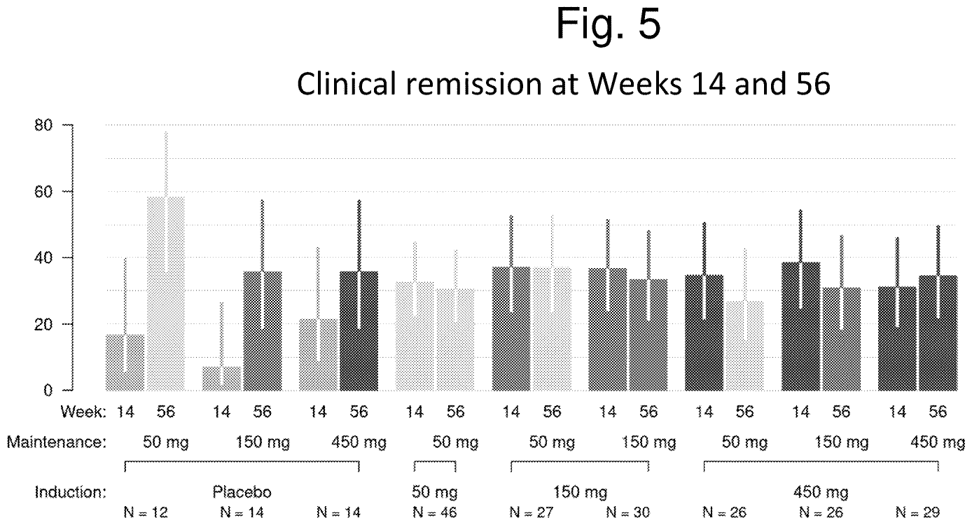

Improvements in clinical remission were sustained through maintenance (Table 11 and FIG. 5).

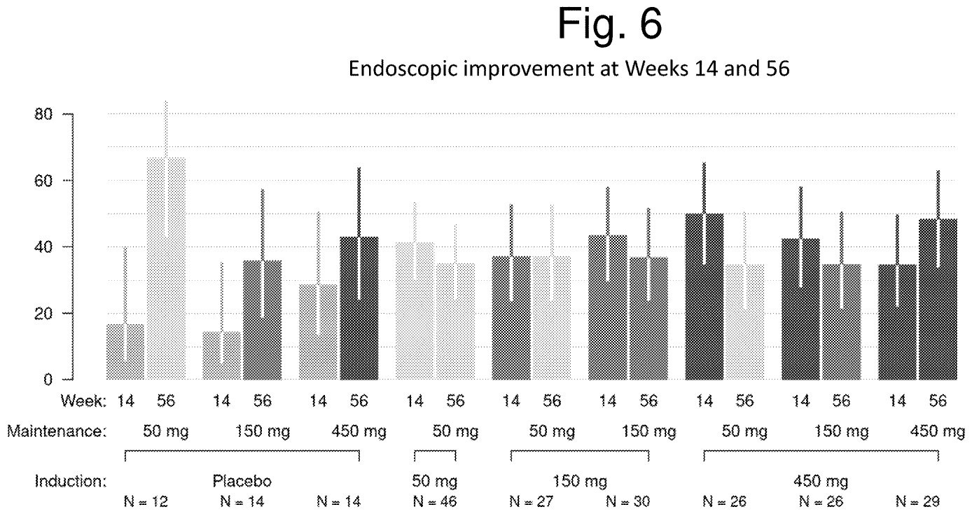

In addition, a larger proportion of patients demonstrated endoscopic improvement across all doses of afimkibart vs. placebo at Week 14 and Week 56 (Table 11 and FIGS. 4B and 6). Trends of numerically higher treatment effects were observed in biomarker-positive patients relative to all-comer patients.

TABLE 11
Summary of efficacy outcomes at Week 14 and Week 56

| | Placebo | 50 mg | 150 mg | 450 mg |
|---|---|---|---|---|
| Induction, Week 14 | (n = 43) | (n = 47) | (n = 60) | (n = 88) |
| Clinical remission by total Mayo | 11.6 | 25.5 | 23.3 | 23.9 |
| Score,* % (90% CI) | (5.8, 22.9) | (15.4, 37.2) | (15.0, 34.0) | (16.6, 32.1) |
| Clinical remission by modified Mayo | 11.6 | 29.8 | 35.0 | 31.8 |
| score (mMS),^{†} % (90% CI) | (5.8, 22.9) | (19.9, 42.3) | (25.1, 45.2) | (23.7, 40.8) |
| Endoscopic improvement,^{‡} % (90% CI) | 18.6 | 40.4 | 38.3 | 40.9 |
| | (9.6, 30.2) | (28.3, 53.5) | (27.8, 48.6) | (32.1, 50.0) |
| | | 50 mg | 150 mg | 450 mg |
| Maintenance, Week 56^{§} | N/A^{¶} | (n = 42) | (n = 26) | (n = 28) |
| Clinical remission by total Mayo | | 31.0 | 34.6 | 39.3 |
| Score,* % (90% CI) | | (19.4, 43.3) | (20.9, 52.6) | (23.8, 56.5) |
| Clinical remission by mMS,^{†} % (90% CI) | | 31.0 | 38.5 | 35.7 |
| | | (19.4, 43.3) | (23.3, 56.4) | (20.9, 52.7) |
| Endoscopic improvement,^{‡} % (90% CI) | | 38.1 | 39.3** | 50.0 |
| | | (25.6, 52.0) | (23.8, 56.5) | (33.3, 66.7) |

n = number of participants in the analysis set with observed data or non-responder imputation (NRI), excluding a total of seven participants with missing data due to medical or operational complications resulting from COVID-19.
*Primary endpoint: defined as total Mayo score ≤ 2 with no individual sub score >1.
^{†}Secondary endpoint: defined per Food and Drug Administration (FDA) definition with an mMS (endoscopic subscore = 0 or 1, ≥1 point decrease from baseline to achieve a stool frequency subscore = 0 or 1, and rectal bleeding subscore = 0).
^{‡}Defined as endoscopic subscore = 0 or 1.
^{§}Analyses were conducted for all dose groups; however, maintenance efficacy results are presented only for patients receiving the same dose during the induction and maintenance periods.
^{¶}As placebo was not administered during the maintenance period, no data for Week 56 are shown.
**For the endoscopic improvement endpoint at Week 56 with the 150 mg dose, data were available for n = 28 patients.

Statistically significant and clinically meaningful efficacy results were observed at every dose tested, both in the overall populations and in the biomarker-positive patient population. Results at the 450 mg dose at Week 14 are provided in Table 12, below. “Biologic-experienced” is defined as prior experience with an anti-TNF agent, an anti-integrin agent, and/or an anti-IL-12/23 agent.

TABLE 12
Summary of efficacy outcomes at 450 mg dose at Week 14

| | | Biomarker | Biologic- |
|---|---|---|---|
| | Overall | Positive | Experienced Biomarker |
| | Population at | Population at | Positive Population |
| | 450 mg Dose | 450 mg Dose | at 450 mg Dose |
| Clinical | 31% | 40% | 41% |
| Remission | | | |
| Endoscopic | 40% | 56% | 56% |
| Improvement | | | |

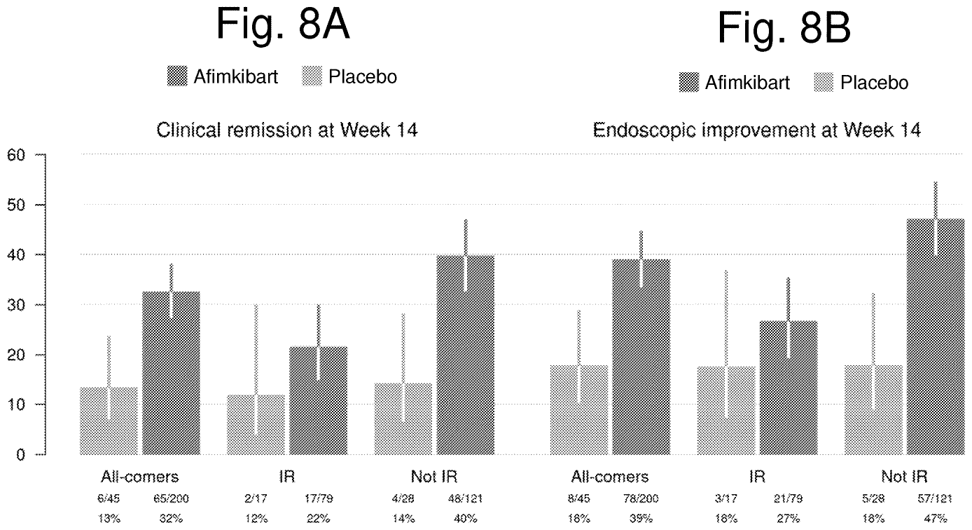

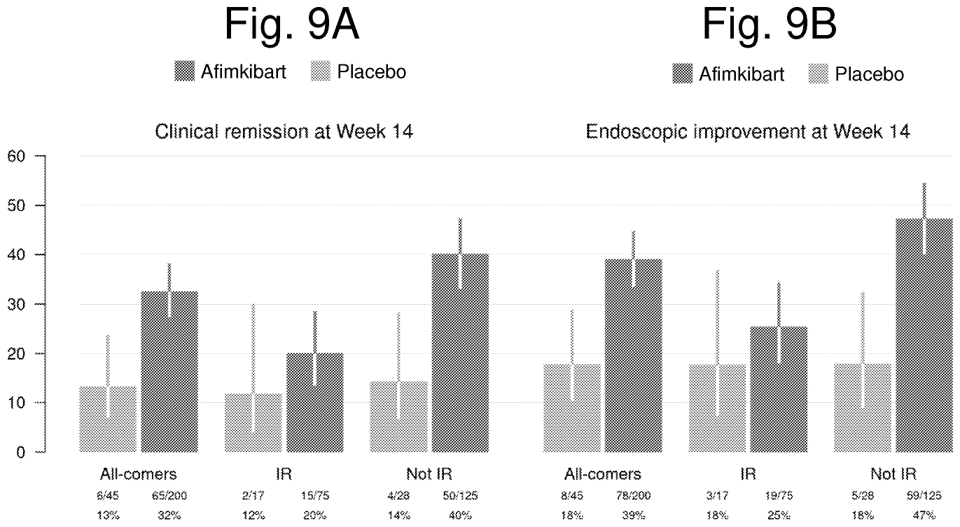

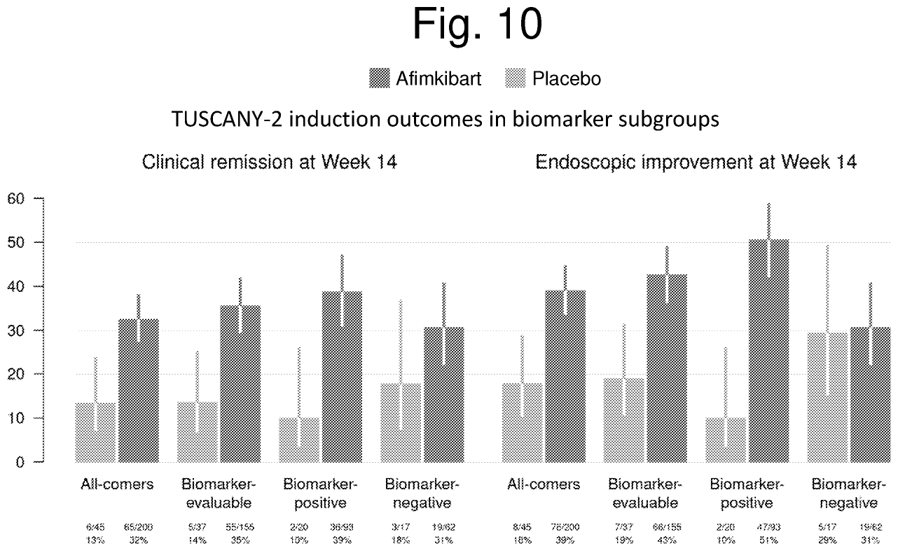

Efficacy results in subjects who were intolerant to, had an inadequate response to, or experienced a loss of response to a previously administered therapy are shown in FIGS. 8A, 8B, 8B, and 9B. FIGS. 8A and 8B show clinical remission and endoscopic improvement rates in patients who were intolerant to, had an inadequate response to, or experienced a loss of response to a prior advanced therapy (an anti-tumor necrosis factor (TNF) agent, anti-integrin agent, anti-IL-12/23 agent, and/or a Janus kinase (JAK) inhibitor). FIGS. 8A and 8B show clinical remission and endoscopic improvement rates in patients who were intolerant to, had an inadequate response to, or experienced a loss of response to a prior therapy comprising an anti-TNF agent.

### C. Safety

Afimkibart was well-tolerated, with no safety signals identified. No dose-related trends in adverse events were observed.

During the induction period, 47.8% (117/245) of patients reported ≥1 treatment-emergent adverse event (TEAE) (Tables 13 and 14). The most common TEAEs (occurring in ≥5% of patients) by preferred term were anemia (5.3%) and headache (5.3%) (Table 15). As is shown in Table 14, the treatment-emergent adverse events ‘infections,’ ‘anemia,’ and ‘injection site reactions’ were balanced across treatment arms. In total, 10 patients experienced serious TEAEs (placebo=4 patients; 50 mg=3 patients; 450 mg=3 patients) up to Week 14. Severe and serious adverse events were sporadic and were generally considered not to be related to the study drug. No patients discontinued the study due to TEAEs. A similar safety profile was observed during the maintenance period.

TABLE 13
Treatment-related adverse events observed in TUSCANY-2
in the safety analysis set in the induction period

| | | | RO7790121 | | RO7790121 | | RO7790121 | | | |
|---|---|---|---|---|---|---|---|---|---|---|
| | Placebo | | 50 mg | | 150 mg | | 450 mg | | Overall | |
| | (N = 45) | | (N = 47) | | (N = 62) | | (N = 91) | | (N = 245) | |
| Number of adverse events | 4 | | 15 | | 13 | | 21 | | 53 | |
| (AEs) | | | | | | | | | | |
| Participants with adverse | 4 | (8.9) | 6 | (12.8) | 9 | (14.5) | 13 | (14.3) | 32 | (13.1) |
| events, n (%) | | | | | | | | | | |
| Participants with severe | 1 | (2.2) | 1 | (2.1) | 0 | (0.0) | 1 | (1.1) | 3 | (1.2) |
| adverse events, n (%) | | | | | | | | | | |
| Participants with serious | 1 | (2.2) | 0 | (0.0) | 0 | (0.0) | 1 | (1.1) | 2 | (0.8) |
| adverse events, n (%) | | | | | | | | | | |
| Participants discontinued from | 0 | (0.0) | 0 | (0.0) | 0 | (0.0) | 0 | (0.0) | 0 | (0.0) |
| study due to adverse events^{†}, n | | | | | | | | | | |
| (%) | | | | | | | | | | |
| Participants discontinued study | 1 | (2.2) | 0 | (0.0) | 1 | (1.6) | 0 | (0.0) | 2 | (0.8) |
| drug due to adverse events and | | | | | | | | | | |
| continued study^{‡}, n (%) | | | | | | | | | | |
| Participants with dose reduced | 0 | (0.0) | 0 | (0.0) | 0 | (0.0) | 0 | (0.0) | 0 | (0.0) |

or temporary discontinuation
due to adverse events, n (%)

^{†}Participants who had an AE record that indicated that the AE caused the participant to be discontinued from the study.
^{‡}Participants who had an AE record that indicated that action taken with study treatment was drug withdrawn but AE did not cause the participant to be discontinued from study.
Adverse events were classified based on MedDRA version 25.1.
Abbreviations: mg = milligram.

TABLE 14
Treatment-related adverse events observed in TUSCANY-2 in the
safety analysis set in the induction period (pooled data)

| | Placebo | Pooled | |
|---|---|---|---|
| | (n = 45) | (n = 200) | 450 mg dose |
| Participants with | 56% | 45% | 53% |
| adverse events | | | |
| Participants with | 7% | 2% | 2% |
| severe adverse | | | |
| events | | | |
| Participants with | 7% | 4% | 3% |
| serious adverse | | | |
| events | | | |
| Participants | 0% | 0% | 0% |
| discontinued study | | | |
| due to adverse events | | | |
| Participants | 4% | 1% | 1% |
| discontinued study | | | |
| drug due to adverse | | | |
| events | | | |
| Participants with | 0% | 0% | 0% |
| dose reduced or | | | |
| temporary | | | |
| discontinuation due | | | |
| to adverse events | | | |
| Deaths | 0% | 0% | 0% |

Most Common Adverse Events/Adverse Events of Interest

| | | | |
|---|---|---|---|
| Infection and | 9% | 10% | 9% |
| infestations | | | |
| Anemia | 9% | 4% | 2% |
| Injection site reaction | 2% | 5% | 5% |
| COVID-19 | 2% | 1% | 1% |

TABLE 15
Treatment-related adverse events observed in ≥5% of participants
in the safety analysis set in the induction period

| | | | RO7790121 | | RO7790121 | | RO7790121 | | | |
|---|---|---|---|---|---|---|---|---|---|---|
| | Placebo | | 50 mg | | 150 mg | | 450 mg | | Overall | |
| | (N = 45) | | (N = 47) | | (N = 62) | | (N = 91) | | (N = 245) | |
| With any adverse event | 25 | (55.6) | 16 | (34.0) | 28 | (45.2) | 48 | (52.7) | 117 | (47.8) |
| Blood and lymphatic | 6 | (13.3) | 2 | (4.3) | 7 | (11.3) | 5 | (5.5) | 20 | (8.2) |
| system disorders | | | | | | | | | | |
| Anemia | 4 | (8.9) | 2 | (4.3) | 5 | (8.1) | 2 | (2.2) | 13 | (5.3) |
| Gastrointestinal disorders | 7 | (15.6) | 7 | (14.9) | 4 | (6.5) | 16 | (17.6) | 34 | (13.9) |
| Colitis ulcerative | 1 | (2.2) | 3 | (6.4) | 1 | (1.6) | 4 | (4.4) | 9 | (3.7) |
| Nausea | 1 | (2.2) | 3 | (6.4) | 2 | (3.2) | 2 | (2.2) | 8 | (3.3) |
| General disorders and | 3 | (6.7) | 1 | (2.1) | 6 | (9.7) | 11 | (12.1) | 21 | (8.6) |
| administration site | | | | | | | | | | |
| conditions | | | | | | | | | | |
| Fatigue | 0 | (0.0) | 0 | (0.0) | 1 | (1.6) | 5 | (5.5) | 6 | (2.4) |
| Pyrexia | 1 | (2.2) | 0 | (0.0) | 1 | (1.6) | 5 | (5.5) | 7 | (2.9) |
| Infections and infestations | 3 | (6.7) | 6 | (12.8) | 5 | (8.1) | 8 | (8.8) | 22 | (9.0) |
| Urinary tract infection | 0 | (0.0) | 3 | (6.4) | 0 | (0.0) | 2 | (2.2) | 5 | (2.0) |
| Nervous system disorders | 3 | (6.7) | 4 | (8.5) | 6 | (9.7) | 13 | (14.3) | 26 | (10.6) |
| Headache | 1 | (2.2) | 2 | (4.3) | 1 | (1.6) | 9 | (9.9) | 13 | (5.3) |

All values are n (%).
Adverse events were classified based on MedDRA version 25.1.

No impact of immunogenicity on clinical efficacy or safety results was observed. At Week 14 at the 450 mg dose, the anti-drug antibody (ADA) rate was 46%, and the neutralizing antibody (NAb) rate was 8%. These immunogenicity results are in line with approved biologics: Humira showed ADA rates of 32-46% and neutralizing antibody rates of 11-23% at Week 24 (Hanauer et al., 2021; Weinblatt et al., 2017, Cohen et al., 2019), and Skyrizi showed ADA rates of 19% and neutralizing antibody rates of 8% at Week 16 (Skyrizi (risankizumab) FDA Summary Basis of Approval). As is shown in Table 16, higher levels of ADAs were associated with lower doses of subcutaneous (SC) administration. The median times to first detection of ADAs and NAbs were 30-57 days and 58-85 days, respectively, during induction with the SC regimen.

TABLE 16
Immunogenicity by dose

| | 50 mg | 150 mg | 450 mg |
|---|---|---|---|
| | (N = 47) | (N = 62) | (N = 91) |
| Percent of subjects who were anti- | 91.1 | 64.8 | 39.8 |
| drug antibody positive (% ADA+) | | | |
| Percent of subjects who were | 33.3 | 17.9 | 7.9 |
| neutralizing antibody positive | | | |
| (% nAb) | | | |
| Median time to first ADA (days) | 30 | 57 | 56 |
| Median time to nAb (days) | 58 | 84 | 85 |

### C. Induction and Maintenance Exposure-Response

As is shown in Table 17, higher exposures to afimkibart (by 14-week Cₐᵥₑ tertiles) in the induction phase were associated with higher Week 14 outcome rates. 14-week Cₐᵥₑ is the predicted average concentration of afimkibart up to Week 14.

TABLE 17
Exposure response by 14-week Cₐᵥₑ tertiles

| | | | | Difference of 3^{rd} | Difference of 3^{rd} |
|---|---|---|---|---|---|
| | 1^{st} | 2^{nd} | 3^{rd} | Tertile from 1^{st} | Tertile from 2^{nd} |
| | Tertile | Tertile | Tertile | Tertile | Tertile |
| mMS remission (Week 14 | 26.2% | 32.3% | 38.5% | 12.3%; 1.5-fold | 6.2%; 1.2-fold |
| ES = 0 or 1, SFS = 0 or 1 | | | | | |
| and lower than baseline, | | | | | |
| RBS = 0) | | | | | |
| Endoscopic improvement | 33.8% | 38.5% | 47.4% | 13.6%; 1.4-fold | 8.9%; 1.2-fold |
| mMS remission (biologic- | 14.8% | 22.2% | 32.1% | 17.3%; 2.2-fold | 9.9%; 1.4-fold |
| experienced) | | | | | |
| Endoscopic improvement | 25.9% | 22.2% | 39.3% | 13.4%; 1.5-fold | 17.1%; 1.8-fold |
| (biologic-experienced) | | | | | |

As is shown in Table 18, higher exposures to afimkibart (by 56-week Cₐᵥₑ tertiles) were associated with higher Week 56 outcome rates in induction responders who continued on the same dose in the maintenance phase. 56-week Cₐᵥₑ is the predicted average concentration of afimkibart up to Week 56.

TABLE 18
Exposure response by 56-week Cₐᵥₑ tertiles

| | | | | Absolute | Absolute |
|---|---|---|---|---|---|
| | | | | difference: | difference: |
| | 1^{st} Tertile | 2^{nd} Tertile | 3^{rd} Tertile | 3^{rd} vs. 1^{st} | 3^{rd} vs. 2^{nd} |
| | n/N2 (%) | n/N2 (%) | n/N2 (%) | Tertile | Tertile |
| mMS remission (Week | 9/32 | 11/32 | 13/32 | 12.5% | 6.2% |
| 56 ES = 0 or 1, SFS = 0 | (28.1%) | (34.4%) | (40.6%) | | |
| or 1 and lower than | | | | | |
| baseline, RBS = 0) | | | | | |
| Endoscopic | 13/33 | 11/32 | 17/33 | 12.1% | 17.1% |
| improvement | (39.4%) | (34.4%) | (51.5%) | | |

As is shown in Table 19, almost all of the patients that received the 450 mg dose (about 74% of patients in the induction phase, and about 96% of patients in the maintenance phase) had exposures in the highest tertile, indicating that this dose was necessary in order to achieve systemic exposures that corresponded to maximal and sustained efficacy.

TABLE 19
Pharmacokinetic exposure during induction and maintenance

| | | 1^{st} Tertile | | 2^{nd} Tertile | | 3^{rd} Tertile | |
|---|---|---|---|---|---|---|---|
| Induction C_{ave, 14 wk} | 50 mg (n = 47) | 47 | (100%) | 0 | (0%) | 0 | (0%) |
| number of patients | 150 mg (n = 60) | 17 | (28.3%) | 43 | (71.7%) | 0 | (0%) |
| and (%) by dose | 450 mg (n = 88) | 1 | (1.1%) | 22 | (25%) | 65 | (73.9%) |
| Sample size was n = | | | | | | | |
| 65 each in 1st, 2nd | | | | | | | |
| and 3rd tertile. | | | | | | | |
| Maintenance² C_{ave, 56 wk} | 50 mg → 50 mg | 30 | (71.4%) | 12 | (28.6%) | 0 | (0%) |
| number of patients | (n = 42) | | | | | | |
| and (%) by dose | 150 mg → 150 mg | 2 | (7.7%) | 19 | (73.1%) | 5 | (19.2%) |
| Sample size was n = | (n = 26) | | | | | | |
| 32 each in 1st, 2nd | 450 mg → 450 mg | 0 | (0%) | 1 | (3.6%) | 27 | (96.4%) |
| and 3rd tertile. | (n = 28) | | | | | | |

### D. The 450 mg Dose

The 450 mg SC Q4W dosing regimen was the only dose arm that trended to increasing efficacy over time.

At the 450 mg dose, superior efficacy was observed at Week 52 vs. Week 14 (all comers 36% vs 29%; biomarker-positive patients 43% vs 33%).

The percent of patients having a sustained clinical response (when receiving the same dose in the induction phase and the maintenance phase) was highest at the 450 mg dose (75%) (i.e., higher efficacy was observed in participants that maintained the same 450 mg dose during the induction and maintenance phases as compared to participants that received lower doses (50 mg or 150 mg).

Higher efficacy was observed in participants that maintained the same 450 mg dose during maintenance phase as compared to patients who received lower doses (e.g. 50 mg or 150 mg).

As is shown above, an exposure-response analysis with individual exposures suggested that participants with the highest tertile average PK concentration over 56 weeks had numerically higher modified remission (mMS remission), endoscopic improvement, sustained modified remission, and sustained endoscopic improvement response than the lower two tertiles. Almost all of the patients that received the 450 mg dose in the maintenance phase (about 96%) had exposures in the highest tertile, indicating that this dose was necessary in order to achieve systemic exposures that corresponded to maximal and sustained efficacy over the long term.

Further, during the maintenance phase of the study, the anti-drug antibody (ADA) and neutralizing antibody (NAb) persistent response rates in the 450 mg→450 mg treatment sequence arm (450 mg dose administered in the induction and maintenance phases) were the lowest as compared to other treatment sequence rates (see Table 16). Minimal to no impact of ADA status on serum afimkibart was observed in the 450 mg→450 mg sequence.

### E. Conclusions

Data from the Phase 2b TUSCANY-2 dose-ranging study demonstrate that afimkibart has a positive benefit/risk profile, with clinically meaningful improvements in patients with moderately to severely active UC.

What is claimed is:

1. A method of determining the risk of a patient being non-responsive to a therapeutic dose of an anti-TNF-like ligand 1A (TL1A) antibody comprising:
performing a genotyping assay on a biological sample from the patient to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and
determining the risk of being non-responsive to the therapeutic dose of an anti-TL1A antibody, wherein the risk is higher in a haplotype B carrier patient than in a haplotype B non-carrier patient.

2. The method of claim 1, wherein the patient has inflammatory bowel disease (IBD).

3. A method for treating inflammatory bowel disease (IBD) in a patient, the method comprising:
(a) performing a genotyping assay to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and
(b) administering to the patient an effective amount of an anti-TL1A antibody, wherein the patient has been determined to be a haplotype B non-carrier.

4. The method of claim 3, wherein the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses.

5. The method of claim 3, wherein the anti-TL1A antibody is administered to the patient in a maintenance dosing regimen after completion of an induction dosing regimen, said maintenance dosing regimen comprising a plurality of individual maintenance doses separated from each other by at least 2 weeks.

6. A method for treating IBD in a patient, the method comprising:
(a) performing a genotyping assay to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient;
(b) administering to the patient an anti-TL1A antibody in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and
(c) administering to the patient a subsequent maintenance dosing regimen after completion of the induction dosing regimen, said maintenance dosing regimen comprising a plurality of individual maintenance doses separated from each other by at least 2 weeks.

7. The method of any one of claims 2-6, wherein the IBD is ulcerative colitis (UC).

8. The method of claim 7, wherein the UC is moderate to severe UC.

9. The method of any one of claims 2-6, wherein the IBD is Crohn's disease (CD).

10. The method of any one of claims 1-9, wherein the at least two SNPs are SNP rs3810936 and SNP rs7869487.

11. The method of claim 10, wherein the genotyping assay comprises determining the presence of only two SNPs in the biological sample from the patient, wherein the two SNPs are SNP rs3810936 and SNP rs7869487.

12. The method of claim 10 or 11, wherein the patient is determined to be a haplotype B non-carrier by any one of the following biomarker statuses in the biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487.

13. The method of claim 10, wherein (a) the genotyping assay comprises determining the presence of SNP rs3810936, SNP rs7869487, and SNP rs7848647 in the biological sample from the patient; or (b) the genotyping assay comprises determining the presence of SNP rs3810936, SNP rs7869487, and SNP rs6478109 in the biological sample from the patient.

14. The method of claim 10, wherein the genotyping assay comprises determining the presence of SNP rs3810936, SNP rs7869487, SNP rs6478108, and SNP rs6478109 in the biological sample from the patient.

15. The method of any one of claims 1-14, wherein the genotyping assay comprises qPCR.

16. The method of any one of claims 1-15, wherein the genotyping assay comprises sequencing.

17. The method of any one of claims 1-16, wherein the genotyping assay comprises long sequencing.

18. The method of any one of claims 1-17, wherein the genotyping assay is performed on a microarray.

19. The method of any one of claims 1-18, wherein the genotyping assay is a fluorescence-based assay.

20. The method of any one of claims 1-19, wherein the biological sample is blood.

21. The method of any one of claims 1-19, wherein the biological sample is buccal cells.

22. A method of determining the risk of a patient being non-responsive to a therapeutic dose of an anti-TL1A antibody, wherein the patient has inflammatory bowel disease (IBD), the method comprising:
(a) performing a genotyping assay on a biological sample from the patient to determine whether the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487; and
(b) identifying the patient as having a high risk of being non-responsive to a therapeutic dose of an anti-TL1A antibody if the patient is a haplotype B carrier; or
(c) identifying the patient as having a low risk of being non-responsive to a therapeutic dose of an anti-TL1A antibody if the patient is a haplotype B non-carrier.

23. The method of claim 22, wherein the patient has been determined to be a haplotype B non-carrier for TNFSF15, and the method further comprises administering to the patient an effective amount of an anti-TL1A antibody.

24. The method of claim 23, wherein the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses.

25. The method of claim 23, wherein the anti-TL1A antibody is administered to the patient in a maintenance dosing regimen after completion of an induction dosing regimen, said maintenance dosing regimen comprising a plurality of individual maintenance doses separated from each other by at least 2 weeks.

26. A method for treating IBD in a patient, the method comprising:
(a) performing a genotyping assay on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487; and
(b) administering to the patient an effective amount of an anti-TL1A antibody wherein the patient has been determined to be a haplotype B non-carrier.

27. The method of claim 26, wherein the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses.

28. The method of claim 26, wherein the anti-TL1A antibody is administered to the patient in a subsequent maintenance dosing regimen after completion of an induction dosing regimen, said maintenance dosing regimen comprising a plurality of individual maintenance doses separated from each other by at least 2 weeks.

29. A method for treating IBD in a patient, the method comprising administering to the patient an effective amount of an anti-TL1A antibody;
wherein the patient has been determined to be a haplotype B non-carrier for TNFSF15 by any one of the following biomarker statuses in a biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487.

30. The method of claim 29, wherein the anti-TL1A antibody is administered to the patient in an induction dosing regimen sufficient to improve signs and symptoms of IBD by at least 12 weeks after the start of treatment with the anti-TL1A antibody, said induction dosing regimen comprising a plurality of individual induction doses.

31. The method of claim 29, wherein the anti-TL1A antibody is administered to the patient in a subsequent maintenance dosing regimen after completion of an induction dosing regimen, said maintenance dosing regimen comprising a plurality of individual maintenance doses separated from each other by at least 2 weeks.

32. The method of any one of claims 4, 6-21, 24, 27, and 30, wherein the individual induction doses are separated from each other by at least 1 week, least 2 weeks, at least 3 weeks, or at least 4 weeks.

33. The method of any one of claims 4, 6-21, 24, 27, 30, and 32, wherein the individual induction doses are separated from each other by at least 1 month.

34. The method of claim 32, wherein the individual induction doses are administered 4 weeks apart.

35. The method of any one of claims 4, 6-21, 24, 27, 30, and 32-34, wherein the individual induction dose is administered intravenously or subcutaneously.

36. The method of claim 35, wherein each of the individual induction doses is administered subcutaneously.

37. The method of any one of claims 4, 6-21, 24, 27, 30, and 32-36, wherein the individual induction dose is administered at 50, 150 or 450 mg.

38. The method of claim 37, wherein each of the individual induction doses is administered at a dose of 50 mg.

39. The method of claim 37, wherein each of the individual induction doses is administered at a dose of 150 mg.

40. The method of claim 37, wherein each of the individual induction doses is administered at a dose of 450 mg.

41. The method of any one of claims 4-21 and 24-40, wherein the induction dosing regimen comprises four individual induction doses.

42. The method of any one of claims 4-21, 24, 25, 27, 28, and 30-41, wherein the induction dosing regimen comprises four individual induction doses administered 4 weeks apart, and wherein each of the individual induction doses is administered subcutaneously at a dose of 50, 150, or 450 mg.

43. The method of any one of claims 5-21, 25, 28, 31, 41, and 42, wherein the individual maintenance doses are administered at least 2 weeks, at least 3 weeks, or at least 4 weeks apart.

44. The method of any one of claims 5-21, 25, 28, 31, and 41-43, wherein the individual maintenance doses are administered at least 1 month apart.

45. The method of claim 43, wherein the individual maintenance doses are administered 4 weeks apart.

46. The method of any one of claims 5-21, 25, 28, and 41-45, wherein the individual maintenance dose is administered intravenously or subcutaneously.

47. The method of claim 46, wherein each of the individual maintenance doses is administered subcutaneously.

48. The method of any one of claims 5-21, 25, 28, and 41-47, wherein the individual maintenance dose is administered at 50, 150, or 450 mg.

49. The method of claim 48, wherein each of the individual maintenance doses is administered at a dose of 50 mg.

50. The method of claim 48, wherein each of the individual maintenance doses is administered at a dose of 150 mg.

51. The method of claim 48, wherein each of the individual maintenance doses is administered at a dose of 450 mg.

52. The method of any one of claims 5-21, 25, 28, and 41-51, wherein the maintenance dosing regimen comprises at least ten individual maintenance doses.

53. The method of any one of claims 5-21, 25, 28, and 41-52, wherein the induction dosing regimen comprises ten individual maintenance doses administered 4 weeks apart, and wherein each of the individual maintenance doses is administered subcutaneously at a dose of 50, 150, or 450 mg.

54. The method of claim 53, wherein each of the individual maintenance doses is administered subcutaneously at a dose of 450 mg.

55. The method of any one of claims 3-54, further comprising:
(a) determining the expression level of one or more candidate genes in a biological sample from the patient, and
(b) identifying that the biological sample contains an abnormal expression level of the one of more candidate genes.

56. The method of claim 55, wherein the one or more candidate genes is selected from the group consisting of SOWAHB, COLCA2, TBX20, FRZB, HOXB5, NET1, FOXD2, DESI1, PARK2, PKDREJ, IL-1 B, IL-23A, IFNG, IL-12RB1, IL-21 R, IRF4, BATF, CD80/86, HLA-DRB5/DQB1/DRB1, HLA-DRA, CD40, ICOS, MMP3, MMP7, MMP10, and CHI3L.

57. The method of claim 56, wherein the one or more candidate genes are selected from the group consisting of SOWAHB, COLCA2, TBX20, FRZB, HOXB5, NET1, FOXD2, DESI1, PARK2, and PKDREJ.

58. The method of any one of claims 55-57, wherein the expression level of the one or more candidate genes is compared against a baseline expression level which is a) based on the expression level of the one or more candidate genes for a healthy individual who is not suffering from IBD or UC; or b) based on an estimated expression level for individuals who are non-responsive to anti-TL1A antibody treatment.

59. The method of claim 58, wherein the abnormal expression level of the one or more candidate genes is at least 50% greater or lesser from the baseline level.

60. The method of any one of claims 3-59, further comprising:
a) determining the expression level of one or more candidate bacterial strains in a stool sample from the patient, and
b) identifying that the sample contains an increased or decreased level of the one of more candidate bacterial strains.

61. The method of claim 60, wherein the candidate bacterial strain level is increased, and the candidate bacterial strain is selected from the group consisting of Streptococcus salivarius, Streptococcus parasanguinis, and Haemophilus parainfluenzae.

62. The method of claim 61, wherein the candidate bacterial strain level is decreased, and the candidate bacterial strain is selected from the group consisting of Ruminococcus albus, Ruminococcus callidus, Ruminococcus bromii, Ruminococcus gnavus, and Bifidobacterium bifidum.

63. The method of any one of claims 1-62, wherein the anti-TL1A antibody comprises three CDRs from the variable heavy chain region having the sequence shown in SEQ ID NO: 1 and three CDRs from the variable light chain region having the sequence shown in SEQ ID NO: 2.

64. The method of any one of claims 1-62, wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

65. The method of any one of claims 1-64, wherein the anti-TL1A antibody comprises a variable heavy chain region having the sequence shown in SEQ ID NO: 1 and a variable light chain region having the sequence shown in SEQ ID NO: 2.

66. The method of any one of claims 1-65, wherein the anti-TL1A antibody comprises a heavy chain having the sequence shown in SEQ ID NO: 9 and a light chain having the sequence shown in SEQ ID NO: 10.

67. The method of any one of claims 1-66, wherein the anti-TL1A antibody is afimkibart.

68. A method for treating inflammatory bowel disease (IBD) in a patient, the method comprising:
(a) performing a genotyping assay to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and
(b) administering to the patient an effective amount of an anti-TL1A antibody, wherein the patient has been determined to be a haplotype B non-carrier;
wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

69. The method of claim 68, wherein the at least two SNPs are SNP rs3810936 and SNP rs7869487.

70. The method of claim 69, wherein the assay comprises determining the presence of only two SNPs in the biological sample from the patient, and wherein the two SNPs are SNP rs3810936 and SNP rs7869487.

71. The method of any one of claims 1-62, wherein the anti-TL1A antibody comprises sequence pairs selected from the group consisting of SEQ ID NOs: 2 and 11; SEQ ID NOs: 2 and 12; SEQ ID NOs: 2 and 13; SEQ ID NOs: 2 and 14; SEQ ID NOs: 2 and 15; SEQ ID NOs: 2 and 16; SEQ ID NOs: 2 and 17; SEQ ID NOs: 2 and 18; SEQ ID NOs: 2 and 19; SEQ ID NOs: 20 and 24; SEQ ID NOs: 21 and 25; SEQ ID NOs: 22 and 26; SEQ ID NOs: 23 and 27; SEQ ID NOs: 28 and 29; SEQ ID NOs: 30 and 31; SEQ ID NOs: 32 and 33; SEQ ID NOs: 35 and 44; SEQ ID NOs: 53 and 54; SEQ ID NOs: 61 and 62; SEQ ID NOs: 63 and 64; SEQ ID NOs: 65 and 64; SEQ ID NOs: 66 and 64; and SEQ ID NOs: 67 and 64.

72. The method of any one of claims 1-62, wherein the anti-TL1A antibody comprises three CDRs from the variable heavy chain region having the sequence shown in SEQ ID NO: 53 and three CDRs from the variable light chain region having the sequence shown in SEQ ID NO: 54.

73. The method of any one of claims 1-62, wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50.

74. The method of any one of claims 1-62, 72, and 73, wherein the anti-TL1A antibody comprises a variable heavy chain region having the sequence shown in SEQ ID NO: 53 and a variable light chain region having the sequence shown in SEQ ID NO: 54.

75. The method of any one of claims 1-62 and 72-74, wherein the anti-TL1A antibody comprises a heavy chain having the sequence shown in SEQ ID NO: 35 and a light chain having the sequence shown in SEQ ID NO: 44.

76. The method of any one of claims 1-62 and 72-75, wherein the anti-TL1A antibody is tulisokibart.

77. A method for treating inflammatory bowel disease (IBD) in a patient, the method comprising:
(a) performing a genotyping assay to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and
(b) administering to the patient an anti-TL1A antibody, wherein the patient has been determined to be a haplotype B non-carrier;
wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50.

78. The method of claim 77, wherein the at least two SNPs are SNP rs3810936 and SNP rs7869487.

79. The method of claim 78, wherein the assay comprises determining the presence of only two SNPs in the biological sample from the patient, wherein the two SNPs are SNP rs3810936 and SNP rs7869487.

80. The method of any one of claims 3-79, further comprising treatment with an IL-23 antagonist.

81. The method of any one of claims 22-31, 68-70, and 77-79, wherein the IBD is ulcerative colitis (UC).

82. The method of claim 81, wherein the UC is moderate to severe UC.

83. The method of any one of claims 22-31, 68-70, and 77-79, wherein the IBD is Crohn's disease (CD).

84. The method of any one of claims 1-83, wherein the patient is a human.

85. A method of treating an inflammatory bowel disease (IBD) in a patient, the method comprising administering to the patient an effective amount of an anti-TNF-like ligand 1A (TL1A) antibody in a dosing regimen comprising an induction dosing regimen and a subsequent maintenance dosing regimen;
wherein the induction dosing regimen comprises a plurality of individual induction doses and the maintenance dosing regimen comprising a plurality of individual maintenance doses;
wherein the anti-TL1A antibody is administered subcutaneously in the maintenance phase, and wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

86. The method of claim 85, wherein the individual induction doses are separated from each other by at least 1 week, least 2 weeks, at least 3 weeks, or at least 4 weeks.

87. The method of claim 85 or 86, wherein the individual induction doses are separated from each other by at least 1 month.

88. The method of claim 86, wherein the individual induction doses are administered 4 weeks apart.

89. The method of any one of claims 85-88, wherein the individual induction dose is administered intravenously or subcutaneously.

90. The method of claim 89, wherein each of the individual induction doses is administered subcutaneously.

91. The method of any one of claims 85-90, wherein the individual induction dose is administered at 50, 150, or 450 mg.

92. The method of claim 91, wherein each of the individual induction doses is administered at a dose of 50 mg.

93. The method of claim 91, wherein each of the individual induction doses is administered at a dose of 150 mg.

94. The method of claim 91, wherein each of the individual induction doses is administered at a dose of 450 mg.

95. The method of any one of claims 85-94, wherein the induction dosing regimen comprises four individual induction doses.

96. The method of any one of claims 85-95, wherein the induction dosing regimen comprises four individual induction doses administered 4 weeks apart, and wherein each of the individual induction doses is administered subcutaneously at a dose of 50, 150, or 450 mg.

97. The method of any one of claims 85-96, wherein the individual maintenance doses are administered at least 2 weeks, at least 3 weeks, or at least 4 weeks apart.

98. The method of any one of claims 85-97, wherein the individual maintenance doses are administered at least 1 month apart.

99. The method of claim 97, wherein the individual maintenance doses are administered 4 weeks apart.

100. The method of any one of claims 85-99, wherein the individual maintenance dose is administered at 50, 150, or 450 mg.

101. The method of claim 100, wherein each of the individual maintenance doses is administered at a dose of 50 mg.

102. The method of claim 100, wherein each of the individual maintenance doses is administered at a dose of 150 mg.

103. The method of claim 100, wherein each of the individual maintenance doses is administered at a dose of 450 mg.

104. The method of any one of claims 85-103, wherein the maintenance dosing regimen comprises at least ten individual maintenance doses.

105. The method of any one of claims 85-104, wherein the induction dosing regimen comprises ten individual maintenance doses administered 4 weeks apart, wherein each of the individual maintenance doses is administered subcutaneously at a dose of 50, 150, or 450 mg.

106. The method of claim 105, wherein each of the individual maintenance doses is administered subcutaneously at a dose of 450 mg.

107. A method of treating an inflammatory bowel disease (IBD) in a patient, the method comprising administering to the patient an effective amount of an anti-TNF-like ligand 1A (TL1A) antibody in a dosing regimen comprising an induction dosing regimen and a subsequent maintenance dosing regimen; wherein:
(a) the induction dosing regimen comprises subcutaneous administration of four individual induction doses of the anti-TL1A antibody at a dose of about 50 mg, 150 mg, or 450 mg, wherein the individual induction doses are administered 4 weeks apart; and
(b) the maintenance dosing regimen comprises subcutaneous administration of a plurality of individual maintenance doses of the anti-TL1A antibody at a dose of about 50 mg, 150 mg, or 450 mg, wherein the individual maintenance doses are administered 4 weeks apart; and
wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

108. A method of treating an inflammatory bowel disease (IBD) in a patient, the method comprising administering to the patient an effective amount of an anti-TNF-like ligand 1A (TL1A) antibody in a dosing regimen comprising an induction dosing regimen and a subsequent maintenance dosing regimen; wherein:
(a) the induction dosing regimen comprises a plurality of individual induction doses, wherein the individual induction doses are separated from each other by at least 1 week, least 2 weeks, at least 3 weeks, or at least 4 weeks; and
(b) the maintenance dosing regimen comprises subcutaneous administration of a plurality of individual maintenance doses of the anti-TL1A antibody at a dose of about 450 mg, wherein the individual maintenance doses are administered 4 weeks apart; and
wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

109. The method of claim 108, wherein the induction dosing regimen comprises subcutaneous administration of four individual induction doses of the anti-TL1A antibody at a dose of about 450 mg, wherein the individual induction doses are administered 4 weeks apart.

110. The method of any one of claims 85-109, wherein the first dose of the maintenance phase is administered about two weeks after administration of the final dose of the induction phase.

111. The method of any one of claims 85-110, wherein the dosing regimen has a duration of about 52 weeks.

112. The method of any one of claims 85-111, wherein the anti-TL1A antibody comprises a variable heavy chain region having the sequence shown in SEQ ID NO: 1 and a variable light chain region having the sequence shown in SEQ ID NO: 2.

113. The method of any one of claims 85-112, wherein the anti-TL1A antibody comprises a heavy chain having the sequence shown in SEQ ID NO: 9 and a light chain having the sequence shown in SEQ ID NO: 10.

114. The method of any one of claims 85-113, wherein the anti-TL1A antibody is afimkibart.

115. The method of any one of claims 85-114, wherein the patient has been determined to be a haplotype B non-carrier for TNFSF15.

116. The method of any one of claims 85-115, wherein the IBD is ulcerative colitis (UC).

117. The method of claim 116, wherein the UC is moderate to severe UC.

118. The method of any one of claims 85-117, wherein the patient is a human.

119. The method of any one of claims 85-118, wherein, in a population of patients treated according to the method, the treating results in an increase in the proportion of patients who have achieved clinical remission at the end of the induction phase as compared to a reference population.

120. The method of claim 119, wherein the induction phase has a duration of about 14 weeks, and the treating results in an increase in the proportion of patients who have achieved clinical remission at Week 14.

121. The method of claim 120, wherein at least about 15% of patients in the population of patients have achieved clinical remission at Week 14.

122. The method of claim 121, wherein at least about 40% of patients in the population of patients have achieved clinical remission at Week 14.

123. The method of any one of claims 85-122, wherein, in a population of patients treated according to the method, the treating results in an increase in the proportion of patients who have achieved clinical remission at the end of the maintenance phase as compared to a reference population.

124. The method of claim 123, wherein the dosing regimen has a duration of about 56 weeks, and the treating results in an increase in the proportion of patients who have achieved clinical remission at Week 56.

125. The method of claim 124, wherein at least about 20% of patients in the population of patients have achieved clinical remission at Week 56.

126. The method of claim 125, wherein at least about 55% of patients in the population of patients have achieved clinical remission at Week 56.

127. The method of any one of claims 119-126, wherein clinical remission is a total Mayo Score of ≤2, with no individual subscore >1.

128. The method of any one of claims 119-126, wherein clinical remission is a modified Mayo Score (mMS)≤2 with stool frequency subscore (SFS)=0 or 1, rectal bleeding subscore (RBS)=0, and endoscopic subscore (ES)=0 or 1.

129. The method of any one of claims 85-128, wherein, in a population of patients treated according to the method, the treating results in an increase in the proportion of patients who have achieved endoscopic improvement at the end of the induction phase as compared to a reference population.

130. The method of claim 129, wherein the induction phase has a duration of about 14 weeks, and the treating results in an increase in the proportion of patients who have achieved endoscopic improvement at Week 14.

131. The method of claim 130, wherein at least about 25% of patients in the population of patients have achieved endoscopic improvement at Week 14.

132. The method of claim 131, wherein at least about 50% of patients in the population of patients have achieved endoscopic improvement at Week 14.

133. The method of any one of claims 85-132, wherein, in a population of patients treated according to the method, the treating results in an increase in the proportion of patients who have achieved endoscopic improvement at the end of the maintenance phase as compared to a reference population.

134. The method of claim 133, wherein the dosing regimen has a duration of about 56 weeks, and the treating results in an increase in the proportion of patients who have achieved endoscopic improvement at Week 56.

135. The method of claim 134, wherein at least about 20% of patients in the population of patients have achieved endoscopic improvement at Week 56.

136. The method of claim 135, wherein at least about 65% of patients in the population of patients have achieved endoscopic improvement at Week 56.

137. The method of any one of claims 129-136, wherein clinical remission is an endoscopic subscore of 0 or 1.

138. The method of any one of claims 85-137, wherein, in a population of patients treated according to the method, the treating results in an increase in the proportion of patients who have achieved endoscopic remission, a clinical response, symptomatic remission, or deep remission at the end of the induction phase as compared to a reference population.

139. The method of any one of claims 85-138, wherein, in a population of patients treated according to the method, the treating results in an increase in the proportion of patients who have achieved endoscopic remission, a clinical response, symptomatic remission, or deep remission at the end of the maintenance phase as compared to a reference population.

140. The method of any one of claims 119-139, wherein the reference population is a population of patients who have not been treated with an anti-TL1A antibody.

141. The method of claim 140, wherein the reference population is a population of patients who have been treated with a placebo.

142. A kit comprising an anti-TNF-like ligand 1A (TL1A) antibody and a package insert comprising instructions for using the antibody for treating an inflammatory bowel disease (IBD) in a patient in need thereof according to the method of any one of claims 1-141.

143. An anti-TL1A antibody for use in treating IBD in a patient, wherein:
(a) a genotyping assay is to be performed to determine if the patient is a haplotype B carrier or a haplotype B non-carrier for TNFSF15, the assay comprising determining the presence of at least two single nucleotide polymorphisms (SNPs) selected from rs3810936, rs6478108, rs6478109, rs7848647, and rs7869487 in a biological sample from the patient; and
(b) an effective amount of the anti-TL1A antibody is to be administered to the patient, wherein the patient has been determined to be a haplotype B non-carrier.

144. An anti-TL1A antibody for use in treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient;
(b) the anti-TL1A antibody is to be administered to the patient in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and
(c) the anti-TL1A antibody is to be administered to the patient in a subsequent maintenance dosing regimen after completion of the induction dosing regimen, said maintenance dosing regimen comprising a plurality of individual maintenance doses separated from each other by at least 2 weeks.

145. An anti-TL1A antibody for use in treating IBD in a patient, wherein:
(a) a genotyping assay is to be performed on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487; and
(b) an effective amount of the anti-TL1A antibody is to be administered to the patient, wherein the patient has been determined to be a haplotype B non-carrier.

146. An anti-TL1A antibody for use in treating IBD in a patient, wherein an effective amount of the anti-TL1A antibody is to be administered to the patient;
wherein the patient has been determined to be a haplotype B non-carrier for TNFSF15 by any one of the following biomarker statuses in a biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487.

147. An anti-TL1A antibody for use in treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and
(b) an effective amount of the anti-TL1A antibody is to be administered to the patient, wherein the patient has been determined to be a haplotype B non-carrier;
wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

148. An anti-TL1A antibody for use in treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and
(b) the anti-TL1A antibody is to be administered to the patient, wherein the patient has been determined to be a haplotype B non-carrier;
wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50.

149. An anti-TL1A antibody for use in treating an IBD in a patient, wherein an effective amount of the anti-TL1A antibody is to be administered to the subject in a dosing regimen comprising an induction dosing regimen and a subsequent maintenance dosing regimen;
wherein the induction dosing regimen comprises a plurality of individual induction doses and the maintenance dosing regimen comprising a plurality of individual maintenance doses;
wherein the anti-TL1A antibody is administered subcutaneously in the maintenance phase, and wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

150. An anti-TL1A antibody for use in treating an IBD in a patient, wherein an effective amount of the anti-TL1A antibody is to be administered to the subject in a dosing regimen comprising an induction dosing regimen and a subsequent maintenance dosing regimen, wherein:
(a) the induction dosing regimen comprises subcutaneous administration of four individual induction doses of the anti-TL1A antibody at a dose of about 50 mg, 150 mg, or 450 mg, wherein the individual induction doses are administered 4 weeks apart; and
(b) the maintenance dosing regimen comprises subcutaneous administration of a plurality of individual maintenance doses of the anti-TL1A antibody at a dose of about 50 mg, 150 mg, or 450 mg, wherein the individual maintenance doses are administered 4 weeks apart, and
wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

151. An anti-TL1A antibody for use in treating an inflammatory bowel disease (IBD) in a patient, wherein an effective amount of the anti-TL1A antibody is to be administered to the subject in a dosing regimen comprising an induction dosing regimen and a subsequent maintenance dosing regimen, wherein:
(a) the induction dosing regimen comprises a plurality of individual induction doses, wherein the individual induction doses are separated from each other by at least 1 week, least 2 weeks, at least 3 weeks, or at least 4 weeks; and
(b) the maintenance dosing regimen comprises subcutaneous administration of a plurality of individual maintenance doses of the anti-TL1A antibody at a dose of about 450 mg, wherein the individual maintenance doses are administered 4 weeks apart, and
wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

152. Use of an anti-TL1A antibody in the manufacture of a medicament for treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and
(b) an effective amount of the anti-TL1A antibody is to be administered to the patient, wherein the patient has been determined to be a haplotype B non-carrier.

153. Use of an anti-TL1A antibody in the manufacture of a medicament for treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient;
(b) the anti-TL1A antibody is to be administered to the patient in an induction dosing regimen sufficient to improve signs and symptoms of IBD by at least 12 weeks after the start of treatment with the anti-TL1A antibody, said induction dosing regimen comprising a plurality of individual induction doses, wherein the patient has been determined to be a haplotype B non-carrier; and
(c) the anti-TL1A antibody is to be administered to the patient in a subsequent maintenance dosing regimen after completion of the induction dosing regimen, said maintenance dosing regimen comprising a plurality of individual maintenance doses separated from each other by at least 2 weeks.

154. Use of an anti-TL1A antibody in the manufacture of a medicament for treating IBD in a patient, wherein:
(a) a genotyping assay is to be performed on a biological sample from the patient to determine if the patient is a haplotype B carrier or a haplotype B non-carrier for TNFSF15, wherein the assay comprises determining the presence of SNP rs3810936 and SNP rs7869487 in a biological sample from the patient, and wherein a haplotype B non-carrier for TNFSF15 is identified by any one of the following biomarker statuses in the biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487; and
(b) an effective amount of the anti-TL1A antibody is to be administered to the patient, wherein the patient has been determined to be a haplotype B non-carrier.

155. Use of an anti-TL1A antibody in the manufacture of a medicament for treating IBD in a patient, wherein an effective amount of the anti-TL1A antibody is to be administered to the patient, and
wherein the patient has been determined to be a haplotype B non-carrier for TNFSF15 by any one of the following biomarker statuses in a biological sample from the patient:
(i) the genotype of the sample is homozygous for the reference allele at rs3810936 and is homozygous for the alternate allele at rs7869487;
(ii) the genotype of the sample is heterozygous for the reference allele at rs3810936 and is homozygous for the alternate allele at rs7869487;
(iii) the genotype of the sample is homozygous for the alternate allele at rs3810936 and is homozygous for the reference allele at rs7869487;
(iv) the genotype of the sample is homozygous for the alternate allele at rs3810936 and is heterozygous for the reference allele at rs7869487; or
(v) the genotype of the sample is homozygous for the alternate allele at rs3810936 and is homozygous for the alternate allele at rs7869487.

156. Use of an anti-TL1A antibody in the manufacture of a medicament for treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and
(b) an effective amount of the anti-TL1A antibody is to be administered to the patient, wherein the patient has been determined to be a haplotype B non-carrier, and
wherein the anti-TL1A antibody comprises a HCDR1 having the sequence shown in SEQ ID NO: 3, a HCDR2 having the sequence shown in SEQ ID NO: 4, a HCDR3 having the sequence shown in SEQ ID NO: 5, a LCDR1 having the sequence shown in SEQ ID NO: 6, a LCDR2 having the sequence shown in SEQ ID NO: 7, and a LCDR3 having the sequence shown in SEQ ID NO: 8.

157. Use of an anti-TL1A antibody in the manufacture of a medicament for treating IBD in a patient, wherein:
(a) a genotyping assay is to be performed to determine if the patient is a haplotype B carrier or a haplotype B non-carrier for TNFSF15, the assay comprising determining the presence of at least two SNPs selected from rs3810936, rs6478108, rs6478109, rs7848647, and rs7869487 in a biological sample from the patient; and
(b) the anti-TL1A antibody is to be administered to the patient, wherein the patient has been determined to be a haplotype B non-carrier, and
wherein the anti-TL1A antibody comprises a HCDR1 having the sequence shown in SEQ ID NO: 37, a HCDR2 having the sequence shown in SEQ ID NO: 39, a HCDR3 having the sequence shown in SEQ ID NO: 41, a LCDR1 having the sequence shown in SEQ ID NO: 46, a LCDR2 having the sequence shown in SEQ ID NO: 48, and a LCDR3 having the sequence shown in SEQ ID NO: 50.

158. Use of an anti-TL1A antibody in the manufacture of a medicament for treating an IBD in a patient, wherein an effective amount of the anti-TL1A antibody is to be administered to the subject in a dosing regimen comprising an induction dosing regimen and a subsequent maintenance dosing regimen;
wherein the induction dosing regimen comprises a plurality of individual induction doses and the maintenance dosing regimen comprising a plurality of individual maintenance doses;
wherein the anti-TL1A antibody is administered subcutaneously in the maintenance phase, and wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

159. Use of an anti-TL1A antibody in the manufacture of a medicament for treating an IBD in a patient, wherein an effective amount of the anti-TL1A antibody is to be administered to the subject in a dosing regimen comprising an induction dosing regimen and a subsequent maintenance dosing regimen, wherein:
(a) the induction dosing regimen comprises subcutaneous administration of four individual induction doses of the anti-TL1A antibody at a dose of about 50 mg, 150 mg, or 450 mg, wherein the individual induction doses are administered 4 weeks apart; and
(b) the maintenance dosing regimen comprises subcutaneous administration of a plurality of individual maintenance doses of the anti-TL1A antibody at a dose of about 50 mg, 150 mg, or 450 mg, wherein the individual maintenance doses are administered 4 weeks apart, and
wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.

160. Use of an anti-TL1A antibody in the manufacture of a medicament for treating an inflammatory bowel disease (IBD) in a patient, wherein an effective amount of the anti-TL1A antibody is to be administered to the subject in a dosing regimen comprising an induction dosing regimen and a subsequent maintenance dosing regimen, wherein:
(a) the induction dosing regimen comprises a plurality of individual induction doses, wherein the individual induction doses are separated from each other by at least 1 week, least 2 weeks, at least 3 weeks, or at least 4 weeks; and
(b) the maintenance dosing regimen comprises subcutaneous administration of a plurality of individual maintenance doses of the anti-TL1A antibody at a dose of about 450 mg, wherein the individual maintenance doses are administered 4 weeks apart, and
wherein the anti-TL1A antibody comprises:
a HCDR1 having the sequence shown in SEQ ID NO: 3,
a HCDR2 having the sequence shown in SEQ ID NO: 4,
a HCDR3 having the sequence shown in SEQ ID NO: 5,
a LCDR1 having the sequence shown in SEQ ID NO: 6,
a LCDR2 having the sequence shown in SEQ ID NO: 7, and
a LCDR3 having the sequence shown in SEQ ID NO: 8.


SEQUENCE LISTING (77 sequences)

SEQ ID NO: 1 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 2 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 3 - protein, 5 aa, synthetic construct
YYGIS

SEQ ID NO: 4 - protein, 17 aa, synthetic construct
WISTYNGNTHYARMLQG

SEQ ID NO: 5 - protein, 15 aa, synthetic construct
ENYYGSGAYRGGMDV

SEQ ID NO: 6 - protein, 11 aa, synthetic construct
RASQSVSSYLA

SEQ ID NO: 7 - protein, 7 aa, synthetic construct
DASNRAT

SEQ ID NO: 8 - protein, 9 aa, synthetic construct
QQRSNWPWT

SEQ ID NO: 9 - protein, 453 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGSYRGGMDVWGQGTTVTVSS

SEQ ID NO: 12 - protein, 124 aa, synthetic construct
QVQLVQSGAEVKKPGASVKVSCKASGYDFTYYGISWVRQAPGQGLEWMGWISTYNGNKHYARMLQGRVTMTTDTSTRTAYMELRSLRSDDTAVYYCARENYYGSGSYRGGMDVWGQGTTVTVSS

SEQ ID NO: 13 - protein, 124 aa, synthetic construct
QVQLVQSGAEVKKPGASVKVSCKASGYDFTYYGISWVRQAPGQGLEWMGWISTYNGGTHYARMLQGRVTMTTDTSTRTAYMELRSLRSDDTAVYYCARENYYGSGSYRGGMDVWGQGTTVTVSS

SEQ ID NO: 14 - protein, 124 aa, synthetic construct
QVQLVQSGAEVKKPGASVKVSCKASGYDFTYYGISWVRQAPGQGLEWMGWISTYNGVTHYARMLQGRVTMTTDTSTRTAYMELRSLRSDDTAVYYCARENYYGSGSYRGGMDVWGQGTTVTVSS

SEQ ID NO: 15 - protein, 124 aa, synthetic construct
QVQLVQSGAEVKKPGASVKVSCKASGYDFTYYGISWVRQAPGQGLEWMGWISTYNGGTHYARMLQGRVTMTTDTSTRTAYMELRSLRSDDTAVYYCARENYYGSGAYRGGMDAWGQGTTVTVSS

SEQ ID NO: 16 - protein, 124 aa, synthetic construct
QVQLVQSGAEVKKPGASVKVSCKASGYDFTYYGISWVRQAPGQGLEWMGWISTYNGVTHYARMLQGRVTMTTDTSTRTAYMELRSLRSDDTAVYYCARENYYGSGAYRGGMDAWGQGTTVTVSS

SEQ ID NO: 17 - protein, 124 aa, synthetic construct
QVQLVQSGAEVKKPGASVKVSCKASGYDFTYYGISWVRQAPGQGLEWMGWISTYNGKTHYARMHQGRVTMTTDTSTRTAYMELRSLRSDDTAVYYCARENYYGSGAYRGGMDAWGQGTTVTVSS

SEQ ID NO: 18 - protein, 124 aa, synthetic construct
QVQLVQSGAEVKKPGASLKVSCKASGYPFTNYGISWVRQAPGQGLEWMGWISTYNGNTHYAQKLQGRVTMTTDTSTTTAYMDLRSLRSDDTAVYYCARENYYGSGSYRGGMDVWGQGTTVTVSS

SEQ ID NO: 19 - protein, 124 aa, synthetic construct
QVQLVQSGAEVKKPGASVKVSCKASGYSFTTYGISWVRQAPGQGLEWMGWISTYNGNTHYAQKLQGRVTMTTDTSTRTAYMELRSLRSDDTAVYYCARENYYGSGSYRGGMDVWGQGTTVTVSS

SEQ ID NO: 20 - protein, 121 aa, synthetic construct
QVQLVQSGAEVKKPGASVKVSCKASGYTFTSYGINWVRQAPGQGLEWMGWISTYNGNTNSAQKLQGRVTMTTDTSTSTAYMELRSLRSDDTAVYYCARAHSSSWFDAFDIWGQGTMVTVSS

SEQ ID NO: 21 - protein, 126 aa, synthetic construct
QVQLVESGGGVVQPGRSLRLSCAASGFTFSSFAMHWVRQAPGKGLEWVALIPFDGSSNYYADSVKGRFTISRDNSKNTLYLQMNSLRAEDTAVYYCARDRNYYGSGSFSFDAFDIWGQGTLVTVSS

SEQ ID NO: 22 - protein, 126 aa, synthetic construct
QVQLVESGGGVVQPGRSLRLSCAASGFTFSNYALHWVRQAPGKGLEWVALISYDGSDKYYADSVKGRFAISRDNSKNTLYLQMNSLRAEDTAVYYCARDREYCTYSSCSYDAFDIWGQGTMVTVSS

SEQ ID NO: 23 - protein, 121 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGYSSAWFDAFDIWGQGTMVTVSS

SEQ ID NO: 24 - protein, 107 aa, synthetic construct [sequence omitted]

SEQ ID NO: 25 - protein, 107 aa, synthetic construct
DIQMTQSPSSLSASVGDRVTITCRASQGISNWLAWYQQKPEKAPKSLIYAASSLQSGVPSRFSGSGSGTDFTLTISSLQPEDFATYYCQQYNSYPYTFGQGTKLEIK

SEQ ID NO: 26 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDYATYYCQQYNSYPYTFGQGTKLEIK

SEQ ID NO: 27 - protein, 107 aa, synthetic construct [sequence omitted]

SEQ ID NO: 28 - protein, 121 aa, synthetic construct
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 29 - protein, 106 aa, synthetic construct
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 30 - protein, 449 aa, synthetic construct
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 216 aa, synthetic construct
QSVLTQPPSVSGAPGQRVTISCTSSSSDIGAGLGVHWYQQLPGTAPKLLIYGYYNRPSGVPDRFSGSKSGTSASLTITGLLPEDEGDYYCQSYDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 119 aa, synthetic construct
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 33 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 34 - protein, 454 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 30 aa, synthetic construct
QVQLVQSGAEVKKPGASVKVSCKASGFDIQ

SEQ ID NO: 37 - protein, 5 aa, synthetic construct
DTYMH

SEQ ID NO: 38 - protein, 14 aa, synthetic construct
WVKQRPGQGLEWMG

SEQ ID NO: 39 - protein, 17 aa, synthetic construct
RIDPASGHTKYDPKFQV

SEQ ID NO: 40 - protein, 32 aa, synthetic construct
RVTITRDTSTSTVYLELSSLRSEDTAVYYCAR

SEQ ID NO: 41 - protein, 7 aa, synthetic construct
SGGLPDV

SEQ ID NO: 42 - protein, 11 aa, synthetic construct
WGQGTTVTVSS

SEQ ID NO: 43 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 213 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 23 aa, synthetic construct
EIVLTQSPGTLSLSPGERATLSC

SEQ ID NO: 46 - protein, 10 aa, synthetic construct
RASSSVSYMY

SEQ ID NO: 47 - protein, 15 aa, synthetic construct
WYQQKPGQAPRPLIY

SEQ ID NO: 48 - protein, 7 aa, synthetic construct
ATSNLAS

SEQ ID NO: 49 - protein, 32 aa, synthetic construct
GIPDRFSGSGSGTDFTLTISRLEPEDFAVYYC

SEQ ID NO: 50 - protein, 9 aa, synthetic construct
QQWEGNPRT

SEQ ID NO: 51 - protein, 13 aa, synthetic construct
FGGGTKLEIKRTV

SEQ ID NO: 52 - protein, 104 aa, synthetic construct
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 54 - protein, 109 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTV

SEQ ID NO: 55 - protein, 10 aa, synthetic construct
GYTFTSYDIN

SEQ ID NO: 56 - protein, 10 aa, synthetic construct
WLNPNSGYTG

SEQ ID NO: 57 - protein, 10 aa, synthetic construct
EVPETAAFEY

SEQ ID NO: 58 - protein, 14 aa, synthetic construct
TSSSSDIGAGLGVH

SEQ ID NO: 59 - protein, 7 aa, synthetic construct
GYYNRPS

SEQ ID NO: 60 - protein, 10 aa, synthetic construct
QSWDGTLSAL

SEQ ID NO: 61 - protein, 119 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 62 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 63 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 216 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 10 aa, synthetic construct
GYDFTYYGIS

SEQ ID NO: 69 - protein, 30 aa, synthetic construct
QVQLVQSGAEVKKPGASVKVSCKASGYDFT

SEQ ID NO: 70 - protein, 14 aa, synthetic construct
WVRQAPGQGLEWMG

SEQ ID NO: 71 - protein, 32 aa, synthetic construct
RVTMTTDTSTRTAYMELRSLRSDDTAVYYCAR

SEQ ID NO: 72 - protein, 11 aa, synthetic construct
WGQGTTVTVSS

SEQ ID NO: 73 - protein, 23 aa, synthetic construct
EIVLTQSPATLSLSPGERATLSC

SEQ ID NO: 74 - protein, 15 aa, synthetic construct
WYQQKPGQAPRLLIY

SEQ ID NO: 75 - protein, 32 aa, synthetic construct
GIPARFSGSGSGTDFTLTISSLEPEDFAVYYC

SEQ ID NO: 76 - protein, 13 aa, synthetic construct
FGQGTKVEIKRTV

SEQ ID NO: 77 - protein, 25 aa, synthetic construct [sequence omitted]